(19) United States
(12) Patent Application Publication
(10) Pub. No.: US 2025/0057038 A1
(43) Pub. Date: Feb. 13, 2025

(54) COMPOUND FOR ORGANIC ELECTRIC ELEMENT, ORGANIC ELECTRIC ELEMENT USING THE SAME, AND AN ELECTRONIC DEVICE THEREOF

(71) Applicant: DUK SAN NEOLUX CO., LTD., Cheonan-si, Chungcheongnam-do (KR)

(72) Inventors: Moon Sung KANG, Cheonan-si, Chungcheongnam-do (KR); Jung Geun LEE, Cheonan-si, Chungcheongnam-do (KR); Sun Hee LEE, Cheonan-si, Chungcheongnam-do (KR); Soung Yun MUN, Cheonan-si, Chungcheongnam-do (KR); Yun Suk LEE, Cheonan-si, Chungcheongnam-do (KR); Hyun Jung BAEK, Cheonan-si, Chungcheongnam-do (KR)

(73) Assignee: DUK SAN NEOLUX CO., LTD., Cheonan-si, Chungcheongnam-do (KR)

(21) Appl. No.: 18/713,485

(22) PCT Filed: Nov. 4, 2022
(86) PCT No.: PCT/KR2022/017245

§ 371 Date: May 24, 2024

(30) Foreign Application Priority Data
Nov. 26, 2021 (KR) 10-2021-0166083

(51) Int. Cl. H10K 85/60; C07D 307/91; C07D 333/76; C07D 407/12; C07D 409/14; H10K 50/11; H10K 50/15

(52) U.S. Cl. CPC H10K 85/636; C07D 307/91; C07D 333/76; C07D 407/12; C07D 409/14; H10K 85/633; H10K 50/11; H10K 50/15; H10K 85/615; H10K 85/622; H10K 85/626; H10K 85/6572; H10K 85/6574; H10K 85/6576

(57) ABSTRACT

Provided are a compound represented by Formula 1, an organic electric element comprising a first electrode, a second electrode, and an organic material layer between the first electrode and the second electrode, and an electronic device thereof, where the compound represented by Formula 1 is included in the organic material layer and thereby the driving voltage of the organic electric element can be lowered, and the luminous efficiency and life time can be improved.

### CROSS-REFERENCE TO RELATED APPLICATION AND CLAIM OF PRIORITY

This patent application claims priority from and the benefit under 35 U.S.C. § 119 to § 121, and § 365 of Korean Patent Application No. 10-2021-0166083, filed on Nov. 26, 2021, and which is hereby incorporated by reference for all purposes as if fully set forth herein. Further, this application claims the benefit of priority in countries other than U.S., which is hereby incorporated by reference herein.

### BACKGROUND

### Technical Field

The present invention relates to an organic electric element comprising organic compound and electronic device thereof.

### Background Art

In general, an organic light emitting phenomenon refers to a phenomenon in which electric energy is converted into light energy of an organic material. An organic electric element utilizing the organic light emitting phenomenon usually has a structure including an anode, a cathode, and an organic material layer interposed therebetween.

In many cases, the organic material layer has a multi-layered structure having respectively different materials in order to improve efficiency and stability of an organic electric element, and for example, may comprise a hole injection layer, a hole transport layer, a light emitting layer, an electron transport layer, an electron injection layer, or the like.

The most important issues in organic electroluminescent elements are life and efficiency, and as the display becomes larger, these efficiency and life problems must be solved.

Efficiency, life span, driving voltage, and the like are correlated with each other. If efficiency is increased, then driving voltage is relatively lowered, and the crystallization of an organic material due to Joule heating generated during operation is reduced as driving voltage is lowered, as a result of which life span shows a tendency to increase.

However, efficiency cannot be maximized only by simply improving the organic material layer. This is because long life span and high efficiency can be simultaneously achieved when energy levels and T1 values among the respective layers included in the organic material layer, inherent material properties (mobility, interfacial properties, etc.) and the like are optimal combination.

Therefore, in order to fully demonstrate the excellent characteristics of organic electric element, it is necessary to develop materials that form the organic layer of the element, especially the light-emitting auxiliary layer.

### OBJECT, TECHNICAL SOLUTION AND EFFECTS OF THE INVENTION

The object of the present invention is to provide a compound capable of lowering the driving voltage of the device and improving the luminous efficiency and lifetime, an organic electric element comprising the same, and an electronic device thereof.

In an aspect of the present invention, the present invention provides compound represented by the following formula.

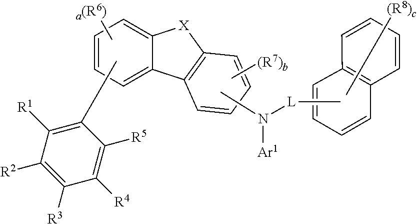

In another aspect of the present invention, the present invention provides an organic electric element employing compound represented by formula above and an electronic device thereof.

By using the compound according to embodiments of the present invention, the driving voltage of an organic electric element can be lowered, and the luminous efficiency and lifetime of the element can be improved.

### BRIEF DESCRIPTION OF THE DRAWINGS

FIGS. 1 to 3 illustrate an example of organic electroluminescent element according to the present invention.

### DESCRIPTION OF REFERENCE NUMERALS

100, 200, 300: organic electric element
110: first electrode
120: hole injection layer
130: hole transport layer
140: light-emitting layer
150: electron transport layer
160: electron injection layer
170: second electrode
180: layer for improving light efficiency
210: buffer layer
220: light-emitting auxiliary layer
320: first hole injection layer
330: first hole transport layer
340: first light-emitting layer
350: first electron transport layer
360: first charge generation layer
361: second charge generation layer
420: second hole injection layer
430: second hole transport layer
440: second light-emitting layer
450: second electron transport layer
CGL: charge generation layer
ST1: first stack
ST2: second stack

### DETAILED DESCRIPTION

Unless otherwise stated, the term “aryl group” or “arylene group” as used herein has, but not limited to, 6 to 60 carbon atoms. The aryl group or arylene group in the present invention may comprise a monocyclic ring, ring assemblies, a fused polycyclic system, spiro compound and the like.

As used herein, the term “fluorenyl group” refers to a substituted or unsubstituted fluorenyl group, and “fluorenylene group” refers to a substituted or unsubstituted fluorenylene group. The fluorenyl group or fluorenylene group used in the present invention comprises a spiro compound formed by combining R and R′ with each other in the following structure, and also comprises compound formed by linking adjacent R″s to each other. “Substituted fluorenyl group”, “substituted fluorenylene group” means that at least one of R, R′, R″ in the following structure is a substituent other than hydrogen, and R″ may be 1 to 8 in the following formula. In this specification, a fluorenyl group, a fluorenylene group, and the like may be referred to as a fluorene group or fluorene regardless of the valence.

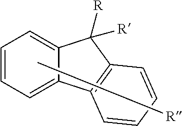

The term “spiro compound” as used herein has a spiro union which means union having one atom as the only common member of two rings. Here, the atom shared between the two rings is called as a ‘spiro atom’ and the compounds are called ‘monospiro-’, ‘dispiro-’ or ‘trispiro-’ depending on the number of spiro atoms in one compound.

The term “heterocyclic group” used in the specification comprises a non-aromatic ring as well as an aromatic ring like “heteroaryl group” or “heteroarylene group”. Unless otherwise stated, the term “heterocyclic group” means, but not limited to, a ring containing one or more heteroatoms and having 2 to 60 carbon atoms. Unless otherwise stated, the term “heteroatom” as used herein represents, for example, N, O, S, P or Si and may comprise a heteroatom group such as SO₂, P═O etc. instead of carbon forming a ring such as the following compound. In the specification, “heterocyclic group” comprises a monocyclic, ring assemblies, fused polycyclic system, a spiro-compound and the like.

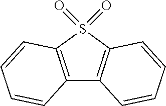 [SMILES: O=S1(=O)c2ccccc2-c2ccccc21]

The term “aliphatic ring group” as used herein refers to a cyclic hydrocarbon except for aromatic hydrocarbons, and comprises a monocyclic ring, ring assemblies, a fused polycyclic system, a spiro-compound and the like, and unless otherwise specified, it means a ring of 3 to 60 carbon atoms, but not limited thereto. For example, a fused ring of benzene being an aromatic ring and cyclohexane being a non-aromatic ring corresponds to aliphatic ring group.

In this specification, a ‘group name’ corresponding to an aryl group, an arylene group, a heterocyclic group, and the like exemplified for each symbol and its substituent may be written in the name of functional group reflecting the valence, and may also be described as the name of a parent compound. For example, in the case of phenanthrene which is a kind of aryl group, it may be described by distinguishing valence such as ‘phenanthryl (group)’ when it is ‘monovalent group’, and as ‘phenanthrylene (group)’ when it is ‘divalent group’, and it may also be described as a parent compound name, ‘phenanthrene’, regardless of its valence. Similarly, in the case of pyrimidine, it may be described as ‘pyrimidine’ regardless of its valence, and it may also be described as the name of corresponding functional group such as pyrimidinyl (group) when it is ‘monovalent group’, and as ‘pyrimidylene (group)’ when it is ‘divalent group’.

In addition, in the present specification, the numbers and alphabets indicating a position may be omitted when describing a compound name or a substituent name, For example, pyrido[4,3-d]pyrimidine, benzopuro[2,3-d]pyrimidine and 9,9-dimethyl-9H-fluorene can be described as pyridopyrimidine, benzofurropyrimidine and dimethylfluorene, respectively. Therefore, both benzo[g]quinoxaline and benzo[f]quinoxaline can be described as benzoquinoxaline.

In addition, unless otherwise expressed, where any formula of the present invention is represented by the following formula, the substituent according to the index may be defined as follows.

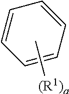

In the above formula, where a is an integer of zero, the substituent R¹ is absent, that is, hydrogen atoms are bonded to all the carbon constituting the benzene ring.

Here, chemical formulas or compounds may be written without indicating the hydrogen bonded to carbon. In addition, one substituent R¹ is bonded to any carbon of the carbons forming the benzene ring when “a” is an integer of 1. Similarly, where “a” is an integer of 2 or 3, substituents R¹s may be bonded to the carbon of the benzene ring, for example, as follows. Also, where “a” is an integer of 4 to 6, substituents R¹s are bonded to the carbon of the benzene ring in a similar manner. Further, where “a” is an integer of 2 or more, R¹s may be the same or different from each other.

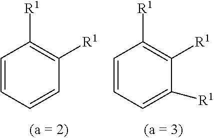

In addition, unless otherwise specified in the specification, the term ‘ring’ refers to an aryl ring, heteroaryl ring, fluorene ring, aliphatic ring, etc., and a number-membered (atom) ring may refer to the shape of a ring. For example, naphthalene corresponds to a two-fused (condensed) ring, anthracene to a three-fused (condensed) ring, thiophene or furan corresponds to a five-membered ring, and benzene or pyridine corresponds to a six-membered ring.

In addition, unless otherwise specified in the present specification, when adjacent groups are linked to each other to form a ring, the ring may be selected from the group consisting of a C₆-C₆₀ aromatic ring group, a fluorenyl group, a C₂-C₆₀ heterocyclic group containing at least one heteroatom selected from the group consisting of O, N, S, Si and P, and a C₃-C₆₀ aliphatic ring. Here, the aromatic ring group may include an aryl ring, and the heterocyclic group may include a heteroaryl ring.

Unless otherwise stated, the term “between adjacent groups”, for example, in case of the following Formulas, comprises not only “between R₁ and R₂”, “between R₂ and R₃”, “between R₃ and R₄”, “between R₅ and R₆”, but also “between R₇ and R₈” sharing one carbon, and may comprise “between substituents” attached to atom (carbon or nitrogen) consisting different ring, such as “between R₁ and R₇”, “between R₁ and R₈”, or “between R₄ and R₅” and the like. That is, where there are substituents bonded to adjacent elements constituting the same ring, the substituents may be correspond “adjacent groups,” and even if there are no adjacent substituents on the same ring, substituents attached to the adjacent ring may correspond to “adjacent groups.” In the following Formula, when the substituents bonded to the same carbon, such as R₇ and R₈, are linked to each other to form a ring, a compound containing a spiro-moiety may be formed.

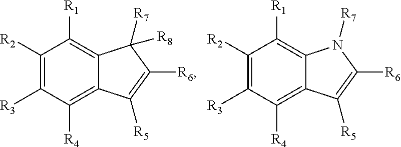

In addition, in the present specification, the expression ‘adjacent groups may be linked to each other to form a ring’ is used in the same sense as ‘adjacent groups are linked selectively to each other to form a ring’, and a case where at least one pair of adjacent groups may be bonded to each other to form a ring.

In addition, unless otherwise specified in the present specification, an aryl group, an arylene group, a fluorenyl group, a fluorenylene group, a heterocyclic group, an aliphatic ring group, an alkyl group, an alkenyl group, an alkynyl group, an alkoxyl group, an aryloxyl group, and a ring formed by adjacent groups may be each optionally substituted with one or more substituents selected from the group consisting of deuterium, halogen, an amino group unsubstituted or substituted with a C₁-C₂₀ alkyl group or a C₆-C₂₀ aryl group, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group.

Hereinafter, referring to FIGS. 1 to 3, a lamination structure of an organic electric element including the compound of the present invention will be described.

In designation of reference numerals to components in respective drawings, it should be noted that the same elements will be designated by the same reference numerals although they are shown in different drawings. In addition, in the following description of the present invention, a detailed description of known functions and configurations incorporated herein will be omitted when it may make the subject matter of the present invention rather unclear.

Terms, such as first, second, A, B, (a), (b) or the like may be used herein when describing components of the present invention. Each of these terminologies is not used for defining an essence, order or sequence of a corresponding component but used merely to distinguish the corresponding component from other component(s). It will be understood that the expression ‘one component is “connected,” “coupled” or “joined” to another component’ comprises the case where a third component may be “connected,” “coupled” or “joined” between a first component and a second component as well as the case where the first component may be directly connected, coupled or joined to the second component.

In addition, it will be understood that when an element such as a layer, film, region, or substrate is referred to as being “on” or “over” another element, it can be directly on the other element, or intervening elements may also be present. In contrast, when an element is referred to as being “directly on” another element, there are no intervening elements present.

FIGS. 1 to 3 show an example of an organic electric element according to an embodiment of the present invention, respectively.

Referring to the FIG. 1, an organic electric element 100 according to an embodiment of the present invention includes a first electrode 110 formed on a substrate (not shown), a second electrode 170, and an organic material layer between the first electrode 110 and the second electrode 170.

The first electrode 110 may be an anode (positive electrode), and the second electrode 170 may be a cathode (negative electrode). In the case of an inverted organic electric element, the first electrode may be a cathode, and the second electrode may be an anode.

The organic material layer may be comprised a hole injection layer 120, a hole transport layer 130, a light emitting layer 140, an electron transport layer 150, and an electron injection layer 160. Specifically, a hole injection layer 120, a hole transport layer 130, a light-emitting layer 140, an electron transport layer 150, and an electron injection layer 160 may be formed on the first electrode 110 in sequence.

Preferably, a layer for improving the luminous efficiency 180 may be formed one side of sides of the first electrode 110 and the second electrode 170, wherein one side is not facing the organic material layer, as a result the luminous efficiency of an organic electric element can be improved.

For example, the light efficiency improving layer 180 may be formed on the second electrode 170, as a result, in the case of a top emission organic light emitting element, the optical energy loss due to Surface Plasmon Polaritons (SPPs) at the second electrode 170 may be reduced and in the case of a bottom emission organic light emitting element, the light efficiency improving layer 180 may serve as a buffer for the second electrode 170.

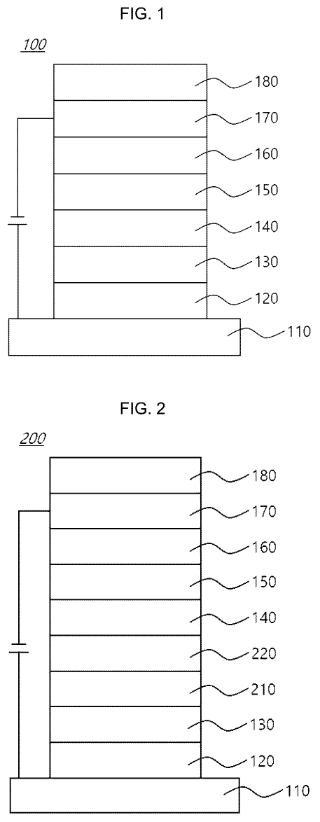

A buffer layer 210 or a light-emitting auxiliary layer 220 may be further formed between the hole transport layer 130 and the light emitting layer 140, which will be described with reference to FIG. 2.

Referring to FIG. 2, the organic electric element 200 according to another embodiment of the present invention may comprise a hole injection layer 120, a hole transport layer 130, a buffer layer 210, a light-emitting auxiliary layer 220, a light emitting layer 140, the electron transport layer 150, the electron injection layer 160, and a second electrode 170 formed on a first electrode 110 in sequence, and a layer for improving light efficiency 180 may be formed on the second electrode.

Although not shown in FIG. 2, an electron transport auxiliary layer may be further formed between the light emitting layer 140 and the electron transport layer 150.

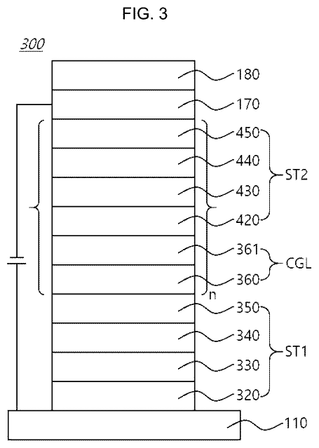

In addition, according to another embodiment of the present invention, the organic material layer may be a form consisting of multiple stacks, wherein the stacks comprise a hole transport layer, a light emitting layer, and an electron transport layer, respectively. This will be described with reference to FIG. 3.

Referring to FIG. 3, two or more sets of stacks of the organic material layers ST1 and ST2 may be formed between the first electrode 110 and the second electrode 170 in the organic electric element 300 according to another embodiment of the present invention, wherein the organic material layers are consisted of multiple layers, respectively, and the charge generation layer CGL may be formed between the stacks of the organic material layer.

Specifically, the organic electric element according to the embodiment of the present invention may comprise a first electrode 110, a first stack ST1, a charge generation layer CGL, a second stack ST2, and a second electrode 170 and a layer for improving light efficiency 180.

The first stack ST1 is an organic layer formed on the first electrode 110, and the first stack ST1 may comprise the first hole injection layer 320, the first hole transport layer 330, the first light emitting layer 340 and the first electron transport layer 350 and the second stack ST2 may comprise a second hole injection layer 420, a second hole transport layer 430, a second light emitting layer 440 and a second electron transport layer 450. As such, the first stack and the second stack may be the organic layers having the same or different stacked structures.

The charge generation layer CGL may be formed between the first stack ST1 and the second stack ST2. The charge generation layer CGL may comprise a first charge generation layer 360 and a second charge generation layer 361. The charge generating layer CGL is formed between the first light emitting layer 340 and the second light emitting layer 440 to increase the current efficiency generated in each light emitting layer and to smoothly distribute charges.

The first light emitting layer 340 may comprise a light emitting material comprising a blue host doped with a blue fluorescent dopant and the second light emitting layer 440 may comprise a light emitting material comprising a green host doped with a greenish yellow dopant and a red dopant together, but the material of the first light emitting layer 340 and the second light emitting layer 440 according to an embodiment of the present invention is not limited thereto.

In FIG. 3, n may be an integer of 1 to 5 and the charge generation layer CGL and the third stack may be further stacked on the second stack ST2 when n is 2.

When a plurality of light-emitting layers are formed in a multi-layer stack structure as shown in FIG. 3, it is possible to manufacture an organic electroluminescent element that emits not only white light but also various colors, wherein the white light is emitted by the mixing effect of light emitted from each light emitting layer.

Compound(s) represented by Formula 1 of the present invention may be included in an organic layer. For example, the compounds represented by Formulas 1 of the present invention can be used as a material for a hole injection layer 120, 320, 420, a hole transport layer 130, 330, 430, a buffer layer 210, a light-emitting auxiliary layer 220, and an electron transport layer 150, 350, 450, an electron injection layer 160, a light-emitting layer 140, 340, 440, or a layer for improving light efficiency 180, preferably, a hole transport layer 130, 330, 430, a light-emitting auxiliary layer 220, or/and a layer for improving light efficiency 180, more preferably, a light-emitting auxiliary layer 220.

Even if the cores of compounds are identical or similar to each other, the band gap, the electrical characteristics, the interface characteristics, and the like may be different depending on which substituent is bonded at which position. Therefore, it is necessary to study the selection of the core and the combination with sub-substituent bonded to the core. In particular, long life span and high efficiency can be simultaneously achieved when the optimal combination of energy levels and T₁ values, inherent material properties (mobility, interfacial properties, etc.), and the like among the respective layers of an organic material layer is achieved.

Therefore, the energy level and T₁ value between the respective layers of the organic material layer, inherent material properties (mobility, interfacial properties, etc.) and the like can be optimized by using the compound represented by Formula 1 as a material for a light-emitting auxiliary layer 220.

The organic electric element according to an embodiment of the present invention may be manufactured using various deposition methods. The organic electric element according to an embodiment of the present invention may be manufactured using a PVD (physical vapor deposition) method or CVD (chemical vapor deposition) method.

For example, the organic electric element may be manufactured by depositing a metal, a conductive metal oxide, or a mixture thereof on the substrate to form the anode 110, forming the organic material layer comprising the hole injection layer 120, the hole transport layer 130, the light emitting layer 140, the electron transport layer 150, and the electron injection layer 160 thereon, and then depositing a material, which can be used as the cathode 170, thereon.

In addition, a light-emitting auxiliary layer 220 may be formed between a hole transport layer 130 and a light emitting layer 140, and an electron transport auxiliary layer (not shown) may be further formed between a light emitting layer 140 and an electron transport layer 150 and, as described above, a stack structure may be formed.

In addition, the organic material layer may be manufactured in such a manner that the fewer layers are formed using various polymer materials by a soluble process or solvent process, for example, spin coating, nozzle printing, inkjet printing, slot coating, dip coating, roll-to-roll, doctor blading, screen printing, or thermal transfer, instead of deposition. Since the organic material layer according to the present invention may be formed in various ways, the scope of protection of the present invention is not limited by a method of forming the organic material layer.

The organic electric element according to an embodiment of the present invention may be of a top emission type, a bottom emission type, or a dual emission type depending on the material used.

In addition, the organic electric element according to an embodiment of the present invention may be selected from the group consisting of an organic electroluminescent element, an organic solar cell, an organic photo conductor, an organic transistor, an element for monochromatic illumination and an element for a quantum dot display.

Another embodiment of the present invention provides an electronic device including a display device which includes the above-described organic electric element, and a control unit for controlling the display device. Here, the electronic device may be a wired/wireless communication terminal which is currently used or will be used in the future and covers all kinds of electronic devices including a mobile communication terminal such as a cellular phone, a navigation unit, a game player, various kinds of TVs, and various kinds of computers.

Hereinafter, compounds according to one aspect of the present invention will be described.

Compound according to an aspect of the present invention is represented by Formula 1 below.

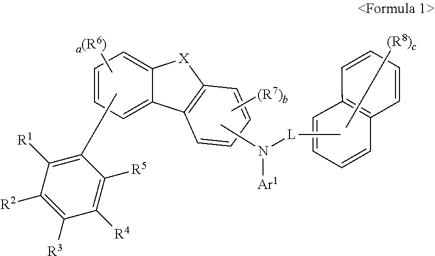

In Formula 1, each of symbols may be defined as follows.

X is O or S.

R¹ to R⁵ are each independently selected from the group consisting of hydrogen, deuterium, halogen, a cyano group, a nitro group, a C₆-C₆₀ aryl group, a fluorenyl group, a C₂-C₆₀ heterocyclic group containing at least one heteroatom of O, N, S, Si and P, a C₃-C₆₀ aliphatic ring group, a C₁-C₂₀ alkyl group, a C₂-C₂₀ alkenyl group, a C₂-C₂₀ alkynyl group, a C₁-C₂₀ alkoxyl group, and a C₆-C₂₀ aryloxy group, and adjacent groups may be bonded to each other to form a ring of 6-membered or more, and the case where R¹ to R⁵ are all hydrogen is excluded.

In one embodiment, at least one of R¹ to R⁵ may be selected from the group consisting of an aryl group, an alkyl group, and a cycloalkyl group.

A ring, formed by at least one pair of adjacent R¹ and R², R² and R³, R³ and R⁴, and R⁴ and R⁵, may be selected from the group consisting of a C₆-C₆₀ aromatic ring group, a fluorenyl group, a C₂-C₆₀ heterocyclic group containing at least one heteroatom of O, N, S, Si and P, and a C₆-C₆₀ aliphatic ring, and the ring is a ring of a six-membered or more. For examples, when an aromatic ring is formed by adjacent groups, the aromatic ring may be, for example, a monocyclic ring of a six-membered ring, such as benzene, naphthalene, anthracene, phenanthrene, pyrene, etc. or a polycyclic aryl ring in which six-membered rings are condensed with each other.

R⁶ to R⁸ are each independently selected from the group consisting of hydrogen, deuterium, halogen, a cyano group, a nitro group, a C₆-C₆₀ aryl group, a fluorenyl group, a C₂-C₆₀ heterocyclic group containing at least one heteroatom of O, N, S, Si and P, a C₃-C₆₀ aliphatic ring group, a C₁-C₂₀ alkyl group, a C₂-C₂₀ alkenyl group, a C₂-C₂₀ alkynyl group, a C₁-C₂₀ alkoxyl group, and a C₆-C₂₀ aryloxy group, and adjacent groups may be bonded to each other to form a ring.

a and b are each an integer of 0 to 3, c is an integer of 0 to 7, and when each of these is an integer of 2 or more, each of R⁶s, each of R⁷s, each of R⁸s are the same as or different from each other.

A ring, formed by at least one pair of adjacent R⁶s, R⁷s, and R⁸s, may be selected from the group consisting of a C₆-C₆₀ aromatic ring group, a fluorenyl group, a C₂-C₆₀ heterocyclic group containing at least one heteroatom of O, N, S, Si and P, and a C₆-C₆₀ aliphatic ring.

When an aromatic ring is formed by adjacent groups, the aromatic ring may be, for example, a C₆-C₂₀, a C₆-C₁₈, a C₆-C₁₆, a C₆-C₁₄, a C₆-C₁₃, a C₆-C₁₂, a C₆-C₁₀, a C₆, a C₁₀, a C₁₂, a C₁₄, a C₁₅, a C₁₆, or a C₁₈ aromatic ring, specifically, an aryl ring such as benzene, naphthalene, anthracene, phenanthrene, pyrene, etc.

Ar¹ is selected from the group consisting of a C₆-C₆₀ aryl group, a fluorenyl group, a C₂-C₆₀ heterocyclic group containing at least one heteroatom of O, N, S, Si and P, and a C₃-C₆₀ aliphatic ring group.

L is selected from the group consisting of a non-fused C₆-C₆₀ arylene group, a fluorenylene group, a C₃-C₆₀ aliphatic ring group, and a C₂-C₆₀ heterocyclic group containing at least one heteroatom of O, N, S, Si and P. Preferably, the L may be a non-fused C₆-C₆₀ arylene group, for example, a non-fused aryl ring made of one or more uncondensed monocycles, such as phenylene, biphenyl, terphenyl, etc.

When at least one of R¹ to R⁸, Ar¹ is an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, or a C₁₈ aryl group, specifically, phenyl, biphenyl, naphthyl, terphenyl, phenanthrene, triphenylene, or the like.

When L is a non-fused arylene group, the non-fused arylene group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈ arylene group which is a non-fused arylene group, specifically, phenylene, biphenyl, terphenyl, or the like.

When at least one of R¹ to R⁸, Ar¹, L is a heterocyclic group, the heterocyclic group may be, for example, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, or a C₂₉ heterocyclic group, specifically, pyridine, pyrimidine, pyrazine, pyridazine, triazine, furan, pyrrole, silole, indene, indole, phenyl-indole, benzoindole, phenyl-benzoindole, pyrazinoindol, quinoline, isoquinoline, benzoquinoline, pyridoquinoline, quinazoline, benzoquinazoline, dibenzoquinazoline, phenanthroquinazoline, quinoxaline, benzoquinoxaline, dibenzoquinoxaline, benzofuran, naphthobenzofuran, dibenzofuran, dinaphthofuran, thiophene, benzothiophene, dibenzothiophene, naphthobenzothiophene, dinaphthothiophene, carbazole, phenyl-carbazole, benzocarbazole, phenyl-benzocarbazole, naphthyl-benzocarbazole, dibenzocarbazole, indolocarbazole, benzofuropyridine, benzothienopyridine, benzofuropyridine, benzothienopyrimidine, benzofuropyrimidine, benzothienopyrazine, benzofuropyrazine, benzoimidazole, benzothiazole, benzooxazole, benzosiloe, phenanthroline, dihydro-phenylphenazine, 10-phenyl-10H-phenoxazine, phenoxazine, phenothiazine, dibenzodioxin, benzodibenzodioxin, thianthrene, 9,9-dimethyl-9H-xanthene, 9,9-dimethyl-9H-thioxanthene, dihydrodimethylphenylacridine, spiro[fluorene-9,9′-xanthene] and the like.

When at least one of R¹ to R⁸, Ar¹ is a fluorenyl group or L is a fluorenylene group, the fluorenyl group or the fluorenylene group may be, for example, 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

When at least one of R¹ to R⁸, Ar¹ is an aliphatic ring group, the aliphatic ring group, may be, for example, a C₃-C₂₀, a C₃-C₁₉, a C₃-C₁₈, a C₃-C₁₇, a C₃-C₁₆, a C₃-C₁₅, a C₃-C₁₄, a C₃-C₁₃, a C₃-C₁₂, a C₃-C₁₁, a C₃-C₁₀, a C₃-C₈, a C₃-C₆, a C₆, a C₁₀, a C₁₁, a C₁₂, a C₁₃, a C₁₄, a C₁₅, a C₁₆, a C₁₇ or a C₁₈ aliphatic ring group.

When at least one of R¹ to R⁸ is an alkyl group, the alkyl group may be, for example, a C₁-C₂₀, a C₁-C₁₀, a C₁-C₄, a C₁, a C₂, a C₃ and a C₄ alkyl group, for example, methyl, ethyl, t-butyl, etc.

The aryl group, the arylene group, the fluorenyl group, the fluorenylene group, the heterocyclic group, the aliphatic ring group, the alkyl group, the alkenyl group, the alkynyl group, the alkoxyl group, the aryloxyl group, and the ring formed by adjacent groups may be each optionally substituted with one or more substituents selected from the group consisting of deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group.

When at least one of the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, the ring formed by adjacent groups is substituted with an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, a C₂₉, or a C₃₀ aryl group.

When at least one of the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, the ring formed by adjacent groups is substituted with a heterocyclic group, the heterocyclic group may be, for example, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, a C₂₉, or a C₃₀ heterocyclic group.

When at least one of the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, the ring formed by adjacent groups is substituted with a fluorenyl group, the fluorenyl group may be 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

When at least one of the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, the ring formed by adjacent groups is substituted with an alkyl group, the alkyl group may be, for example, a C₁-C₂₀, a C₁-C₁₀, a C₁-C₄, a C₁, a C₂, a C₃, or a C₄ alkyl group.

When at least one of the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, the ring formed by adjacent groups is substituted with an aliphatic ring group, the aliphatic ring group may be, for example, a C₃-C₂₀, a C₃-C₁₉, a C₃-C₁₈, a C₃-C₁₇, a C₃-C₁₆, a C₃-C₁₅, a C₃-C₁₄, a C₃-C₁₃, a C₃-C₁₂, a C₃-C₁₁, a C₃-C₁₀, a C₃-C₈, a C₃-C₆, a C₆, a C₁₀, a C₁₁, a C₁₂, a C₁₃, a C₁₄, a C₁₅, a C₁₆, a C₁₇ or a C₁₈ aliphatic ring group.

Formula 1 may be represented by one of the following Formula 1-1 to Formula 1-44.

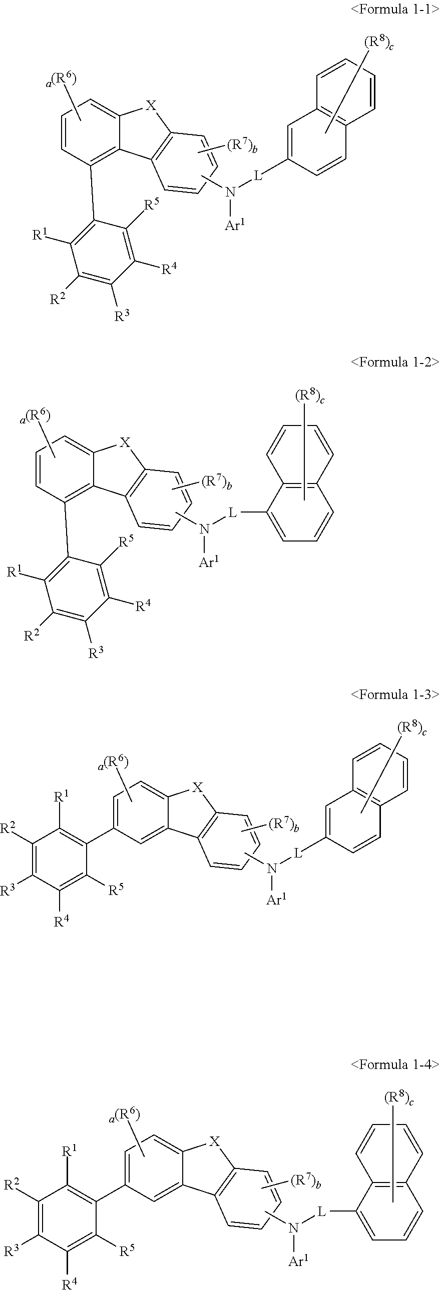

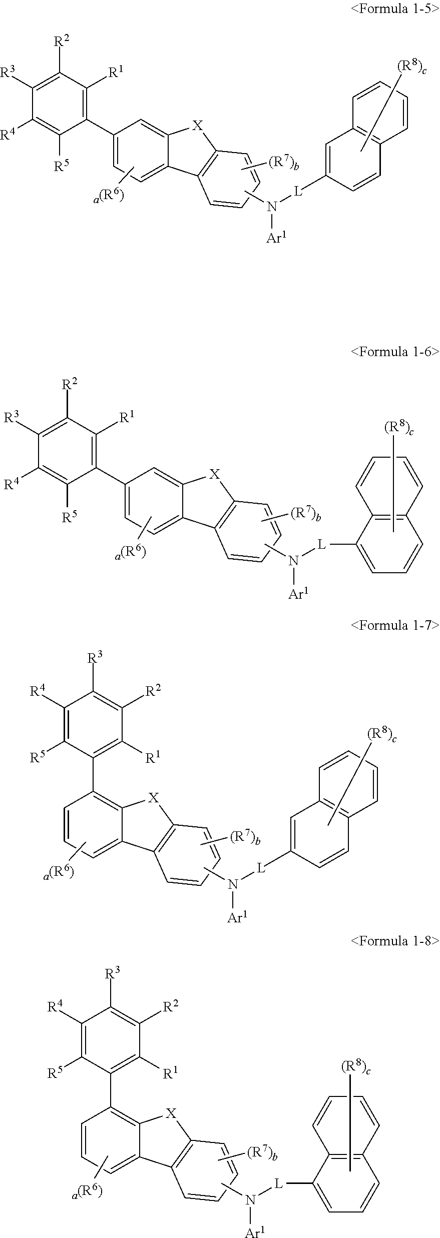

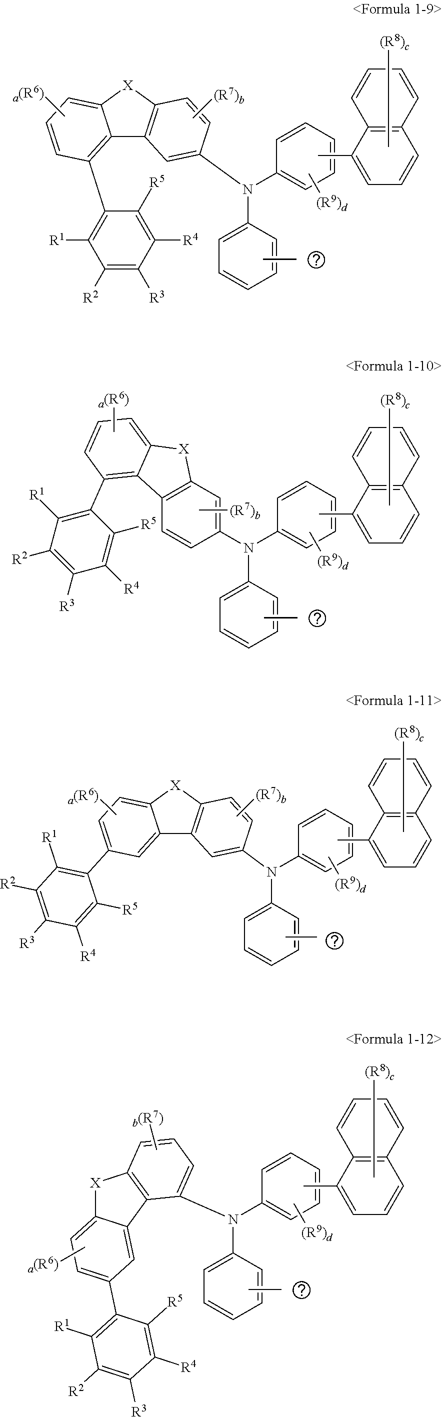

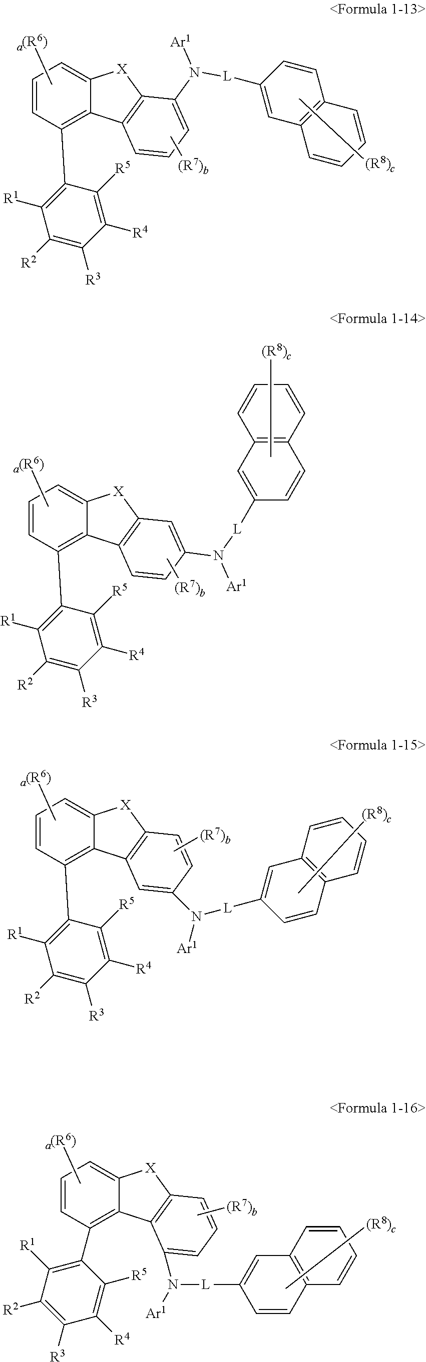

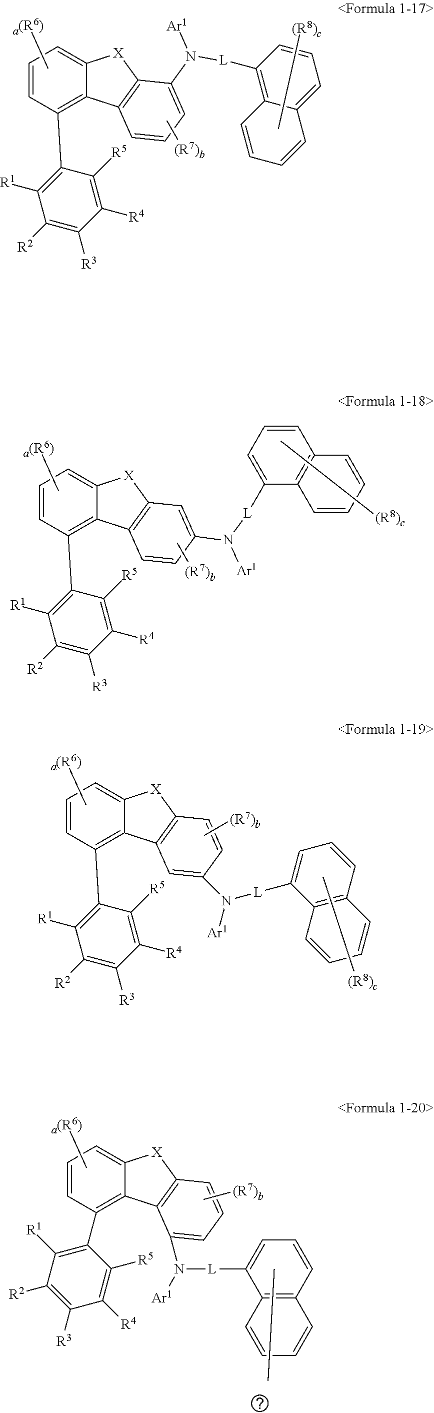

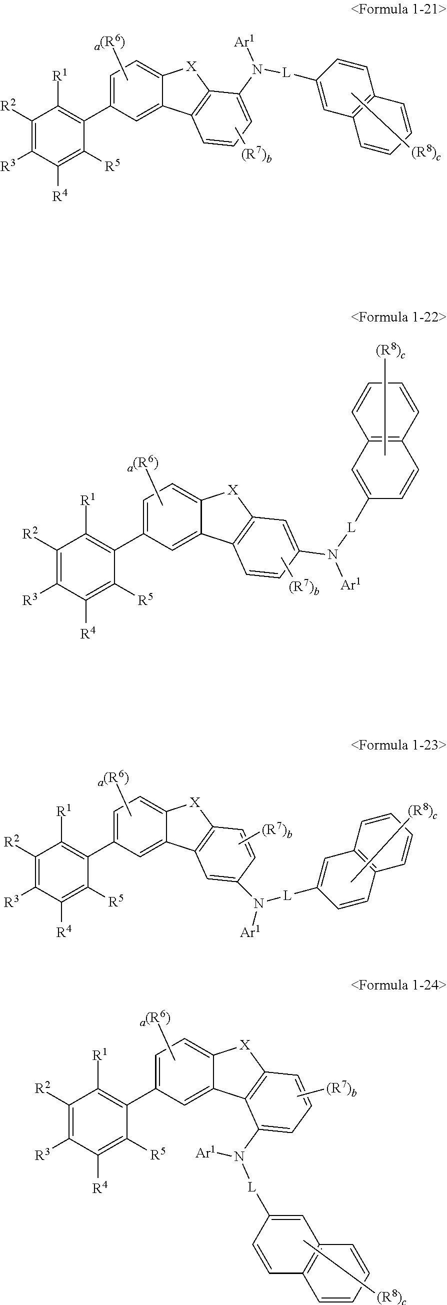

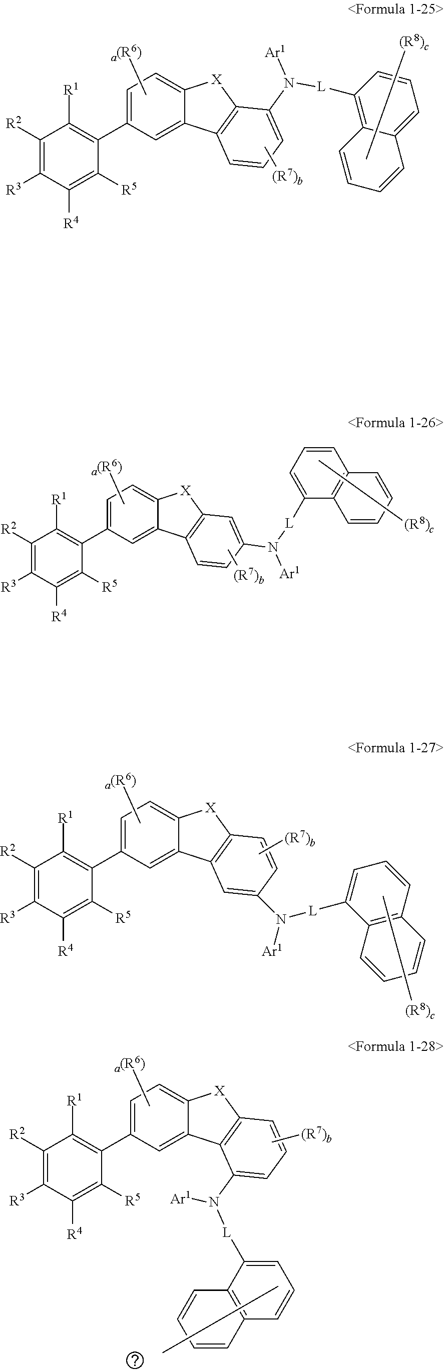

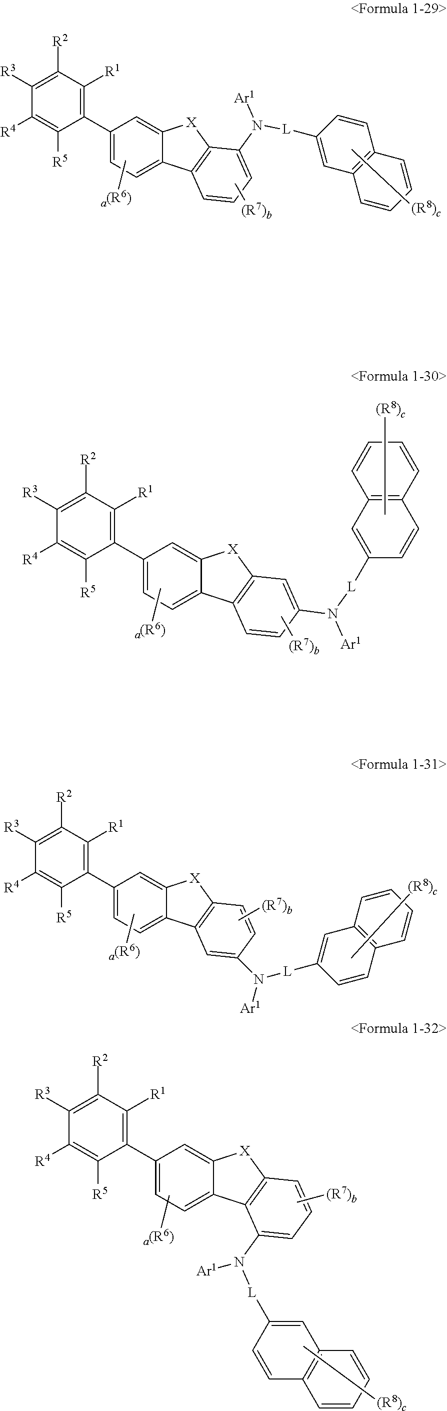

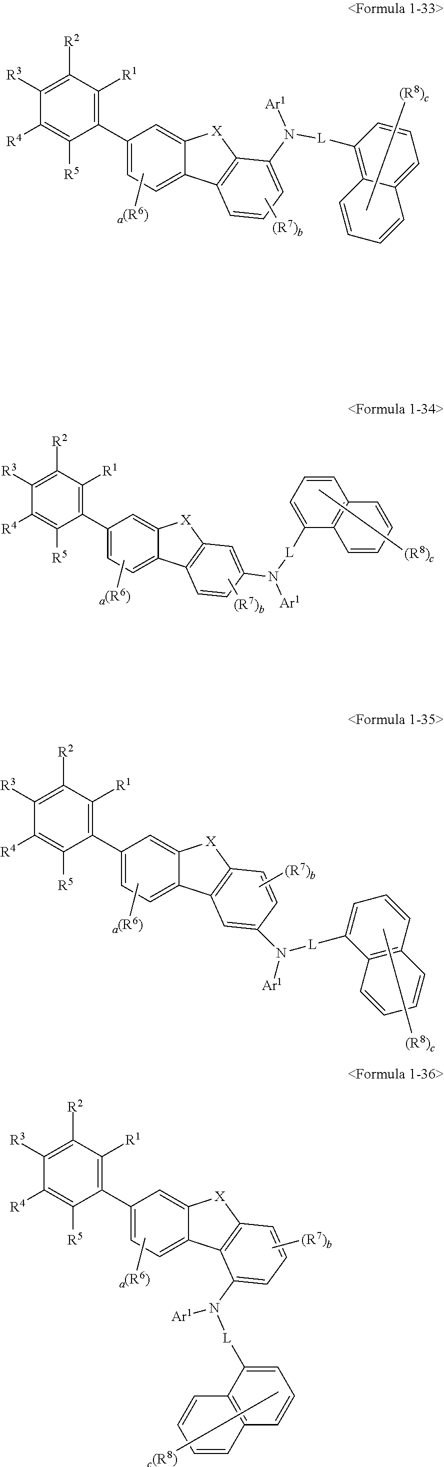

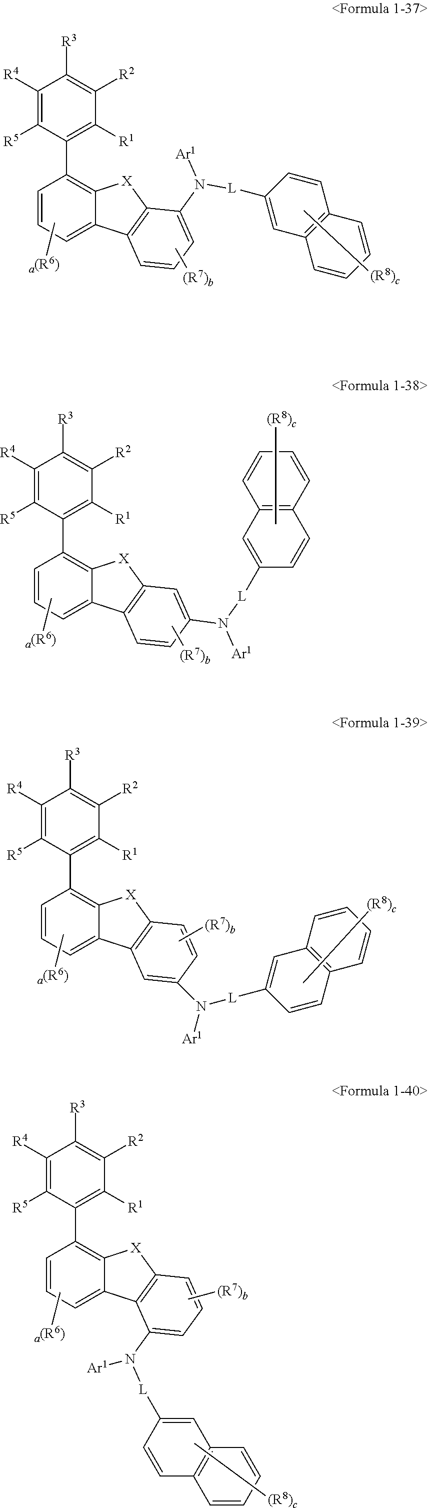

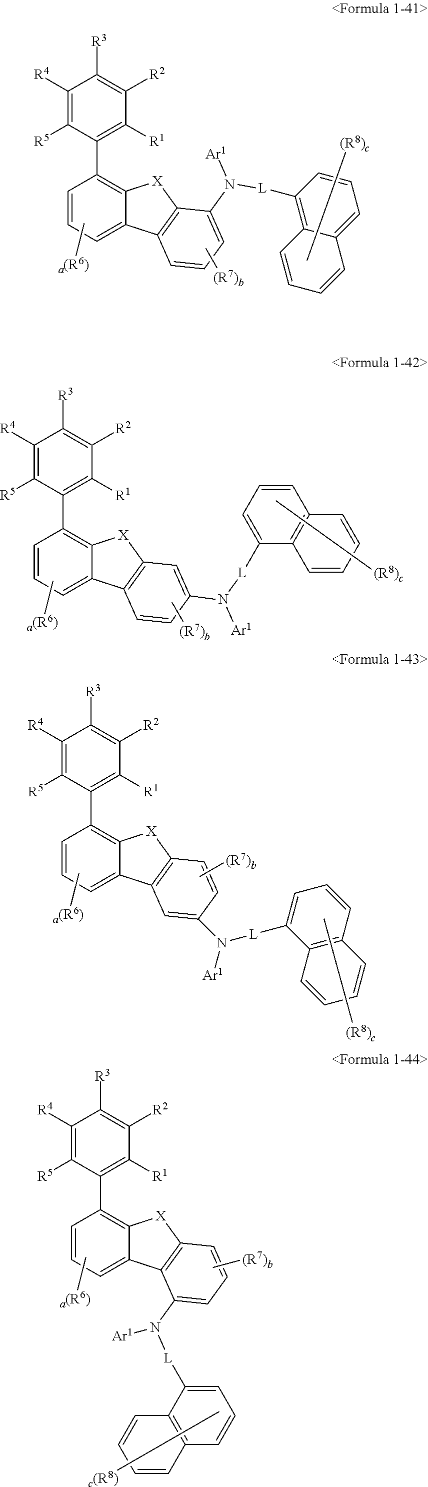

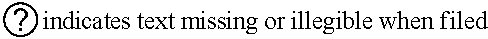

In Formula 1-1 to Formula 1-44, X, Ar¹, L, R¹ to R⁸, a to c are the same as defined for Formula 1.

R⁹ and R^{a} are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and adjacent R^{a}s may be bonded to each other to form a ring.

A ring formed by adjacent R^{a}s may be selected from the group consisting of a C₆-C₆₀ aromatic ring group, a fluorenyl group, a C₂-C₆₀ heterocyclic group containing at least one heteroatom of O, N, S, Si and P, and a C₆-C₆₀ aliphatic ring.

When an aromatic ring is formed by adjacent R^{a}s, the aromatic ring may be, for example, a C₆-C₂₀, a C₆-C₁₈, a C₆-C₁₆, a C₆-C₁₄, a C₆-C₁₃, a C₆-C₁₂, a C₆-C₁₀, a C₆, a C₁₀, a C₁₂, a C₁₄, a C₁₅, a C₁₆, or a C₁₈ aromatic ring, specifically, an aryl ring such as benzene, naphthalene, anthracene, phenanthrene, pyrene, etc.

d is an integer of 0 to 4, e is an integer of 0 to 5, and when each of these is an integer of 2 or more, each of R⁹s, each of R^{a}s are the same as or different from each other.

The above Formula, L is one of the following Formula L-1 to Formula L-3.

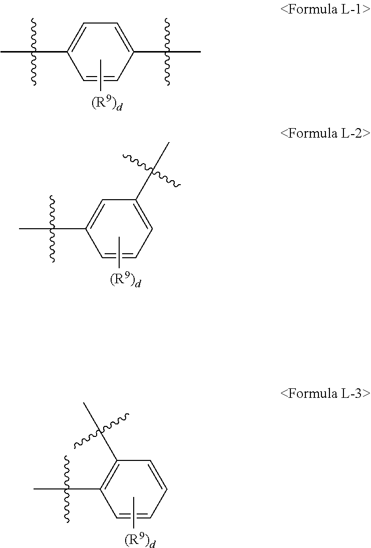

In Formula L-1 to Formula L-3, R⁹ is selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and d is an integer of 0 to 4, when d is an integer of 2 or more, each of R⁹s are the same as or different from each other,

At least one of R¹ to R⁵, Ar¹ is selected from the group consisting of Formula C-1 to Formula C-9.

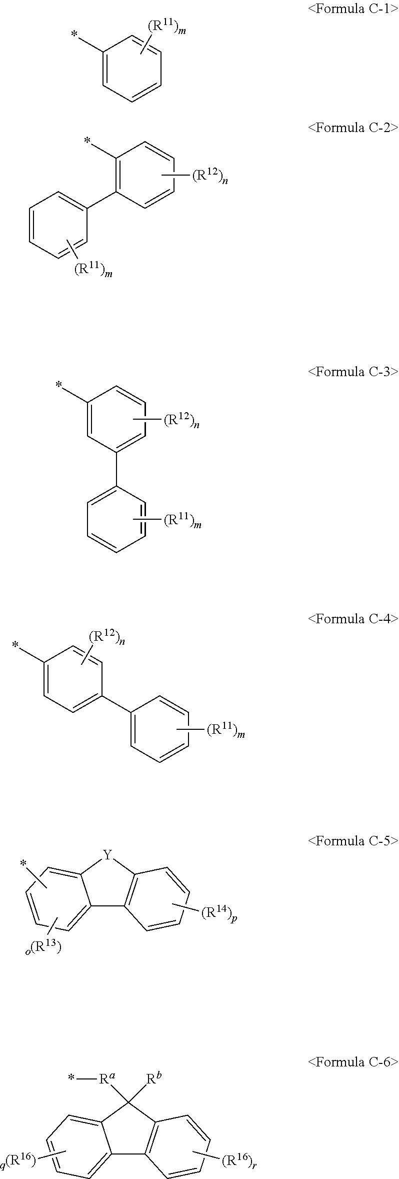

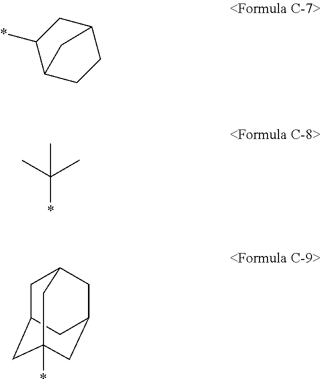

In Formula C-1 to Formula C-9, each symbol can be defined as follows.

Y is O, S, C(R²¹)(R²²) or N(R²³).

R¹¹ to R¹⁶, R²¹, R²², R^{b} are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and adjacent groups may be bonded to each other to form a ring, and R²¹ and R²² may be bonded to each other to form a ring.

A ring formed by adjacent groups may be selected from the group consisting of a C₆-C₆₀ aromatic ring group, a fluorenyl group, a C₂-C₆₀ heterocyclic group containing at least one heteroatom of O, N, S, Si and P, and a C₆-C₆₀ aliphatic ring.

When an aromatic ring is formed by adjacent groups, the aromatic ring may be, for example, a C₆-C₂₀, a C₆-C₁₈, a C₆-C₁₆, a C₆-C₁₄, a C₆-C₁₃, a C₆-C₁₂, a C₆-C₁₀, a C₆, a C₁₀, a C₁₂, a C₁₄, a C₁₅, a C₁₆, or a C₁₈ aromatic ring, specifically, an aryl ring such as benzene, naphthalene, anthracene, phenanthrene, pyrene, etc.

When R²¹ and R²² combine with each other to form a ring, a spiro compound can be formed.

R^{a} is selected from the group consisting of a single bond, a C₁-C₂₀ alkylene group, a C₂-C₂₀ alkenylene group, a C₆-C₃₀ arylene group, a fluorenylene group, a C₂-C₃₀ heterocyclic group containing at least one heteroatom of O, N, S, Si and P, and a C₃-C₃₀ aliphatic ring group.

In addition, and R^{a} and R^{b} may be bonded to each other to form a ring. When R^{a} and R^{b} combine with each other to form a ring, a spiro compound can be formed.

m is an integer of 0 to 5, n, p, q and r are each an integer of 0 to 4, o is an integer of 0 to 3, and when each of these is an integer of 2 or more, each of Riis, each of R¹²s, each of R¹³s, each of R¹⁴s, each of R¹⁵s, each of R¹⁶s are the same as or different from each other.

R²³ is selected from the group consisting of a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group.

Specifically, the compound represented by Formula 1 may be one of the following compounds, but there is no limitation thereto.

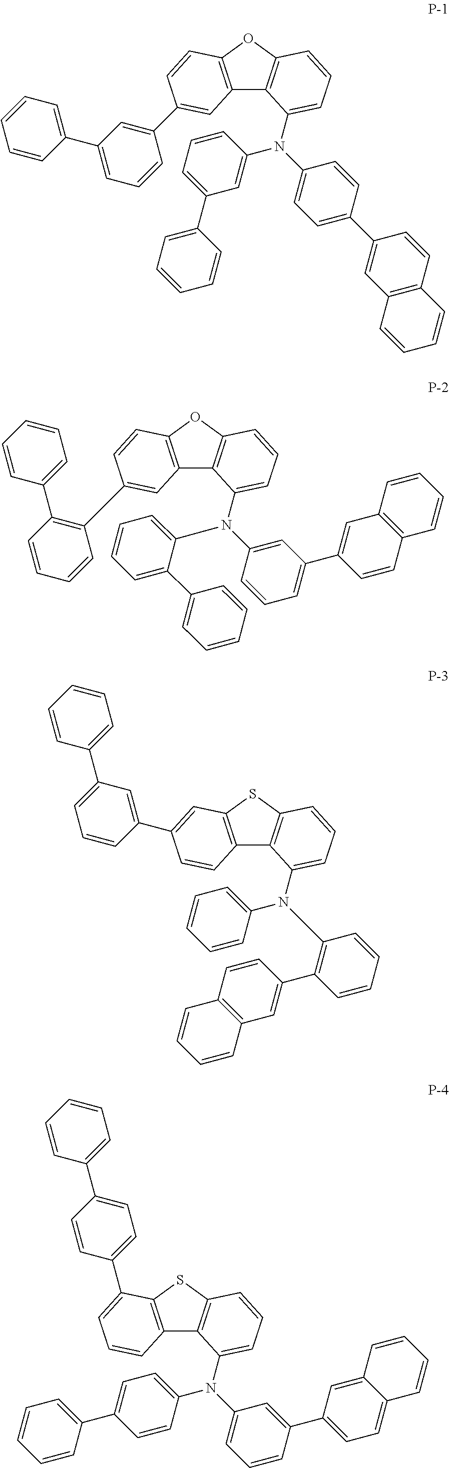 [SMILES: c1ccc(-c2cccc(-c3ccc4oc5cccc(N(c6ccc(-c7ccc8ccccc8c7)cc6)c6cccc(-c7ccccc7)c6)c5c4c3)c2)cc1 ; c1ccc(-c2ccccc2-c2ccc3oc4cccc(N(c5cccc(-c6ccc7ccccc7c6)c5)c5ccccc5-c5ccccc5)c4c3c2)cc1 ; c1ccc(-c2cccc(-c3ccc4c(c3)sc3cccc(N(c5ccccc5)c5ccccc5-c5ccc6ccccc6c5)c34)c2)cc1 ; c1ccc(-c2ccc(-c3cccc4c3sc3cccc(N(c5ccc(-c6ccccc6)cc5)c5cccc(-c6ccc7ccccc7c6)c5)c34)cc2)cc1]

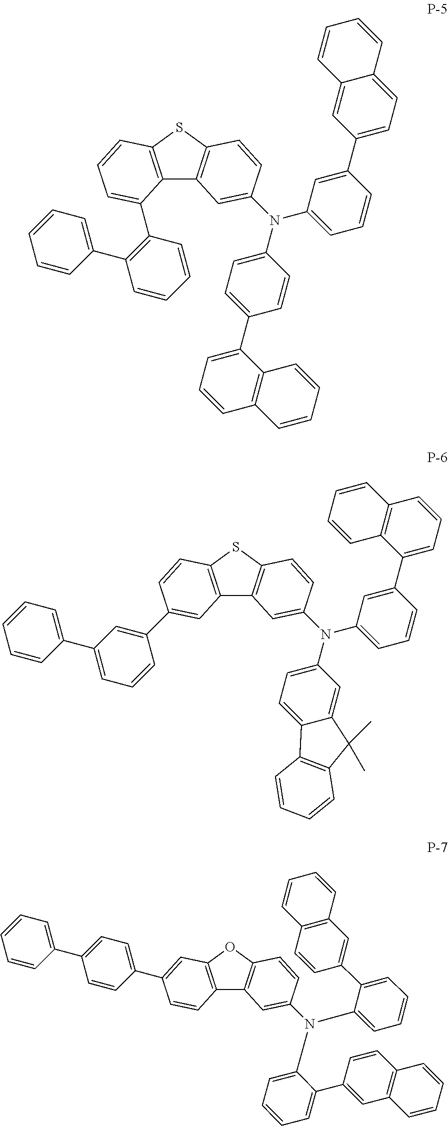 [SMILES: c1ccc(-c2ccccc2-c2cccc3sc4ccc(N(c5ccc(-c6cccc7ccccc67)cc5)c5cccc(-c6ccc7ccccc7c6)c5)cc4c23)cc1 ; CC1(C)c2ccccc2-c2ccc(N(c3cccc(-c4cccc5ccccc45)c3)c3ccc4sc5ccc(-c6cccc(-c7ccccc7)c6)cc5c4c3)cc21 ; c1ccc(-c2ccc(-c3ccc4c(c3)oc3ccc(N(c5ccccc5-c5ccc6ccccc6c5)c5ccccc5-c5ccc6ccccc6c5)cc34)cc2)cc1]

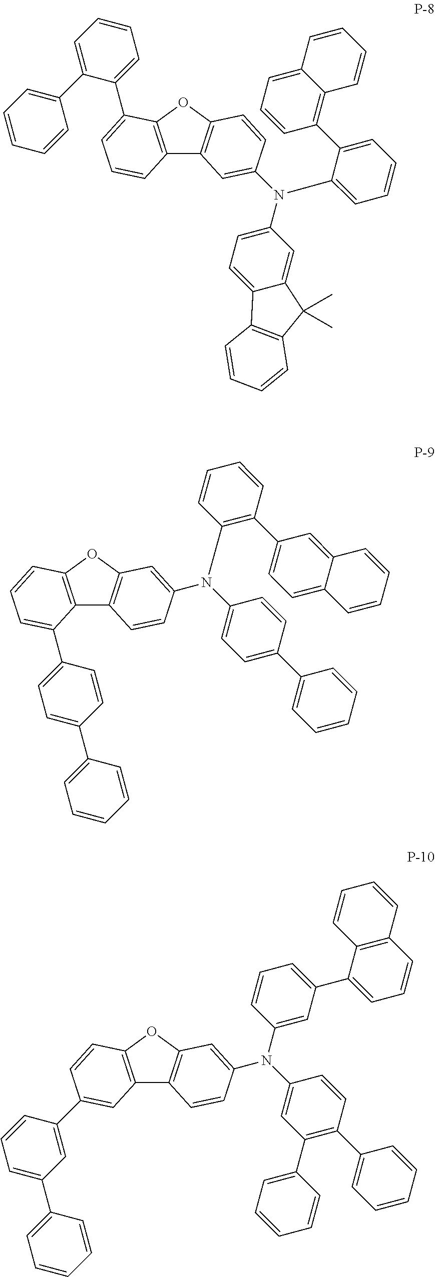 [SMILES: CC1(C)c2ccccc2-c2ccc(N(c3ccc4oc5c(-c6ccccc6-c6ccccc6)cccc5c4c3)c3ccccc3-c3cccc4ccccc34)cc21 ; c1ccc(-c2ccc(-c3cccc4oc5cc(N(c6ccc(-c7ccccc7)cc6)c6ccccc6-c6ccc7ccccc7c6)ccc5c34)cc2)cc1 ; c1ccc(-c2cccc(-c3ccc4oc5cc(N(c6cccc(-c7cccc8ccccc78)c6)c6ccc(-c7ccccc7)c(-c7ccccc7)c6)ccc5c4c3)c2)cc1]

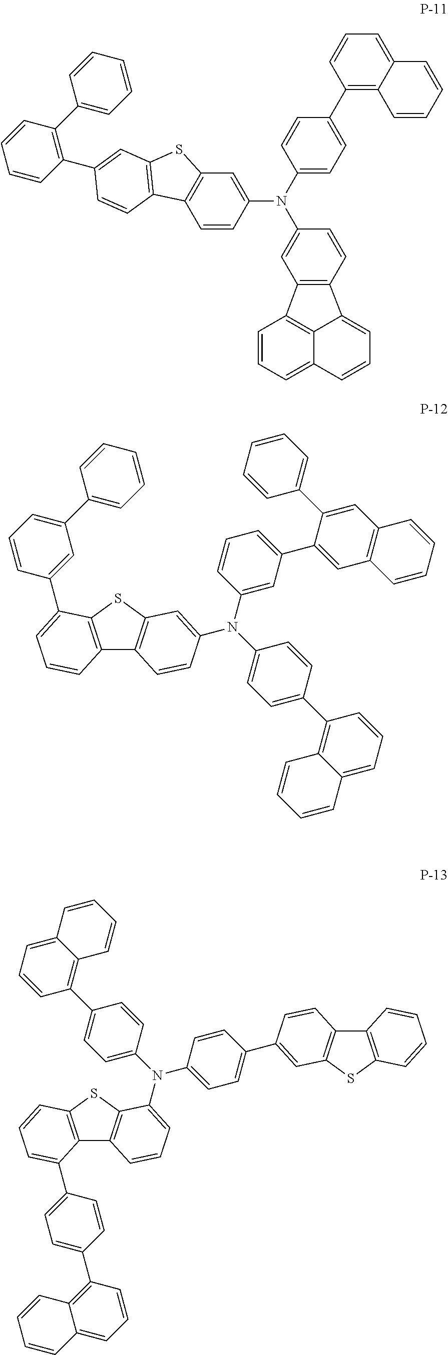 [SMILES: c1ccc(-c2ccccc2-c2ccc3c(c2)sc2cc(N(c4ccc(-c5cccc6ccccc56)cc4)c4ccc5c(c4)-c4cccc6cccc-5c46)ccc23)cc1 ; c1ccc(-c2cccc(-c3cccc4c3sc3cc(N(c5ccc(-c6cccc7ccccc67)cc5)c5cccc(-c6cc7ccccc7cc6-c6ccccc6)c5)ccc34)c2)cc1 ; c1ccc2c(-c3ccc(-c4cccc5sc6c(N(c7ccc(-c8ccc9c(c8)sc8ccccc89)cc7)c7ccc(-c8cccc9ccccc89)cc7)cccc6c45)cc3)cccc2c1]

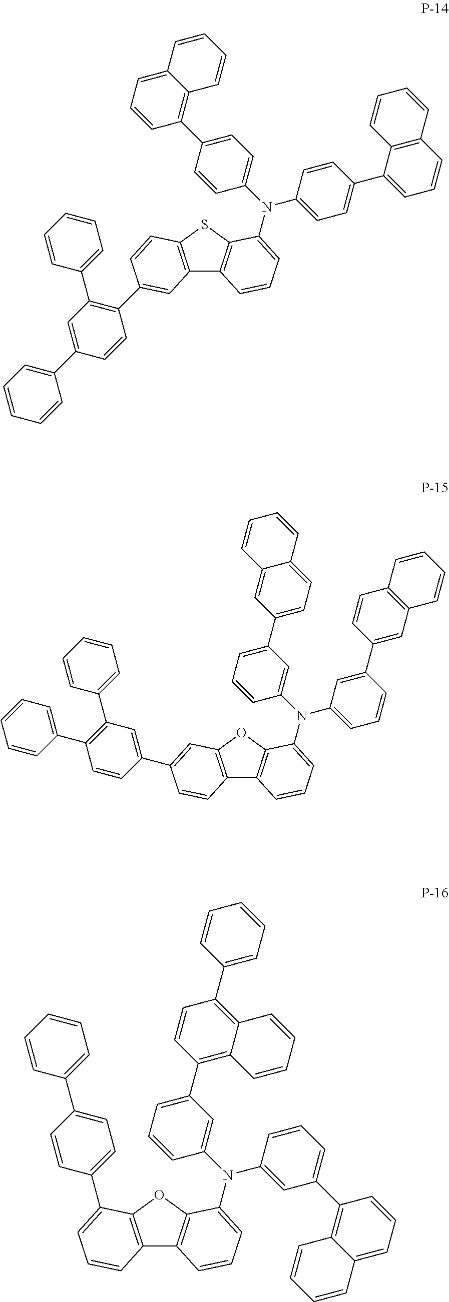 [SMILES: c1ccc(-c2ccc(-c3ccc4sc5c(N(c6ccc(-c7cccc8ccccc78)cc6)c6ccc(-c7cccc8ccccc78)cc6)cccc5c4c3)c(-c3ccccc3)c2)cc1 ; c1ccc(-c2ccc(-c3ccc4c(c3)oc3c(N(c5cccc(-c6ccc7ccccc7c6)c5)c5cccc(-c6ccc7ccccc7c6)c5)cccc34)cc2-c2ccccc2)cc1 ; c1ccc(-c2ccc(-c3cccc4c3oc3c(N(c5cccc(-c6cccc7ccccc67)c5)c5cccc(-c6ccc(-c7ccccc7)c7ccccc67)c5)cccc34)cc2)cc1]

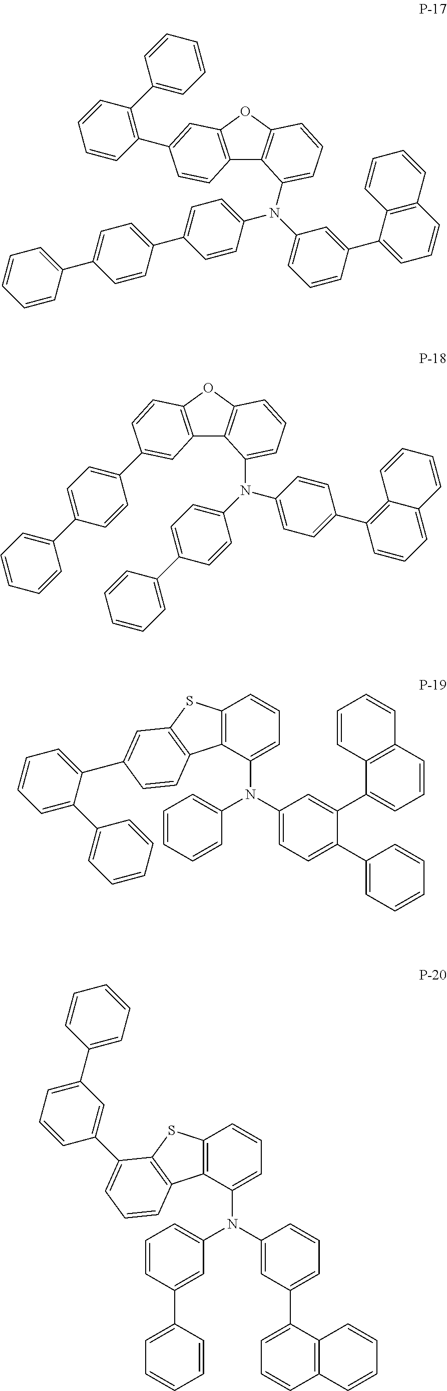 [SMILES: c1ccc(-c2ccc(-c3ccc(N(c4cccc(-c5cccc6ccccc56)c4)c4cccc5oc6cc(-c7ccccc7-c7ccccc7)ccc6c45)cc3)cc2)cc1 ; c1ccc(-c2ccc(-c3ccc4oc5cccc(N(c6ccc(-c7ccccc7)cc6)c6ccc(-c7cccc8ccccc78)cc6)c5c4c3)cc2)cc1 ; c1ccc(-c2ccccc2-c2ccc3c(c2)sc2cccc(N(c4ccccc4)c4ccc(-c5ccccc5)c(-c5cccc6ccccc56)c4)c23)cc1 ; c1ccc(-c2cccc(-c3cccc4c3sc3cccc(N(c5cccc(-c6ccccc6)c5)c5cccc(-c6cccc7ccccc67)c5)c34)c2)cc1]

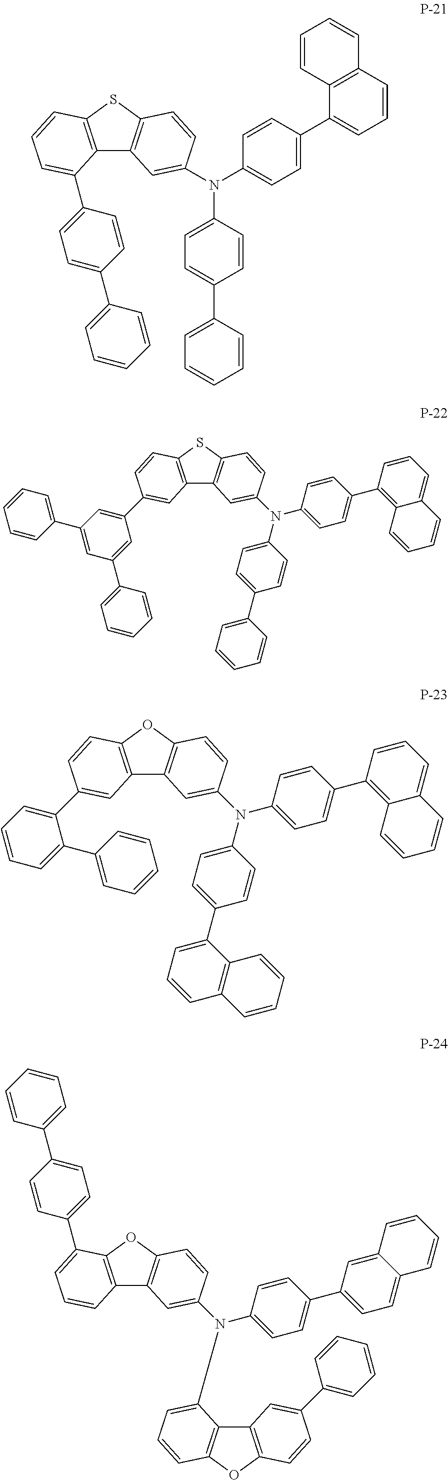 [SMILES: c1ccc(-c2ccc(-c3cccc4sc5ccc(N(c6ccc(-c7ccccc7)cc6)c6ccc(-c7cccc8ccccc78)cc6)cc5c34)cc2)cc1 ; c1ccc(-c2ccc(N(c3ccc(-c4cccc5ccccc45)cc3)c3ccc4sc5ccc(-c6cc(-c7ccccc7)cc(-c7ccccc7)c6)cc5c4c3)cc2)cc1 ; c1ccc(-c2ccccc2-c2ccc3oc4ccc(N(c5ccc(-c6cccc7ccccc67)cc5)c5ccc(-c6cccc7ccccc67)cc5)cc4c3c2)cc1 ; c1ccc(-c2ccc(-c3cccc4c3oc3ccc(N(c5ccc(-c6ccc7ccccc7c6)cc5)c5cccc6oc7ccc(-c8ccccc8)cc7c56)cc34)cc2)cc1]

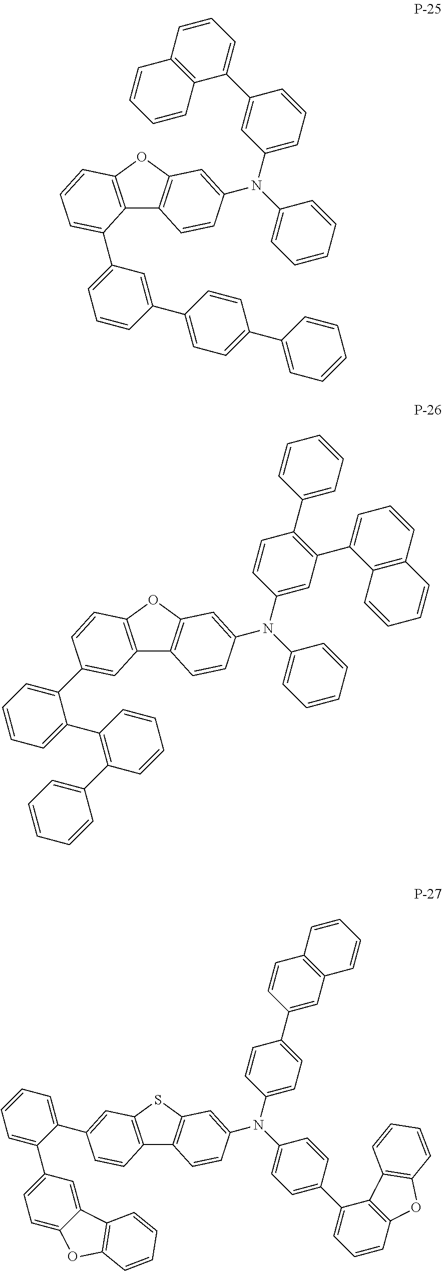 [SMILES: c1ccc(-c2ccc(-c3cccc(-c4cccc5oc6cc(N(c7ccccc7)c7cccc(-c8cccc9ccccc89)c7)ccc6c45)c3)cc2)cc1 ; c1ccc(-c2ccccc2-c2ccccc2-c2ccc3oc4cc(N(c5ccccc5)c5ccc(-c6ccccc6)c(-c6cccc7ccccc67)c5)ccc4c3c2)cc1 ; c1ccc(-c2ccc3oc4ccccc4c3c2)c(-c2ccc3c(c2)sc2cc(N(c4ccc(-c5ccc6ccccc6c5)cc4)c4ccc(-c5cccc6oc7ccccc7c56)cc4)ccc23)c1]

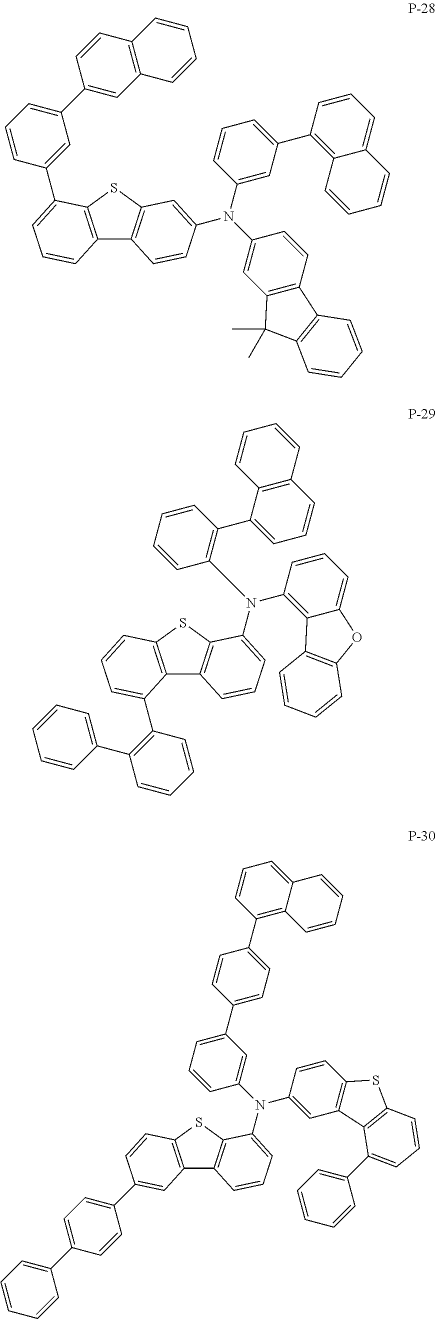 [SMILES: CC1(C)c2ccccc2-c2ccc(N(c3cccc(-c4cccc5ccccc45)c3)c3ccc4c(c3)sc3c(-c5cccc(-c6ccc7ccccc7c6)c5)cccc34)cc21 ; c1ccc(-c2ccccc2-c2cccc3sc4c(N(c5ccccc5-c5cccc6ccccc56)c5cccc6oc7ccccc7c56)cccc4c23)cc1 ; c1ccc(-c2ccc(-c3ccc4sc5c(N(c6cccc(-c7ccc(-c8cccc9ccccc89)cc7)c6)c6ccc7sc8cccc(-c9ccccc9)c8c7c6)cccc5c4c3)cc2)cc1]

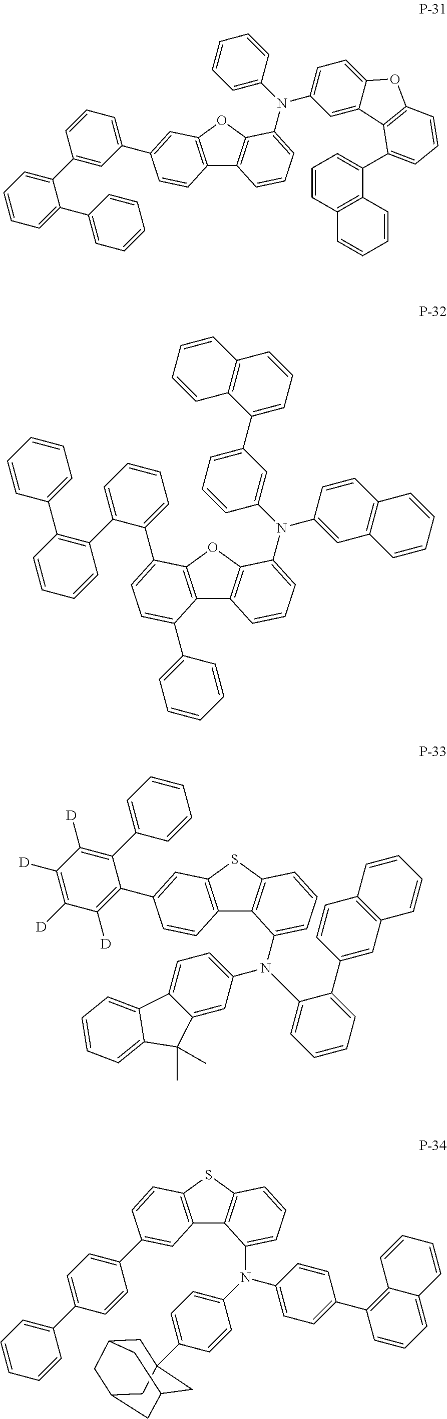 [SMILES: c1ccc(-c2ccccc2-c2cccc(-c3ccc4c(c3)oc3c(N(c5ccccc5)c5ccc6oc7cccc(-c8cccc9ccccc89)c7c6c5)cccc34)c2)cc1 ; c1ccc(-c2ccccc2-c2ccccc2-c2ccc(-c3ccccc3)c3c2oc2c(N(c4cccc(-c5cccc6ccccc56)c4)c4ccc5ccccc5c4)cccc23)cc1 ; [2H]c1c([2H])c([2H])c(-c2ccc3c(c2)sc2cccc(N(c4ccc5c(c4)C(C)(C)c4ccccc4-5)c4ccccc4-c4ccc5ccccc5c4)c23)c(-c2ccccc2)c1[2H] ; c1ccc(-c2ccc(-c3ccc4sc5cccc(N(c6ccc(-c7cccc8ccccc78)cc6)c6ccc(C78CC9CC(CC(C9)C7)C8)cc6)c5c4c3)cc2)cc1]

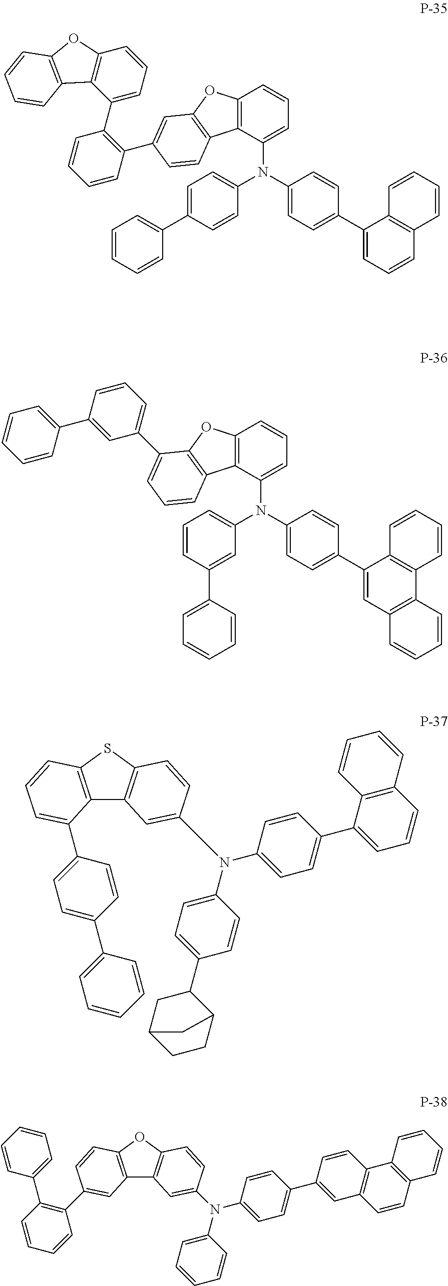 [SMILES: c1ccc(-c2ccc(N(c3ccc(-c4cccc5ccccc45)cc3)c3cccc4oc5cc(-c6ccccc6-c6cccc7oc8ccccc8c67)ccc5c34)cc2)cc1 ; c1ccc(-c2cccc(-c3cccc4c3oc3cccc(N(c5ccc(-c6cc7ccccc7c7ccccc67)cc5)c5cccc(-c6ccccc6)c5)c34)c2)cc1 ; c1ccc(-c2ccc(-c3cccc4sc5ccc(N(c6ccc(-c7cccc8ccccc78)cc6)c6ccc(C7CC8CCC7C8)cc6)cc5c34)cc2)cc1 ; c1ccc(-c2ccccc2-c2ccc3oc4ccc(N(c5ccccc5)c5ccc(-c6ccc7c(ccc8ccccc87)c6)cc5)cc4c3c2)cc1]

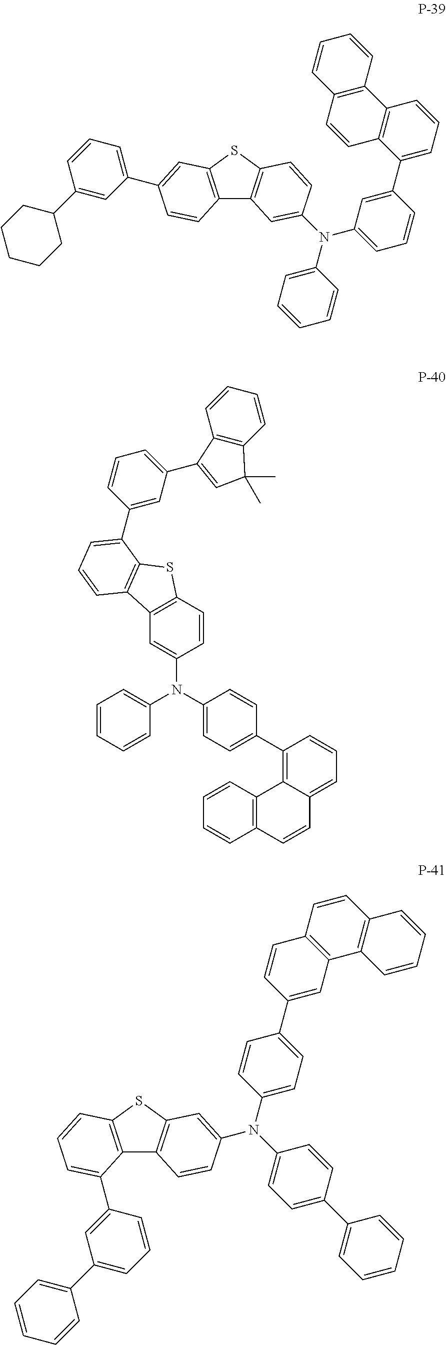 [SMILES: c1ccc(N(c2cccc(-c3cccc4c3ccc3ccccc34)c2)c2ccc3sc4cc(-c5cccc(C6CCCCC6)c5)ccc4c3c2)cc1 ; CC1(C)C=C(c2cccc(-c3cccc4c3sc3ccc(N(c5ccccc5)c5ccc(-c6cccc7ccc8ccccc8c67)cc5)cc34)c2)c2ccccc21 ; c1ccc(-c2ccc(N(c3ccc(-c4ccc5ccc6ccccc6c5c4)cc3)c3ccc4c(c3)sc3cccc(-c5cccc(-c6ccccc6)c5)c34)cc2)cc1]

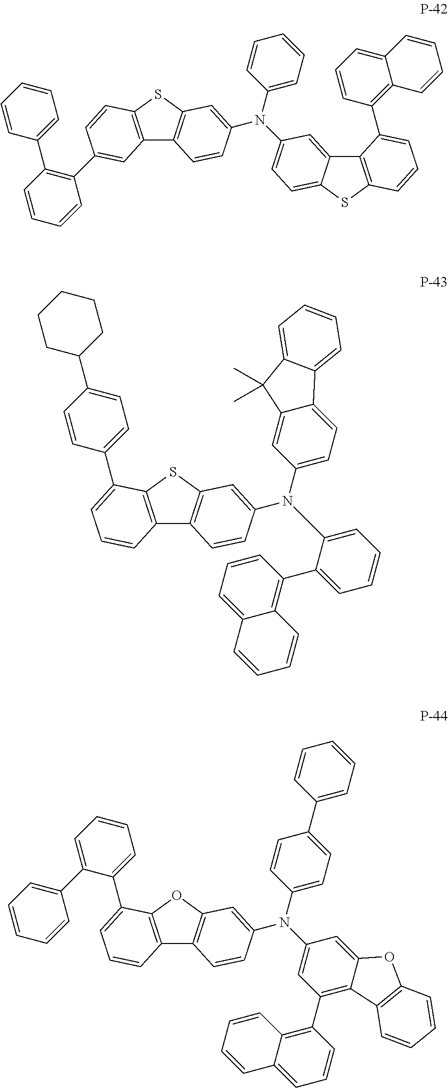 [SMILES: c1ccc(-c2ccccc2-c2ccc3sc4cc(N(c5ccccc5)c5ccc6sc7cccc(-c8cccc9ccccc89)c7c6c5)ccc4c3c2)cc1 ; CC1(C)c2ccccc2-c2ccc(N(c3ccc4c(c3)sc3c(-c5ccc(C6CCCCC6)cc5)cccc34)c3ccccc3-c3cccc4ccccc34)cc21 ; c1ccc(-c2ccc(N(c3ccc4c(c3)oc3c(-c5ccccc5-c5ccccc5)cccc34)c3cc(-c4cccc5ccccc45)c4c(c3)oc3ccccc34)cc2)cc1]

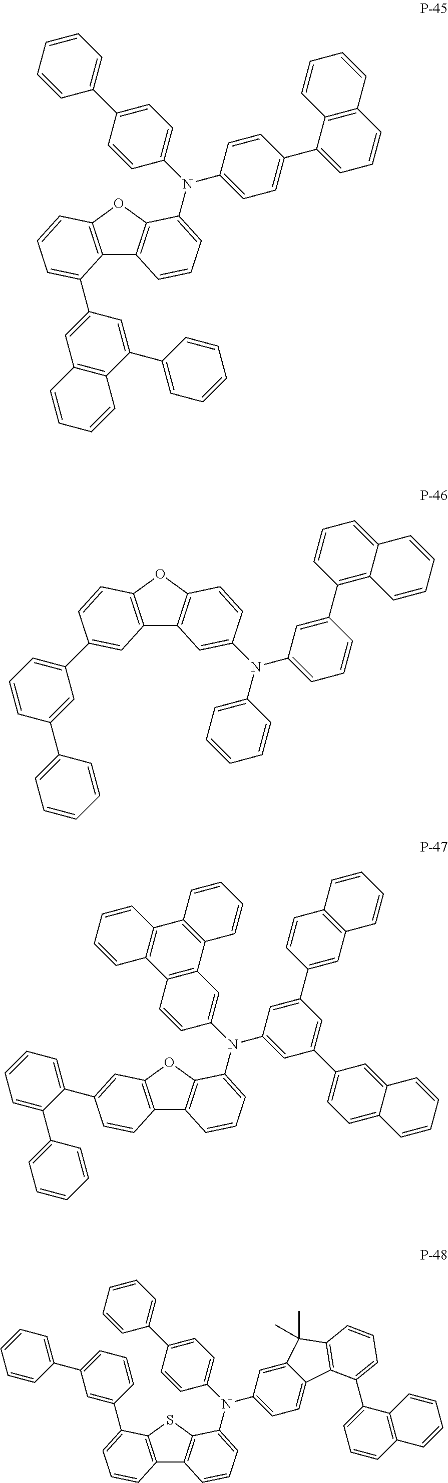 [SMILES: c1ccc(-c2ccc(N(c3ccc(-c4cccc5ccccc45)cc3)c3cccc4c3oc3cccc(-c5cc(-c6ccccc6)c6ccccc6c5)c34)cc2)cc1 ; c1ccc(-c2cccc(-c3ccc4oc5ccc(N(c6ccccc6)c6cccc(-c7cccc8ccccc78)c6)cc5c4c3)c2)cc1 ; c1ccc(-c2ccccc2-c2ccc3c(c2)oc2c(N(c4cc(-c5ccc6ccccc6c5)cc(-c5ccc6ccccc6c5)c4)c4ccc5c6ccccc6c6ccccc6c5c4)cccc23)cc1 ; CC1(C)c2cc(N(c3ccc(-c4ccccc4)cc3)c3cccc4c3sc3c(-c5cccc(-c6ccccc6)c5)cccc34)ccc2-c2c(-c3cccc4ccccc34)cccc21]

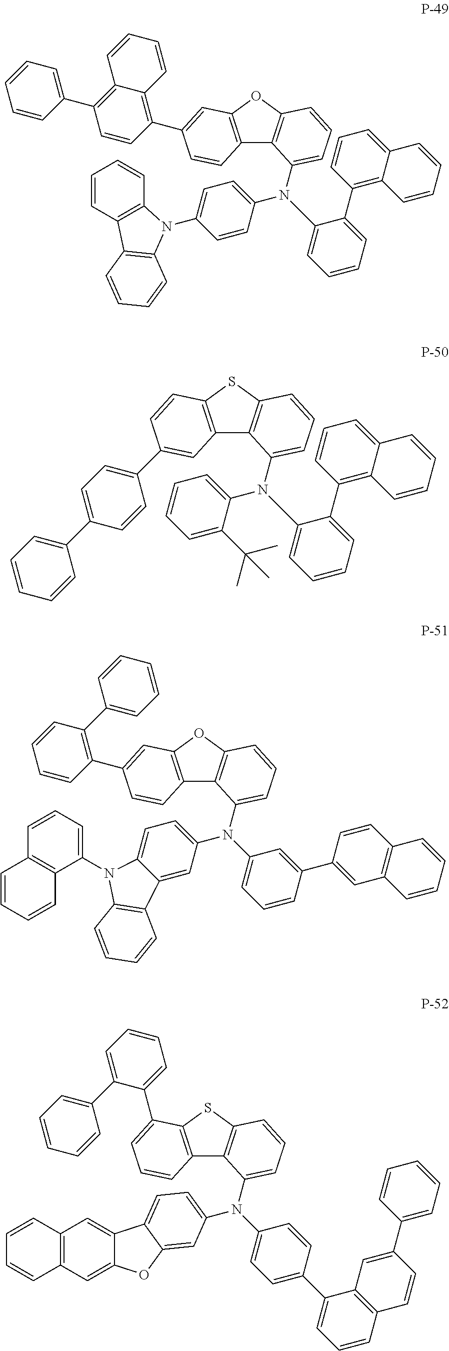 [SMILES: c1ccc(-c2ccc(-c3ccc4c(c3)oc3cccc(N(c5ccc(-n6c7ccccc7c7ccccc76)cc5)c5ccccc5-c5cccc6ccccc56)c34)c3ccccc23)cc1 ; CC(C)(C)c1ccccc1N(c1ccccc1-c1cccc2ccccc12)c1cccc2sc3ccc(-c4ccc(-c5ccccc5)cc4)cc3c12 ; c1ccc(-c2ccccc2-c2ccc3c(c2)oc2cccc(N(c4cccc(-c5ccc6ccccc6c5)c4)c4ccc5c(c4)c4ccccc4n5-c4cccc5ccccc45)c23)cc1 ; c1ccc(-c2ccc3cccc(-c4ccc(N(c5ccc6c(c5)oc5cc7ccccc7cc56)c5cccc6sc7c(-c8ccccc8-c8ccccc8)cccc7c56)cc4)c3c2)cc1]

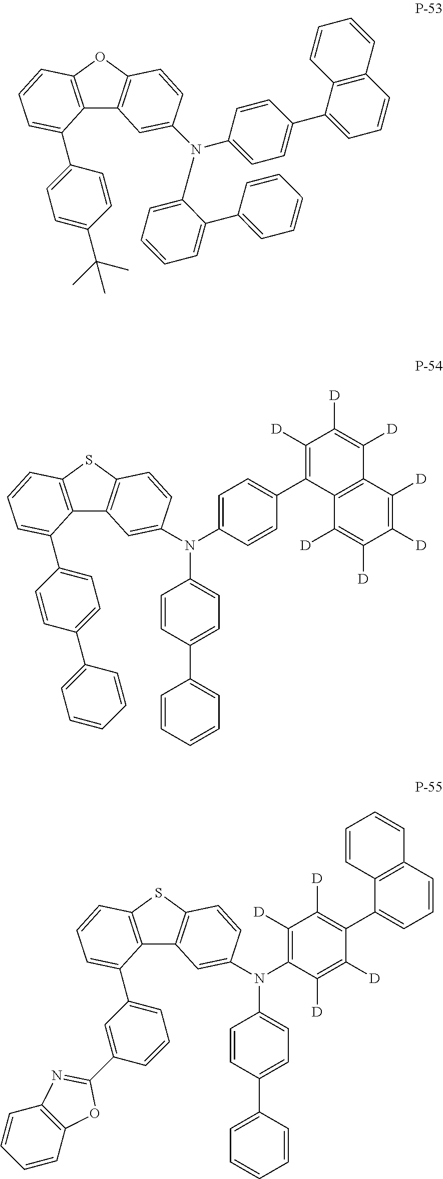 [SMILES: CC(C)(C)c1ccc(-c2cccc3oc4ccc(N(c5ccc(-c6cccc7ccccc67)cc5)c5ccccc5-c5ccccc5)cc4c23)cc1 ; [2H]c1c([2H])c([2H])c2c(-c3ccc(N(c4ccc(-c5ccccc5)cc4)c4ccc5sc6cccc(-c7ccc(-c8ccccc8)cc7)c6c5c4)cc3)c([2H])c([2H])c([2H])c2c1[2H] ; [2H]c1c([2H])c(N(c2ccc(-c3ccccc3)cc2)c2ccc3sc4cccc(-c5cccc(-c6nc7ccccc7o6)c5)c4c3c2)c([2H])c([2H])c1-c1cccc2ccccc12]

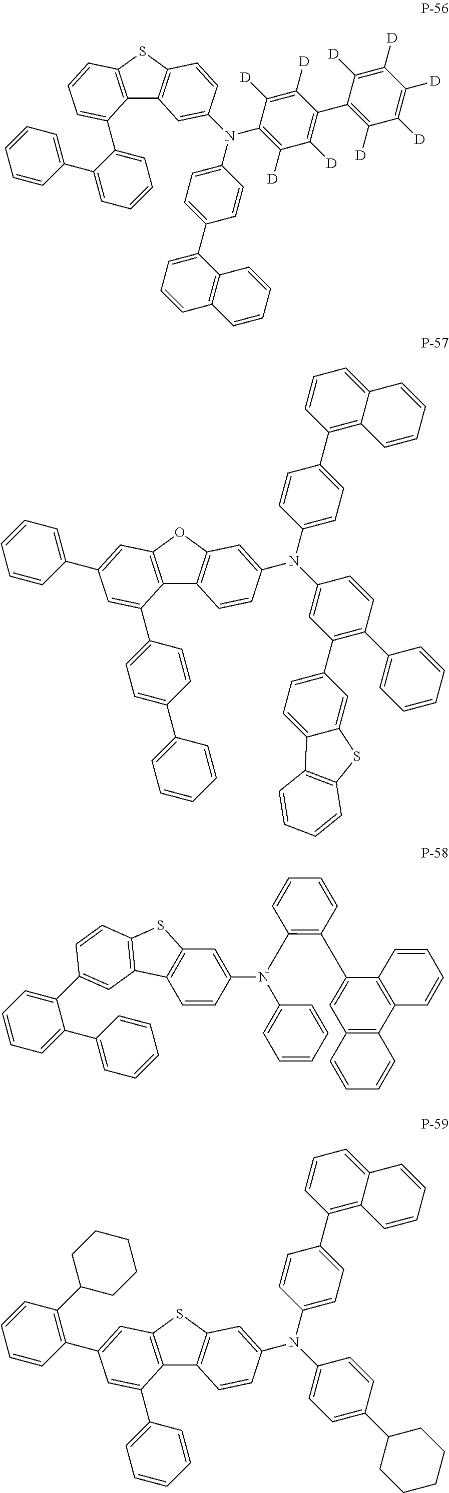

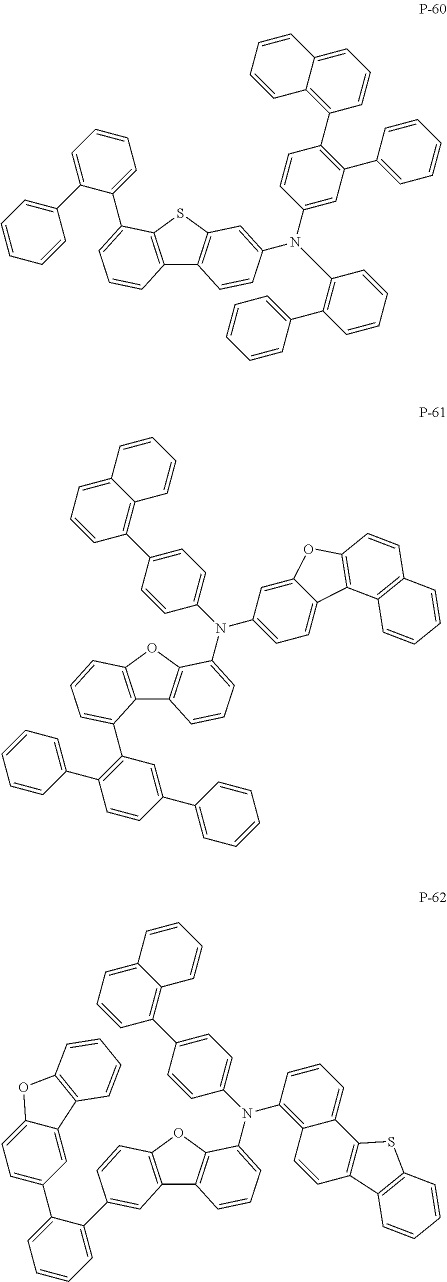 [SMILES: c1ccc(-c2cc(N(c3ccc4c(c3)sc3c(-c5ccccc5-c5ccccc5)cccc34)c3ccccc3-c3ccccc3)ccc2-c2cccc3ccccc23)cc1 ; c1ccc(-c2ccc(-c3ccccc3)c(-c3cccc4oc5c(N(c6ccc(-c7cccc8ccccc78)cc6)c6ccc7c(c6)oc6ccc8ccccc8c67)cccc5c34)c2)cc1 ; c1ccc(-c2ccc3oc4c(N(c5ccc(-c6cccc7ccccc67)cc5)c5cccc6c5ccc5c7ccccc7sc65)cccc4c3c2)c(-c2ccc3oc4ccccc4c3c2)c1]

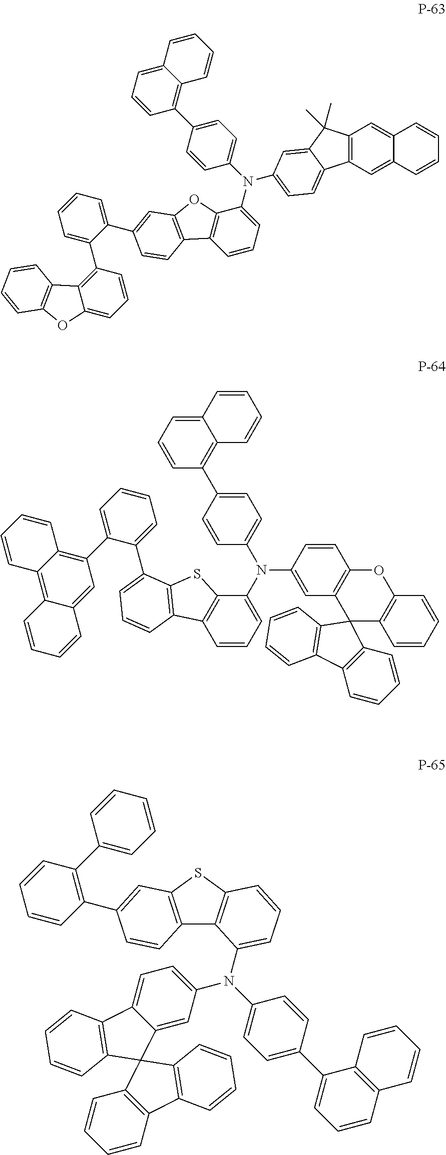 [SMILES: CC1(C)c2cc(N(c3ccc(-c4cccc5ccccc45)cc3)c3cccc4c3oc3cc(-c5ccccc5-c5cccc6oc7ccccc7c56)ccc34)ccc2-c2cc3ccccc3cc21 ; c1ccc2c(c1)Oc1ccc(N(c3ccc(-c4cccc5ccccc45)cc3)c3cccc4c3sc3c(-c5ccccc5-c5cc6ccccc6c6ccccc56)cccc34)cc1C21c2ccccc2-c2ccccc21 ; c1ccc(-c2ccccc2-c2ccc3c(c2)sc2cccc(N(c4ccc(-c5cccc6ccccc56)cc4)c4ccc5c(c4)C4(c6ccccc6-c6ccccc64)c4ccccc4-5)c23)cc1]

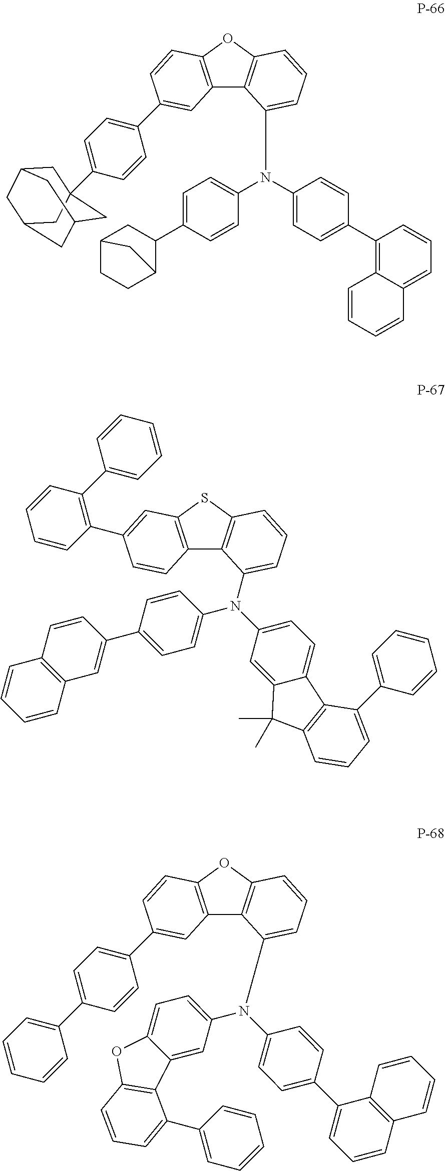 [SMILES: c1ccc2c(-c3ccc(N(c4ccc(C5CC6CCC5C6)cc4)c4cccc5oc6ccc(-c7ccc(C89CC%10CC(CC(C%10)C8)C9)cc7)cc6c45)cc3)cccc2c1 ; CC1(C)c2cc(N(c3ccc(-c4ccc5ccccc5c4)cc3)c3cccc4sc5cc(-c6ccccc6-c6ccccc6)ccc5c34)ccc2-c2c(-c3ccccc3)cccc21 ; c1ccc(-c2ccc(-c3ccc4oc5cccc(N(c6ccc(-c7cccc8ccccc78)cc6)c6ccc7oc8cccc(-c9ccccc9)c8c7c6)c5c4c3)cc2)cc1]

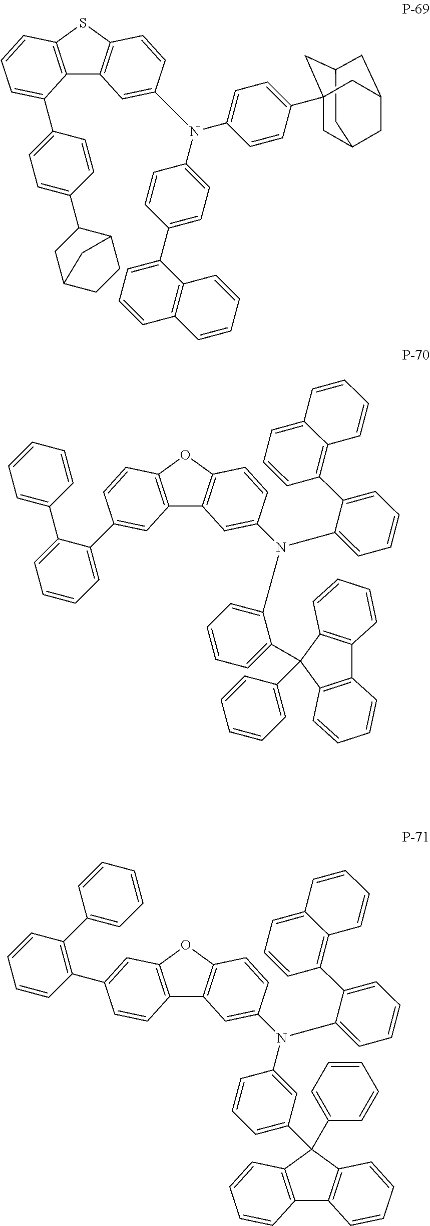 [SMILES: c1ccc2c(-c3ccc(N(c4ccc(C56CC7CC(CC(C7)C5)C6)cc4)c4ccc5sc6cccc(-c7ccc(C8CC9CCC8C9)cc7)c6c5c4)cc3)cccc2c1 ; c1ccc(-c2ccccc2-c2ccc3oc4ccc(N(c5ccccc5-c5cccc6ccccc56)c5ccccc5C5(c6ccccc6)c6ccccc6-c6ccccc65)cc4c3c2)cc1 ; c1ccc(-c2ccccc2-c2ccc3c(c2)oc2ccc(N(c4cccc(C5(c6ccccc6)c6ccccc6-c6ccccc65)c4)c4ccccc4-c4cccc5ccccc45)cc23)cc1]

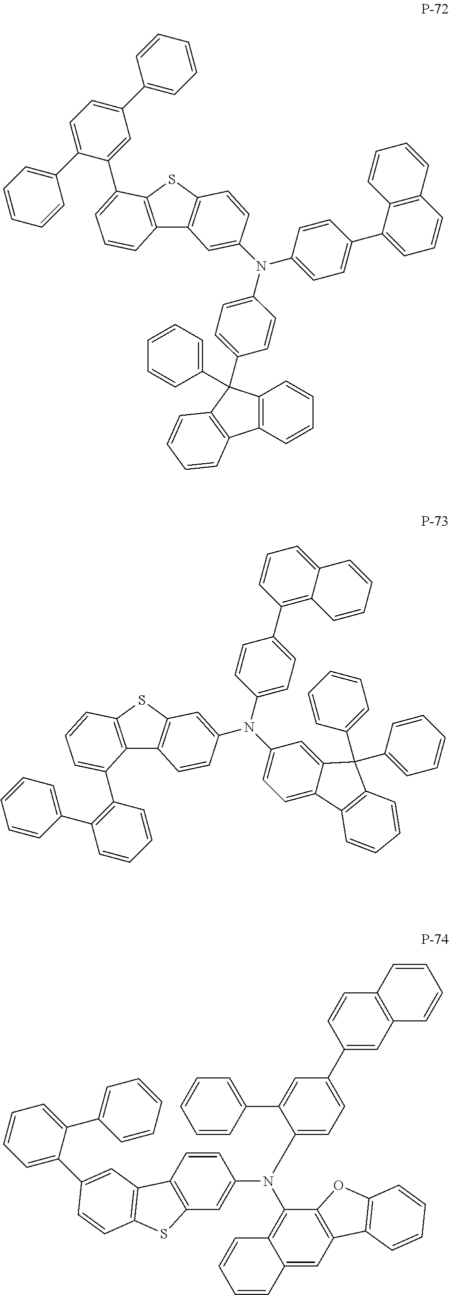 [SMILES: c1ccc(-c2ccc(-c3ccccc3)c(-c3cccc4c3sc3ccc(N(c5ccc(-c6cccc7ccccc67)cc5)c5ccc(C6(c7ccccc7)c7ccccc7-c7ccccc76)cc5)cc34)c2)cc1 ; c1ccc(-c2ccccc2-c2cccc3sc4cc(N(c5ccc(-c6cccc7ccccc67)cc5)c5ccc6c(c5)C(c5ccccc5)(c5ccccc5)c5ccccc5-6)ccc4c23)cc1 ; c1ccc(-c2ccccc2-c2ccc3sc4cc(N(c5ccc(-c6ccc7ccccc7c6)cc5-c5ccccc5)c5c6ccccc6cc6c5oc5ccccc56)ccc4c3c2)cc1]

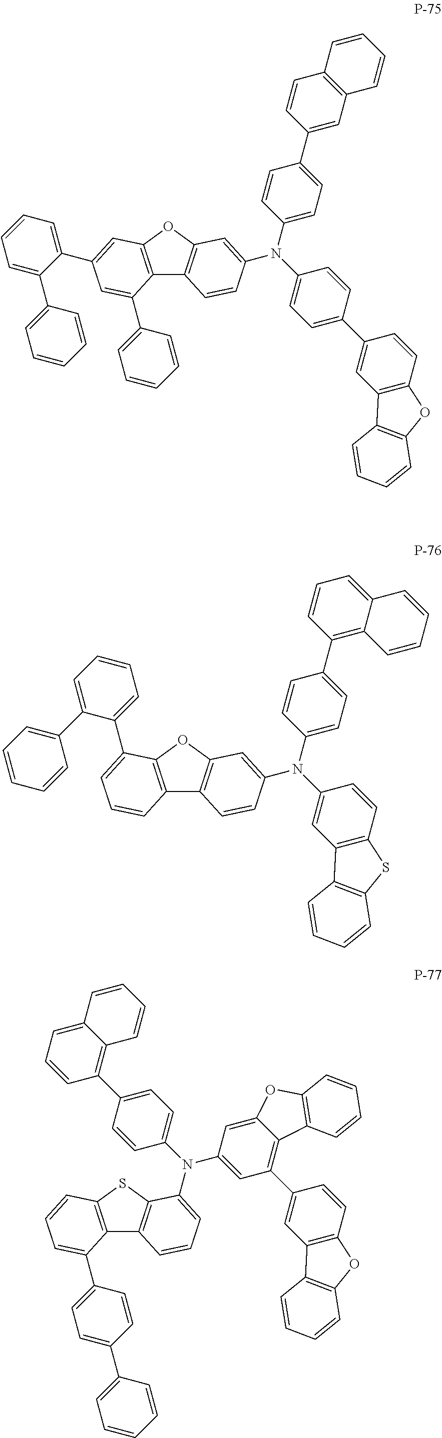 [SMILES: c1ccc(-c2ccccc2-c2cc(-c3ccccc3)c3c(c2)oc2cc(N(c4ccc(-c5ccc6ccccc6c5)cc4)c4ccc(-c5ccc6oc7ccccc7c6c5)cc4)ccc23)cc1 ; c1ccc(-c2ccccc2-c2cccc3c2oc2cc(N(c4ccc(-c5cccc6ccccc56)cc4)c4ccc5sc6ccccc6c5c4)ccc23)cc1 ; c1ccc(-c2ccc(-c3cccc4sc5c(N(c6ccc(-c7cccc8ccccc78)cc6)c6cc(-c7ccc8oc9ccccc9c8c7)c7c(c6)oc6ccccc67)cccc5c34)cc2)cc1]

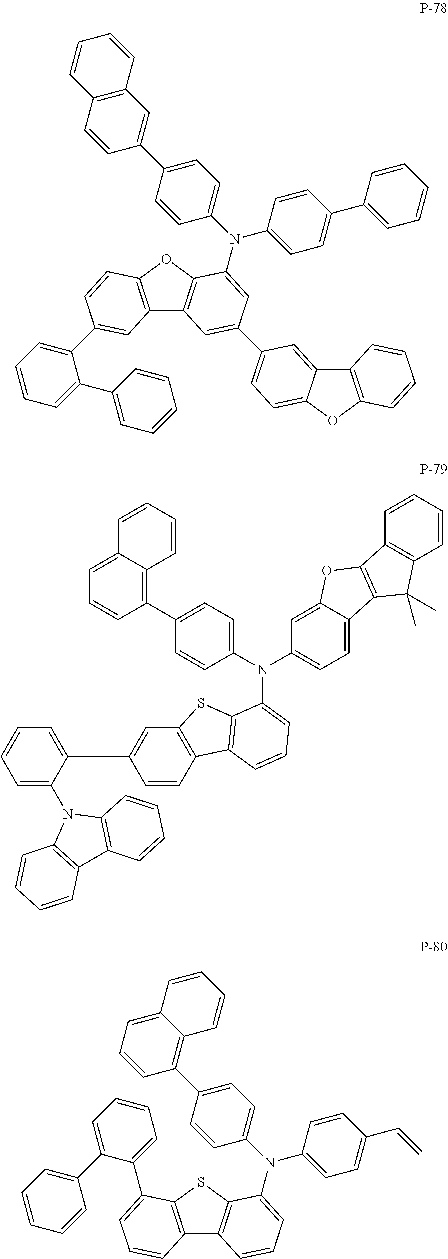 [SMILES: c1ccc(-c2ccc(N(c3ccc(-c4ccc5ccccc5c4)cc3)c3cc(-c4ccc5oc6ccccc6c5c4)cc4c3oc3ccc(-c5ccccc5-c5ccccc5)cc34)cc2)cc1 ; CC1(C)c2ccccc2-c2oc3cc(N(c4ccc(-c5cccc6ccccc56)cc4)c4cccc5c4sc4cc(-c6ccccc6-n6c7ccccc7c7ccccc76)ccc45)ccc3c21 ; C=Cc1ccc(N(c2ccc(-c3cccc4ccccc34)cc2)c2cccc3c2sc2c(-c4ccccc4-c4ccccc4)cccc23)cc1]

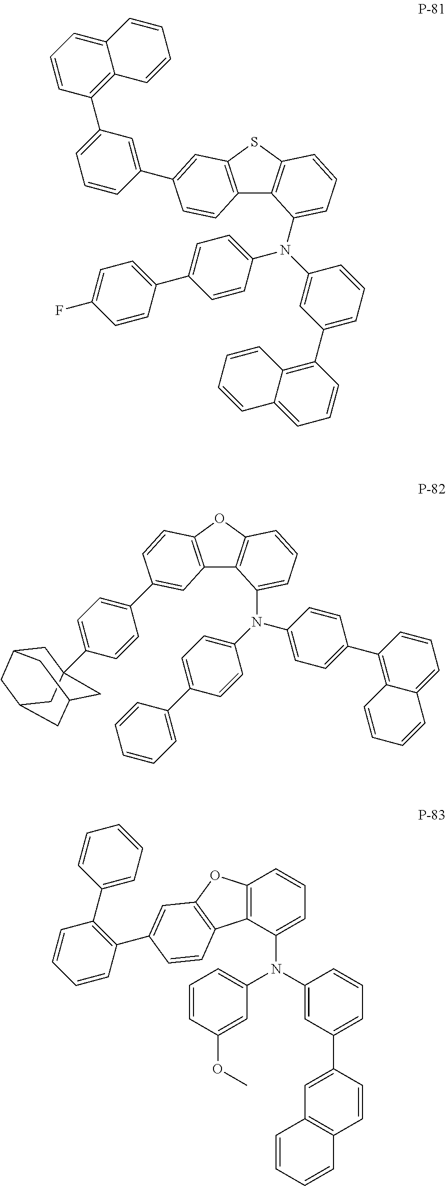 [SMILES: Fc1ccc(-c2ccc(N(c3cccc(-c4cccc5ccccc45)c3)c3cccc4sc5cc(-c6cccc(-c7cccc8ccccc78)c6)ccc5c34)cc2)cc1 ; c1ccc(-c2ccc(N(c3ccc(-c4cccc5ccccc45)cc3)c3cccc4oc5ccc(-c6ccc(C78CC9CC(CC(C9)C7)C8)cc6)cc5c34)cc2)cc1 ; COc1cccc(N(c2cccc(-c3ccc4ccccc4c3)c2)c2cccc3oc4cc(-c5ccccc5-c5ccccc5)ccc4c23)c1]

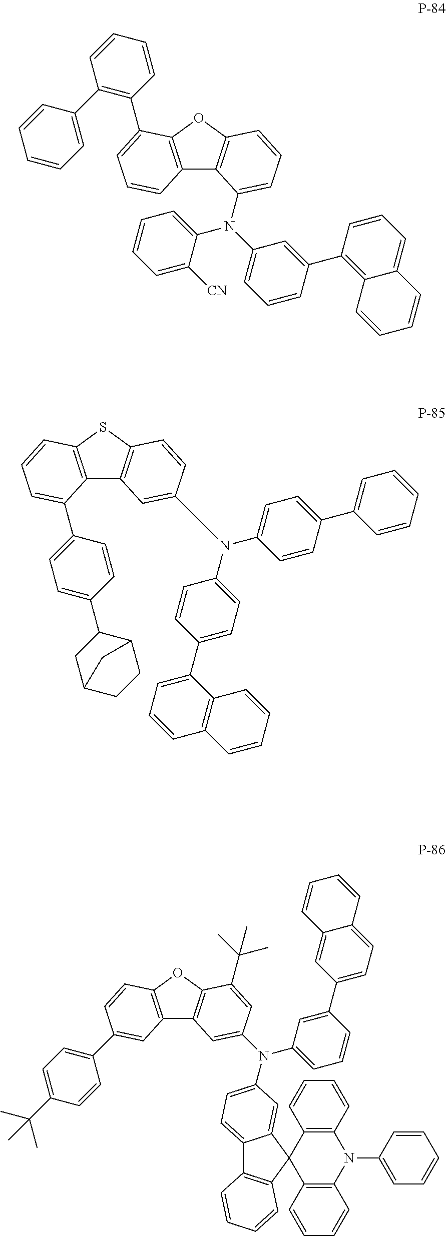

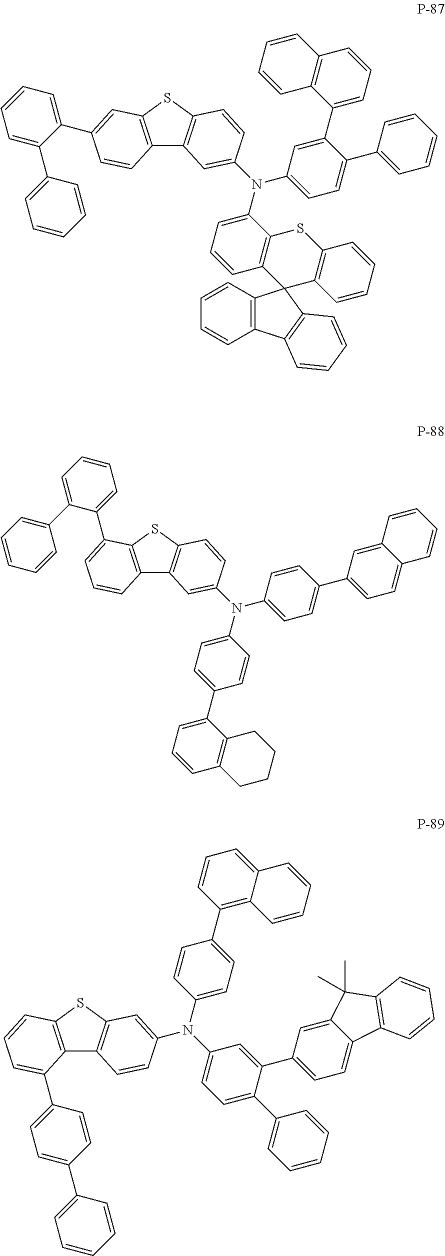 [SMILES: c1ccc(-c2ccccc2-c2ccc3c(c2)sc2ccc(N(c4ccc(-c5ccccc5)c(-c5cccc6ccccc56)c4)c4cccc5c4Sc4ccccc4C54c5ccccc5-c5ccccc54)cc23)cc1 ; c1ccc(-c2ccccc2-c2cccc3c2sc2ccc(N(c4ccc(-c5ccc6ccccc6c5)cc4)c4ccc(-c5cccc6c5CCCC6)cc4)cc23)cc1 ; CC1(C)c2ccccc2-c2ccc(-c3cc(N(c4ccc(-c5cccc6ccccc56)cc4)c4ccc5c(c4)sc4cccc(-c6ccc(-c7ccccc7)cc6)c45)ccc3-c3ccccc3)cc21]

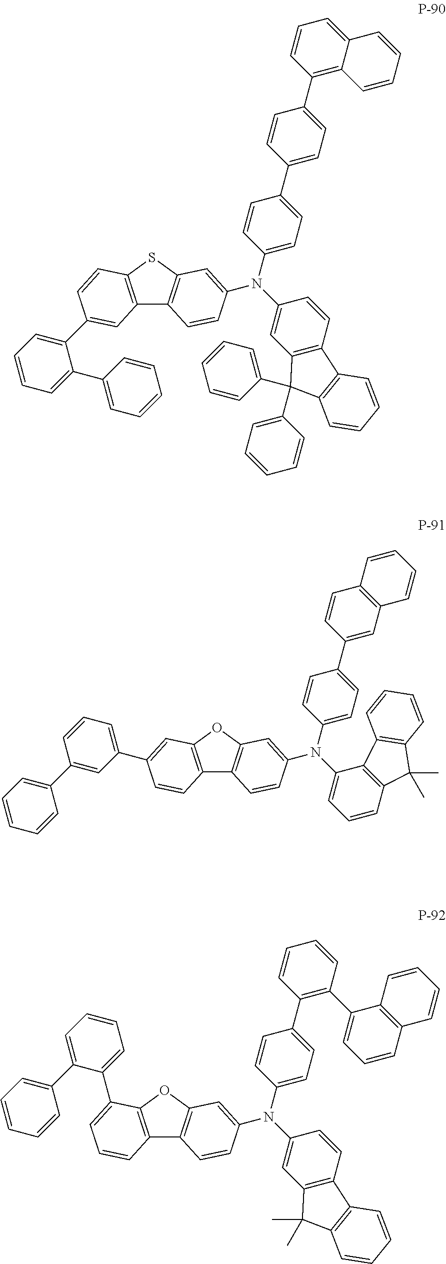 [SMILES: c1ccc(-c2ccccc2-c2ccc3sc4cc(N(c5ccc(-c6ccc(-c7cccc8ccccc78)cc6)cc5)c5ccc6c(c5)C(c5ccccc5)(c5ccccc5)c5ccccc5-6)ccc4c3c2)cc1 ; CC1(C)c2ccccc2-c2c(N(c3ccc(-c4ccc5ccccc5c4)cc3)c3ccc4c(c3)oc3cc(-c5cccc(-c6ccccc6)c5)ccc34)cccc21 ; CC1(C)c2ccccc2-c2ccc(N(c3ccc(-c4ccccc4-c4cccc5ccccc45)cc3)c3ccc4c(c3)oc3c(-c5ccccc5-c5ccccc5)cccc34)cc21]

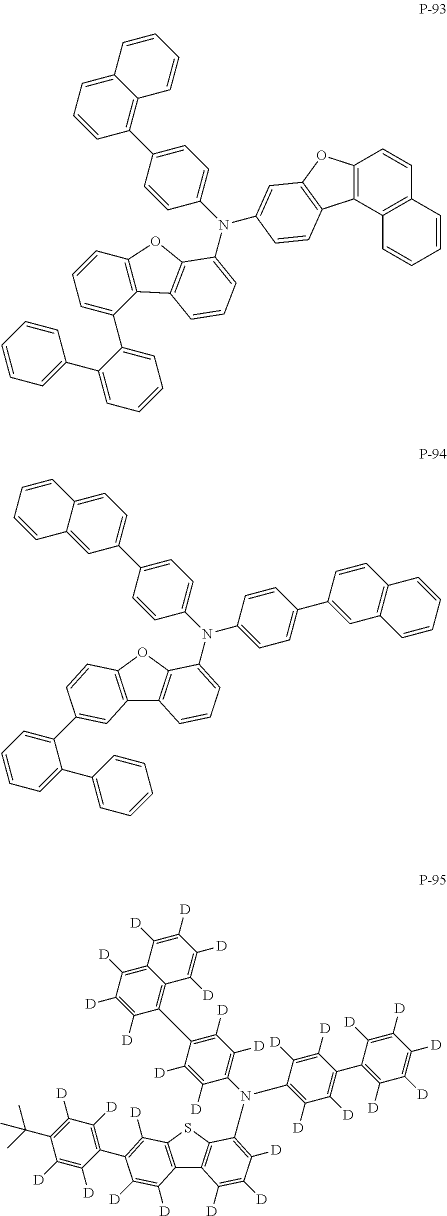 [SMILES: c1ccc(-c2ccccc2-c2cccc3oc4c(N(c5ccc(-c6cccc7ccccc67)cc5)c5ccc6c(c5)oc5ccc7ccccc7c56)cccc4c23)cc1 ; c1ccc(-c2ccccc2-c2ccc3oc4c(N(c5ccc(-c6ccc7ccccc7c6)cc5)c5ccc(-c6ccc7ccccc7c6)cc5)cccc4c3c2)cc1 ; [2H]c1c([2H])c([2H])c(-c2c([2H])c([2H])c(N(c3c([2H])c([2H])c(-c4c([2H])c([2H])c([2H])c5c([2H])c([2H])c([2H])c([2H])c45)c([2H])c3[2H])c3c([2H])c([2H])c([2H])c4c3sc3c([2H])c(-c5c([2H])c([2H])c(C(C)(C)C)c([2H])c5[2H])c([2H])c([2H])c34)c([2H])c2[2H])c([2H])c1[2H]]

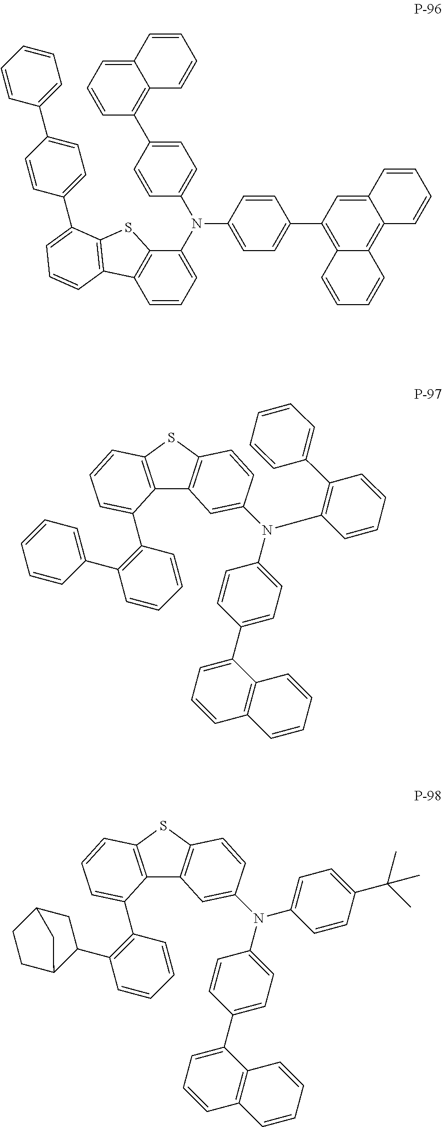 [SMILES: c1ccc(-c2ccc(-c3cccc4c3sc3c(N(c5ccc(-c6cccc7ccccc67)cc5)c5ccc(-c6cc7ccccc7c7ccccc67)cc5)cccc34)cc2)cc1 ; c1ccc(-c2ccccc2-c2cccc3sc4ccc(N(c5ccc(-c6cccc7ccccc67)cc5)c5ccccc5-c5ccccc5)cc4c23)cc1 ; CC(C)(C)c1ccc(N(c2ccc(-c3cccc4ccccc34)cc2)c2ccc3sc4cccc(-c5ccccc5C5CC6CCC5C6)c4c3c2)cc1]

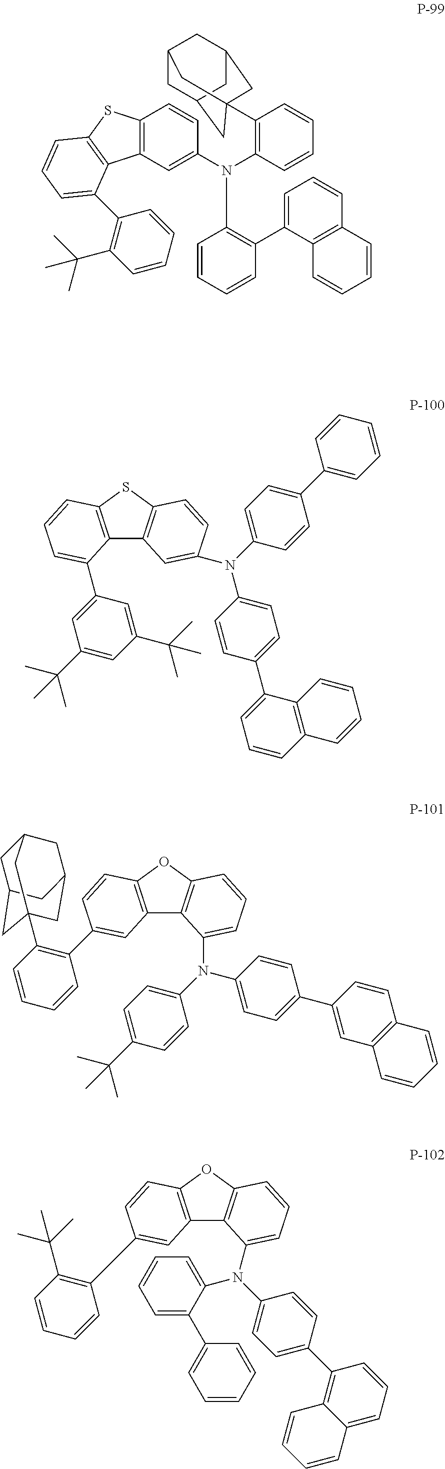 [SMILES: CC(C)(C)c1ccccc1-c1cccc2sc3ccc(N(c4ccccc4-c4cccc5ccccc45)c4ccccc4C45CC6CC(CC(C6)C4)C5)cc3c12 ; CC(C)(C)c1cc(-c2cccc3sc4ccc(N(c5ccc(-c6ccccc6)cc5)c5ccc(-c6cccc7ccccc67)cc5)cc4c23)cc(C(C)(C)C)c1 ; CC(C)(C)c1ccc(N(c2ccc(-c3ccc4ccccc4c3)cc2)c2cccc3oc4ccc(-c5ccccc5C56CC7CC(CC(C7)C5)C6)cc4c23)cc1 ; CC(C)(C)c1ccccc1-c1ccc2oc3cccc(N(c4ccc(-c5cccc6ccccc56)cc4)c4ccccc4-c4ccccc4)c3c2c1]

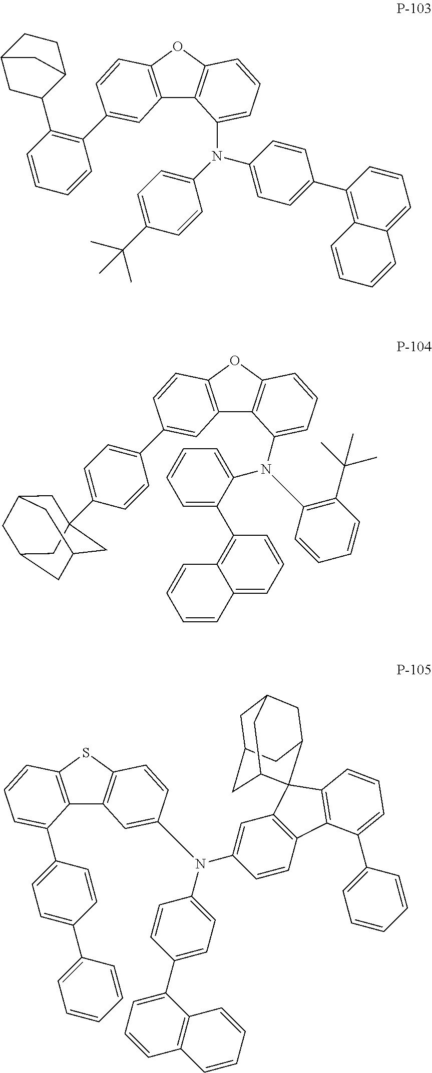 [SMILES: CC(C)(C)c1ccc(N(c2ccc(-c3cccc4ccccc34)cc2)c2cccc3oc4ccc(-c5ccccc5C5CC6CCC5C6)cc4c23)cc1 ; CC(C)(C)c1ccccc1N(c1ccccc1-c1cccc2ccccc12)c1cccc2oc3ccc(-c4ccc(C56CC7CC(CC(C7)C5)C6)cc4)cc3c12 ; c1ccc(-c2ccc(-c3cccc4sc5ccc(N(c6ccc(-c7cccc8ccccc78)cc6)c6ccc7c(c6)C6(c8cccc(-c9ccccc9)c8-7)C7CC8CC(C7)CC6C8)cc5c34)cc2)cc1]

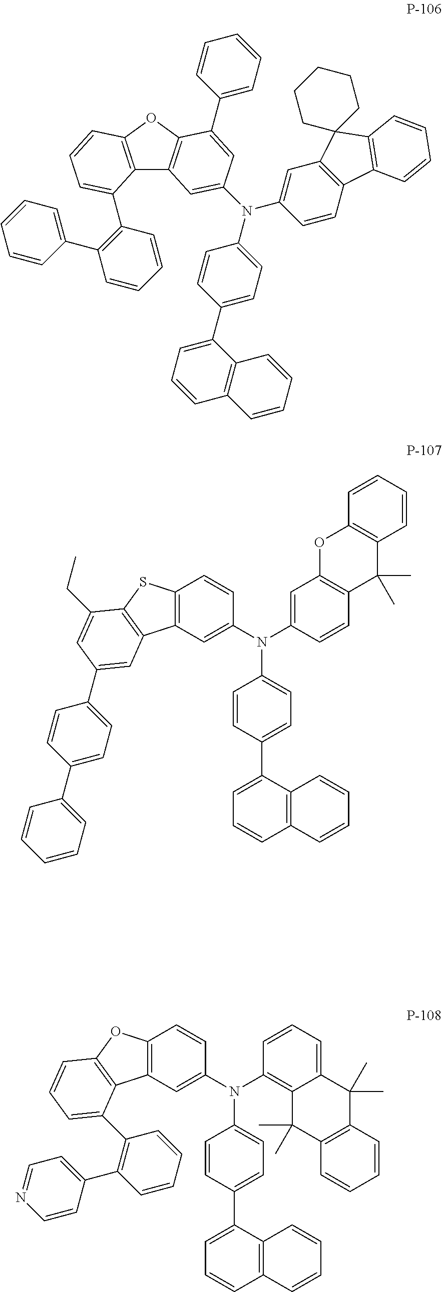 [SMILES: c1ccc(-c2ccccc2-c2cccc3oc4c(-c5ccccc5)cc(N(c5ccc(-c6cccc7ccccc67)cc5)c5ccc6c(c5)C5(CCCCC5)c5ccccc5-6)cc4c23)cc1 ; CCc1cc(-c2ccc(-c3ccccc3)cc2)cc2c1sc1ccc(N(c3ccc(-c4cccc5ccccc45)cc3)c3ccc4c(c3)Oc3ccccc3C4(C)C)cc12 ; CC1(C)c2ccccc2C(C)(C)c2c(N(c3ccc(-c4cccc5ccccc45)cc3)c3ccc4oc5cccc(-c6ccccc6-c6ccncc6)c5c4c3)cccc21]

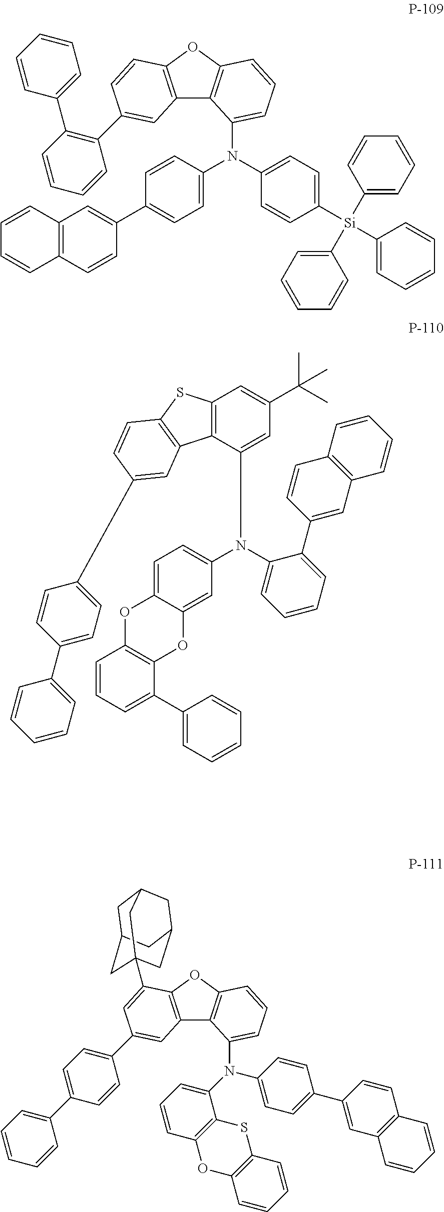

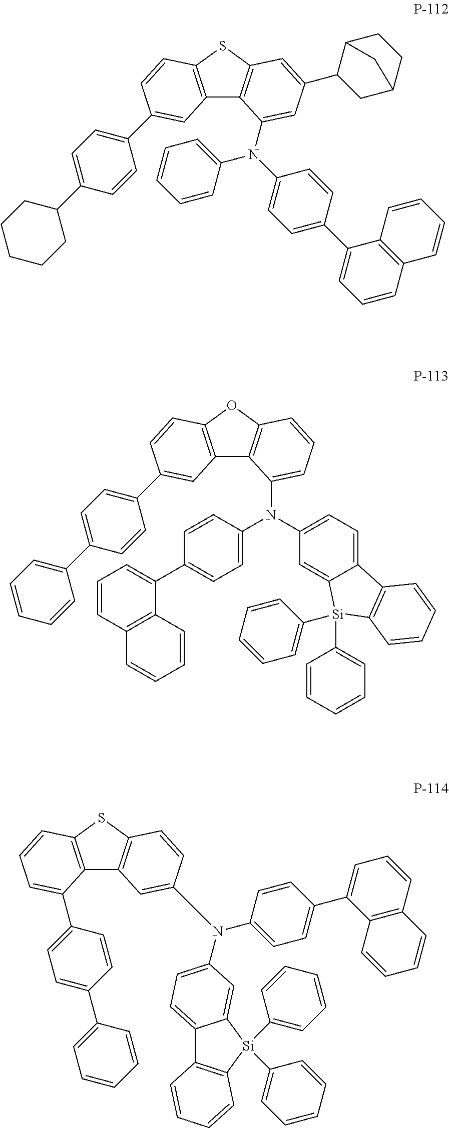 [SMILES: c1ccc(N(c2ccc(-c3cccc4ccccc34)cc2)c2cc(C3CC4CCC3C4)cc3sc4ccc(-c5ccc(C6CCCCC6)cc5)cc4c23)cc1 ; c1ccc(-c2ccc(-c3ccc4oc5cccc(N(c6ccc(-c7cccc8ccccc78)cc6)c6ccc7c(c6)[Si](c6ccccc6)(c6ccccc6)c6ccccc6-7)c5c4c3)cc2)cc1 ; c1ccc(-c2ccc(-c3cccc4sc5ccc(N(c6ccc(-c7cccc8ccccc78)cc6)c6ccc7c(c6)[Si](c6ccccc6)(c6ccccc6)c6ccccc6-7)cc5c34)cc2)cc1]

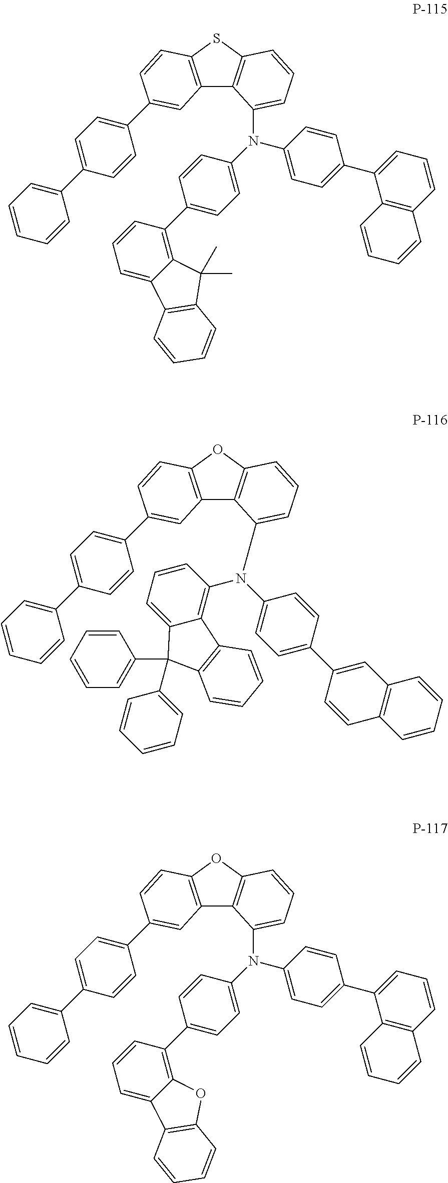 [SMILES: CC1(C)c2ccccc2-c2cccc(-c3ccc(N(c4ccc(-c5cccc6ccccc56)cc4)c4cccc5sc6ccc(-c7ccc(-c8ccccc8)cc7)cc6c45)cc3)c21 ; c1ccc(-c2ccc(-c3ccc4oc5cccc(N(c6ccc(-c7ccc8ccccc8c7)cc6)c6cccc7c6-c6ccccc6C7(c6ccccc6)c6ccccc6)c5c4c3)cc2)cc1 ; c1ccc(-c2ccc(-c3ccc4oc5cccc(N(c6ccc(-c7cccc8ccccc78)cc6)c6ccc(-c7cccc8c7oc7ccccc78)cc6)c5c4c3)cc2)cc1]

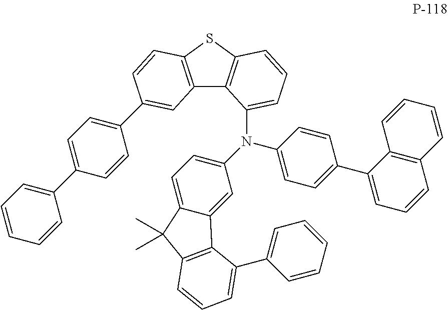 [SMILES: CC1(C)c2ccc(N(c3ccc(-c4cccc5ccccc45)cc3)c3cccc4sc5ccc(-c6ccc(-c7ccccc7)cc6)cc5c34)cc2-c2c(-c3ccccc3)cccc21]

In another aspect of the present invention, the present invention provides an organic electric element comprising a first electrode, a second electrode, and an organic material layer between the first electrode and the second electrode, wherein the organic material layer comprises compound represented by the Formula 1.

The organic material layer includes a light-emitting layer and a hole transport zone between the first electrode and the light-emitting layer, and the hole transport zone includes the compound represented by Formula 1. Here, the hole transport zone includes an emission auxiliary layer, and the compound of Formula 1 may be included in the emission auxiliary layer.

In addition, the organic material layer includes a light-emitting layer and a hole transport layer between the first electrode and the light-emitting layer, and an emission auxiliary layer between the hole transport layer and the first electrode, the compound is included in at least one layer of the hole transport layer and the emission auxiliary layer, preferably, in the emission auxiliary layer.

The organic electric element may further include a layer for improving luminous efficiency. Here, the layer for improving luminous efficiency is formed on one side of two sides of a first electrode or a second electrode, the one side is not facing the organic material layer. In addition, the compound represented by Formula 1 may be included in the light efficiency improvement layer.

The organic material layer may comprise two or more stacks, wherein the stacks may comprise a hole transport layer, a light emitting layer, and an electron transport layer formed in sequence on the anode, and a charge generation layer may be formed between the two or more stacks.

In another aspect of the present invention, the present invention provides an electronic device comprising a display device and a control unit for controlling the display device, wherein the display device comprises the compound represented by Formula 1.

Hereinafter, a synthesis example of the compound represented by Formula 1 and a preparation method of an organic electroluminescent element will be described in detail byway of examples. However, the present invention is not limited to the following examples.

### Synthesis Example

The compound (final product) represented by Formula 1 of the present invention can be synthesized as shown in Reaction Scheme 1 below, but is not limited thereto.

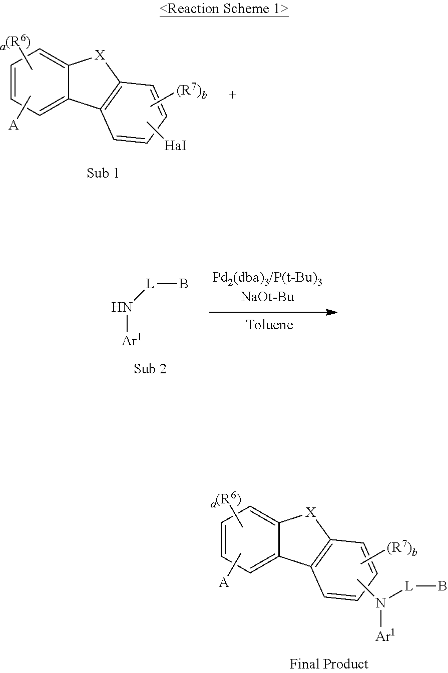

### Synthesis Example of Sub1

Sub1 of Reaction Scheme 1 may be synthesized through the reaction route of Reaction Scheme 2 below, but is not limited thereto.

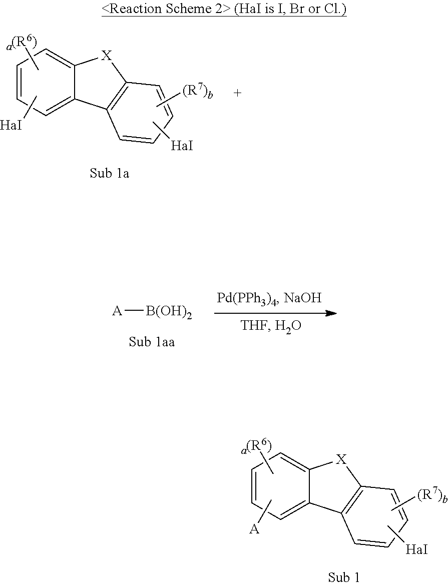

### 1. Synthesis Example of Sub 1-13

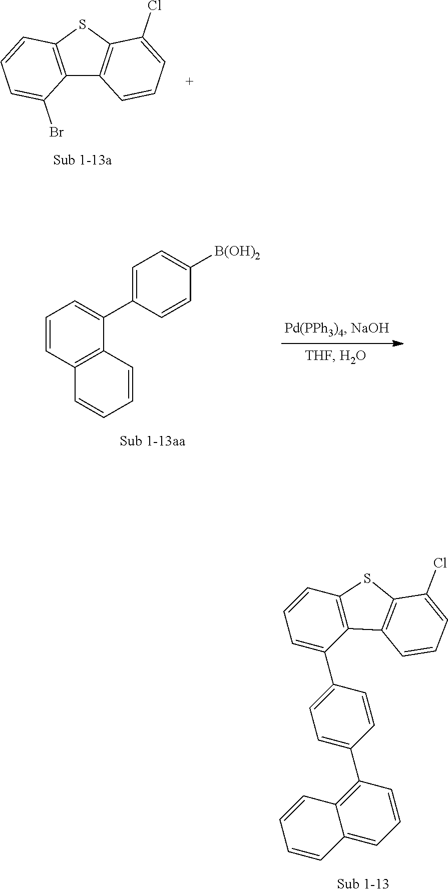 [SMILES: Clc1cccc2c1sc1cccc(Br)c12 ; OB(O)c1ccc(-c2cccc3ccccc23)cc1 ; Clc1cccc2c1sc1cccc(-c3ccc(-c4cccc5ccccc45)cc3)c12]

After dissolving Sub 1-13a (50.0 g, 168.0 mmol) in THF (840 ml), Sub 1-13aa (41.7 g, 168.0 mmol), Pd(PPh₃)₄ (11.7 g, 10.1 mmol), NaOH (20.2 g, 504.0 mmol) and water (420 ml) were added to the solution and the reaction was carried out at 80° C. When the reaction was completed, the reaction product was extracted with CH₂Cl₂ and water, and then an organic layer was dried with MgSO₄ and concentrated. Then, the concentrate was separated through a silica gel column and recrystallized to obtain 57.4 g (yield: 81.1%) of the product.

### 2. Synthesis Example of Sub 1-21

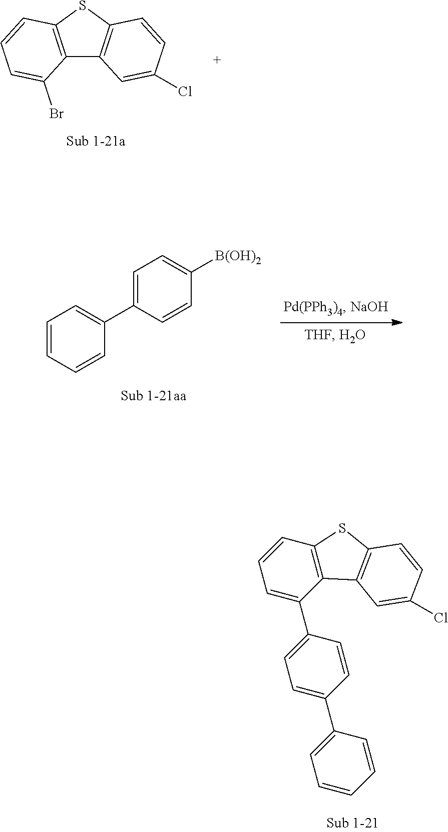 [SMILES: Clc1ccc2sc3cccc(Br)c3c2c1 ; OB(O)c1ccc(-c2ccccc2)cc1 ; Clc1ccc2sc3cccc(-c4ccc(-c5ccccc5)cc4)c3c2c1]

After dissolving Sub 1-21a (50.0 g, 168.0 mmol) in THE (840 ml), Sub 1-21aa (33.3 g, 168.0 mmol), Pd(PPh₃)₄ (11.7 g, 10.1 mmol), NaOH (20.2 g, 504.0 mmol) and water (420 ml) were added to the solution, and then the reaction was carried out in the same manner as the synthesis method of Sub 1-13, and then purified to obtain 50.1 g of product. (yield: 80.4%)

### 3. Synthesis Example of Sub 1-45

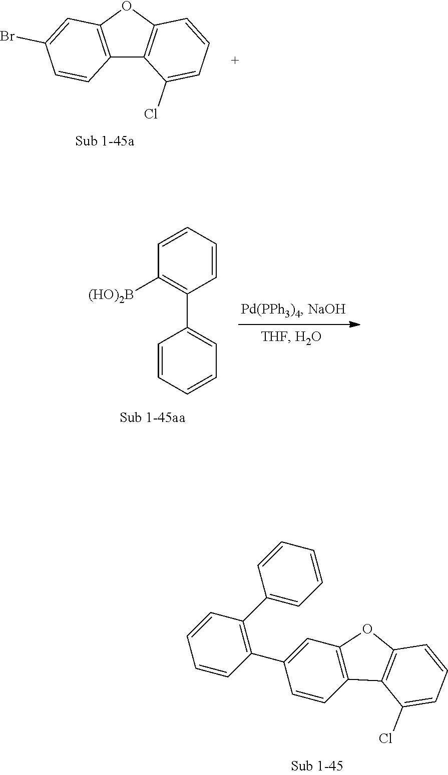 [SMILES: Clc1cccc2oc3cc(Br)ccc3c12 ; OB(O)c1ccccc1-c1ccccc1 ; Clc1cccc2oc3cc(-c4ccccc4-c4ccccc4)ccc3c12]

After dissolving Sub 1-45a (50.0 g, 177.6 mmol) in THE (888 ml), Sub 1-45aa (35.2 g, 177.6 mmol), Pd(PPh₃)₄ (12.3 g, 10.7 mmol), NaOH (21.3 g, 532.8 mmol) and water (444 ml) were added to the solution, and then the reaction was carried out in the same manner as the synthesis method of Sub 1-13, and then purified to obtain 51.9 g (yield: 82.3%) of product.

### 4. Synthesis Example of Sub 1-71

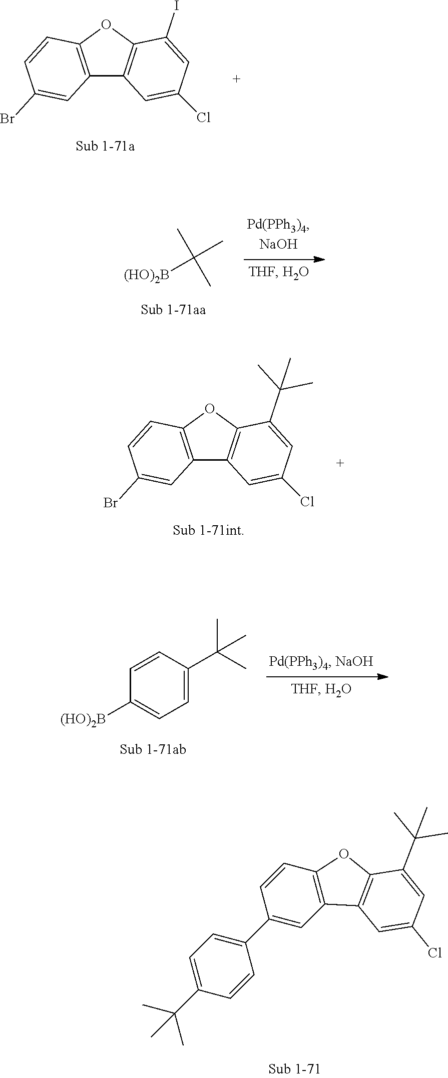

### Synthesis Example of Sub 1-71Int

After dissolving Sub 1-71a (50.0 g, 122.7 mmol) in THE (614 ml), Sub 1-71aa (12.5 g, 122.7 mmol), Pd(PPh₃)₄ (8.5 g, 7.4 mmol), NaOH (14.7 g, 368.2 mmol) and water (307 ml) were added to the solution, and then the reaction was carried out in the same manner as the synthesis method of Sub 1-13, and then purified to obtain 35.4 g (yield: 85.5%) of product.

### Synthesis Example of Sub 1-71

After dissolving Sub 1-71int. (35.4 g, 104.8 mmol) in THE (524 ml), Sub 1-71ab (18.7 g, 104.8 mmol), Pd(PPh₃)₄ (7.3 g, 6.3 mmol), NaOH (12.6 g, 314.5 mmol) and water (262 ml) were added to the solution, and then the reaction was carried out in the same manner as the synthesis method of Sub 1-13, and then purified to obtain 32.7 g (yield: 79.8%) of product.

### 5. Synthesis Example of Sub 1-83

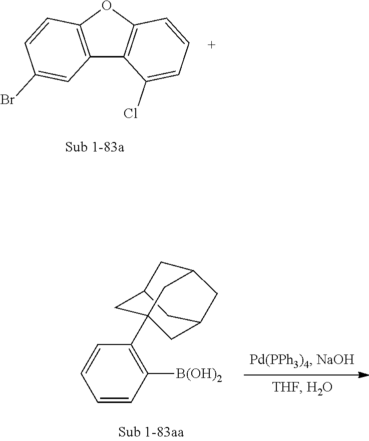 [SMILES: Clc1cccc2oc3ccc(Br)cc3c12 ; OB(O)c1ccccc1C12CC3CC(CC(C3)C1)C2]

After dissolving Sub 1-83a (50.0 g, 177.6 mmol) in THE (888 ml), Sub 1-83aa (45.5 g, 177.6 mmol), Pd(PPh₃)₄ (12.3 g, 10.7 mmol), NaOH (21.3 g, 532.8 mmol) and water (444 ml) were added to the solution, and then the reaction was carried out in the same manner as the synthesis method of Sub 1-13, and then purified to obtain 60.7 g (yield: 82.7%) of product.

Compounds belonging to Sub 1 may be, but not limited to, the following compounds, and Table 1 shows FD-MS (Field Desorption-Mass Spectrometry) values of the following compounds.

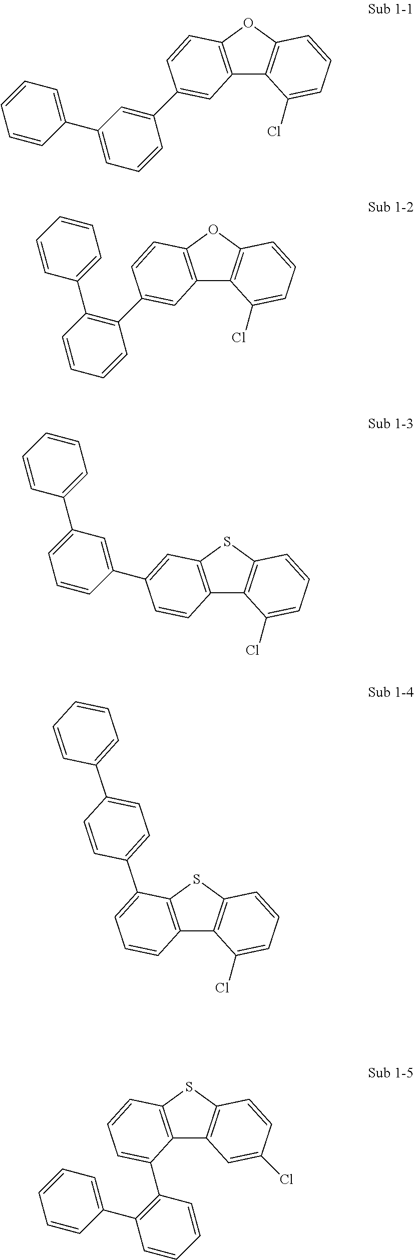 [SMILES: Clc1cccc2oc3ccc(-c4cccc(-c5ccccc5)c4)cc3c12 ; Clc1cccc2oc3ccc(-c4ccccc4-c4ccccc4)cc3c12 ; Clc1cccc2sc3cc(-c4cccc(-c5ccccc5)c4)ccc3c12 ; Clc1cccc2sc3c(-c4ccc(-c5ccccc5)cc4)cccc3c12 ; Clc1ccc2sc3cccc(-c4ccccc4-c4ccccc4)c3c2c1]

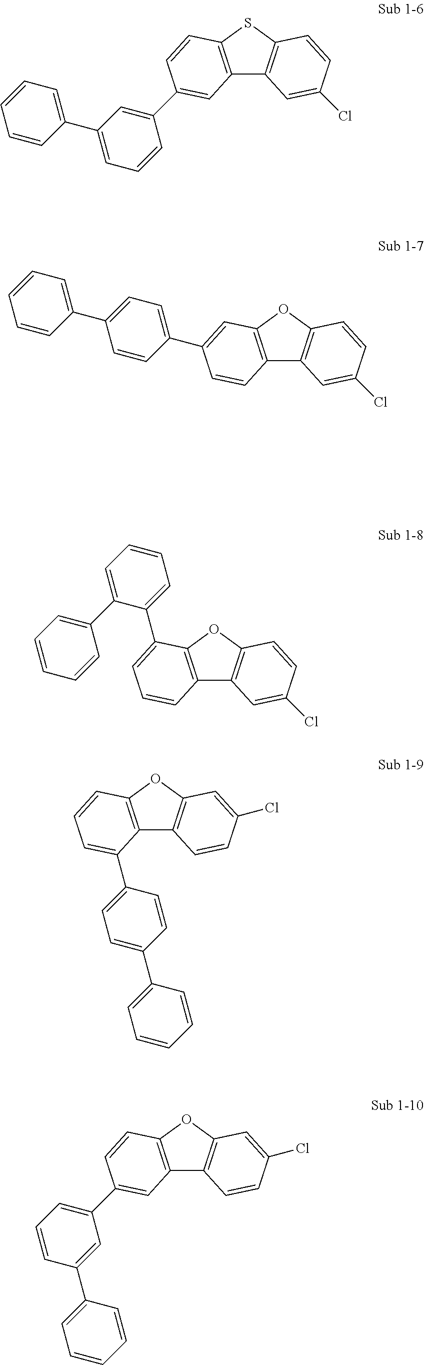 [SMILES: Clc1ccc2sc3ccc(-c4cccc(-c5ccccc5)c4)cc3c2c1 ; Clc1ccc2oc3cc(-c4ccc(-c5ccccc5)cc4)ccc3c2c1 ; Clc1ccc2oc3c(-c4ccccc4-c4ccccc4)cccc3c2c1 ; Clc1ccc2c(c1)oc1cccc(-c3ccc(-c4ccccc4)cc3)c12 ; Clc1ccc2c(c1)oc1ccc(-c3cccc(-c4ccccc4)c3)cc12]

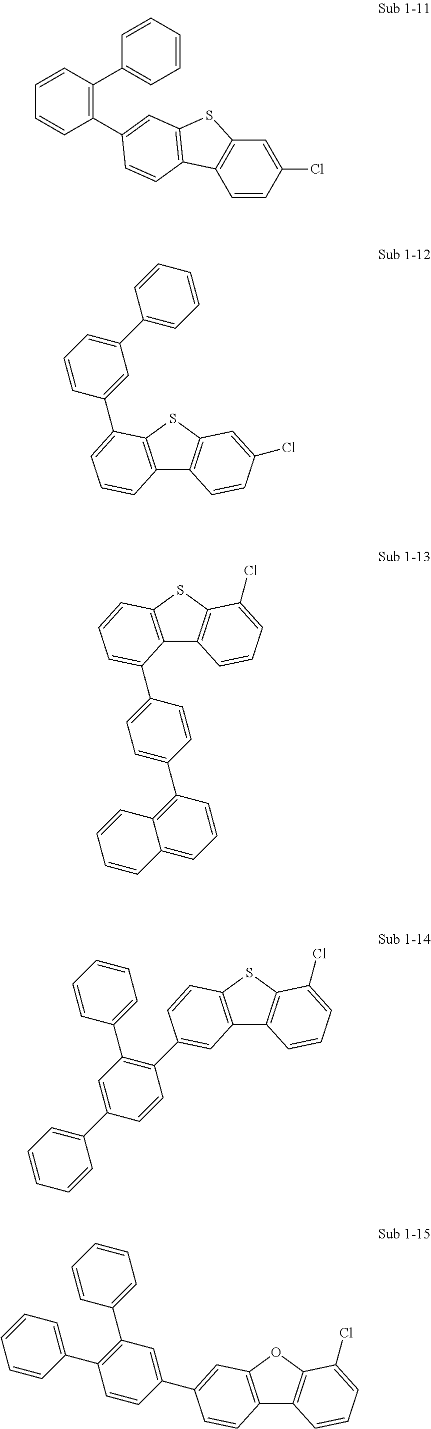 [SMILES: Clc1ccc2c(c1)sc1cc(-c3ccccc3-c3ccccc3)ccc12 ; Clc1ccc2c(c1)sc1c(-c3cccc(-c4ccccc4)c3)cccc12 ; Clc1cccc2c1sc1cccc(-c3ccc(-c4cccc5ccccc45)cc3)c12 ; Clc1cccc2c1sc1ccc(-c3ccc(-c4ccccc4)cc3-c3ccccc3)cc12 ; Clc1cccc2c1oc1cc(-c3ccc(-c4ccccc4)c(-c4ccccc4)c3)ccc12]

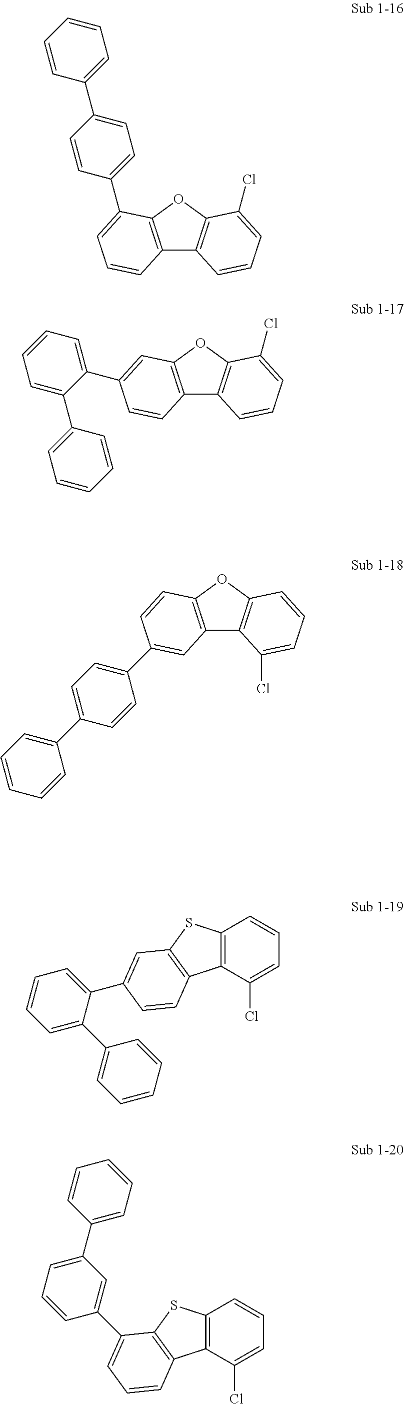 [SMILES: Clc1cccc2c1oc1c(-c3ccc(-c4ccccc4)cc3)cccc12 ; Clc1cccc2c1oc1cc(-c3ccccc3-c3ccccc3)ccc12 ; Clc1cccc2oc3ccc(-c4ccc(-c5ccccc5)cc4)cc3c12 ; Clc1cccc2sc3cc(-c4ccccc4-c4ccccc4)ccc3c12 ; Clc1cccc2sc3c(-c4cccc(-c5ccccc5)c4)cccc3c12]

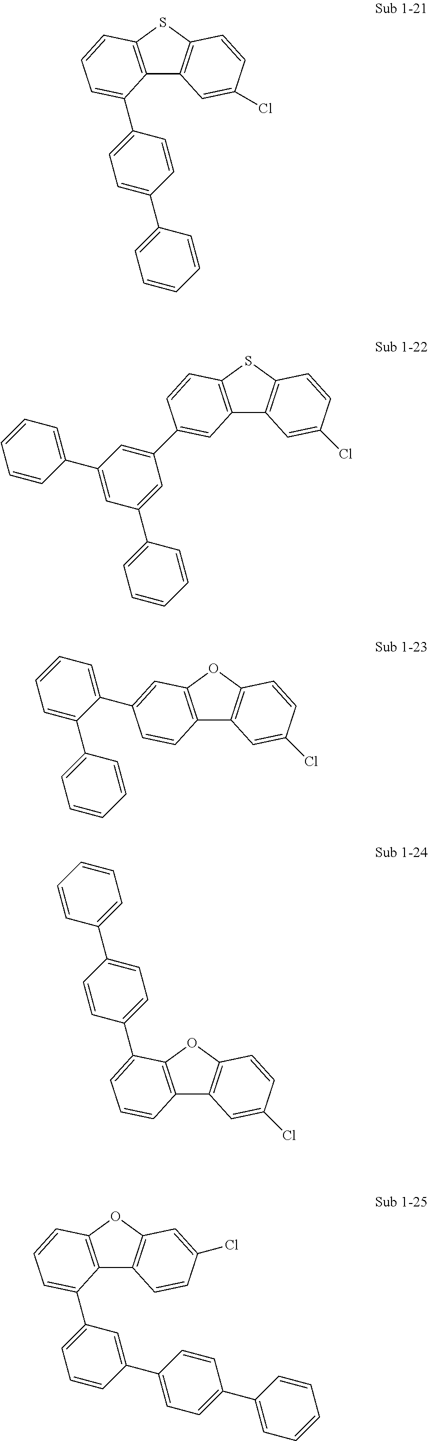 [SMILES: Clc1ccc2sc3cccc(-c4ccc(-c5ccccc5)cc4)c3c2c1 ; Clc1ccc2sc3ccc(-c4cc(-c5ccccc5)cc(-c5ccccc5)c4)cc3c2c1 ; Clc1ccc2oc3cc(-c4ccccc4-c4ccccc4)ccc3c2c1 ; Clc1ccc2oc3c(-c4ccc(-c5ccccc5)cc4)cccc3c2c1 ; Clc1ccc2c(c1)oc1cccc(-c3cccc(-c4ccc(-c5ccccc5)cc4)c3)c12]

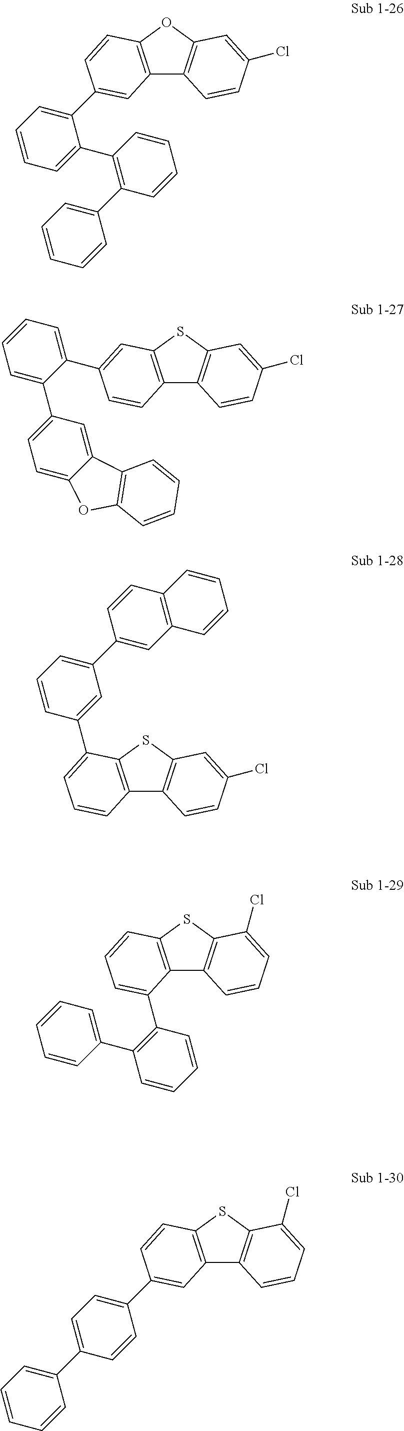 [SMILES: Clc1ccc2c(c1)oc1ccc(-c3ccccc3-c3ccccc3-c3ccccc3)cc12 ; Clc1ccc2c(c1)sc1cc(-c3ccccc3-c3ccc4oc5ccccc5c4c3)ccc12 ; Clc1ccc2c(c1)sc1c(-c3cccc(-c4ccc5ccccc5c4)c3)cccc12 ; Clc1cccc2c1sc1cccc(-c3ccccc3-c3ccccc3)c12 ; Clc1cccc2c1sc1ccc(-c3ccc(-c4ccccc4)cc3)cc12]

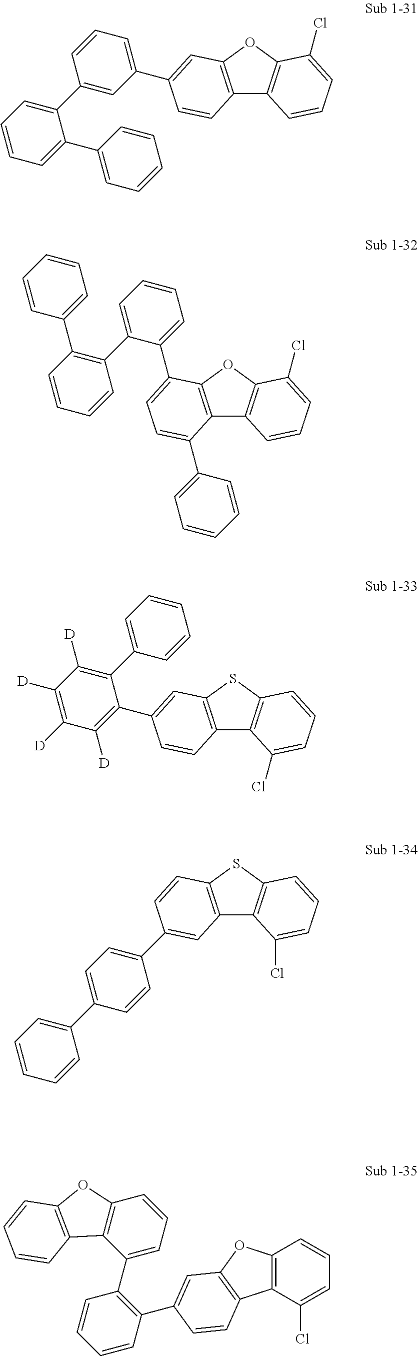 [SMILES: Clc1cccc2c1oc1cc(-c3cccc(-c4ccccc4-c4ccccc4)c3)ccc12 ; Clc1cccc2c1oc1c(-c3ccccc3-c3ccccc3-c3ccccc3)ccc(-c3ccccc3)c12 ; [2H]c1c([2H])c([2H])c(-c2ccc3c(c2)sc2cccc(Cl)c23)c(-c2ccccc2)c1[2H] ; Clc1cccc2sc3ccc(-c4ccc(-c5ccccc5)cc4)cc3c12 ; Clc1cccc2oc3cc(-c4ccccc4-c4cccc5oc6ccccc6c45)ccc3c12]

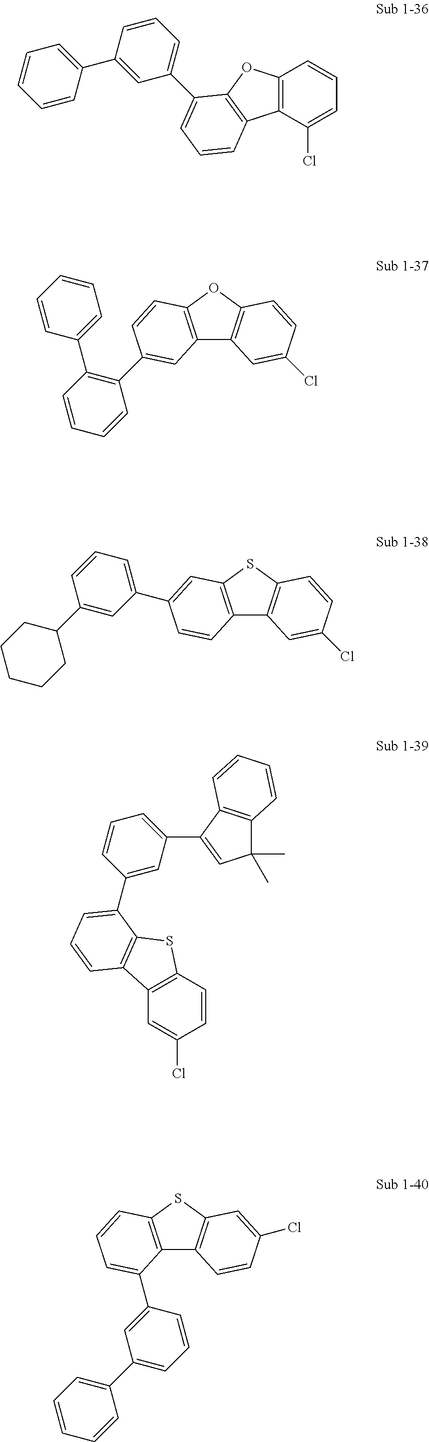 [SMILES: Clc1cccc2oc3c(-c4cccc(-c5ccccc5)c4)cccc3c12 ; Clc1ccc2oc3ccc(-c4ccccc4-c4ccccc4)cc3c2c1 ; Clc1ccc2sc3cc(-c4cccc(C5CCCCC5)c4)ccc3c2c1 ; CC1(C)C=C(c2cccc(-c3cccc4c3sc3ccc(Cl)cc34)c2)c2ccccc21 ; Clc1ccc2c(c1)sc1cccc(-c3cccc(-c4ccccc4)c3)c12]

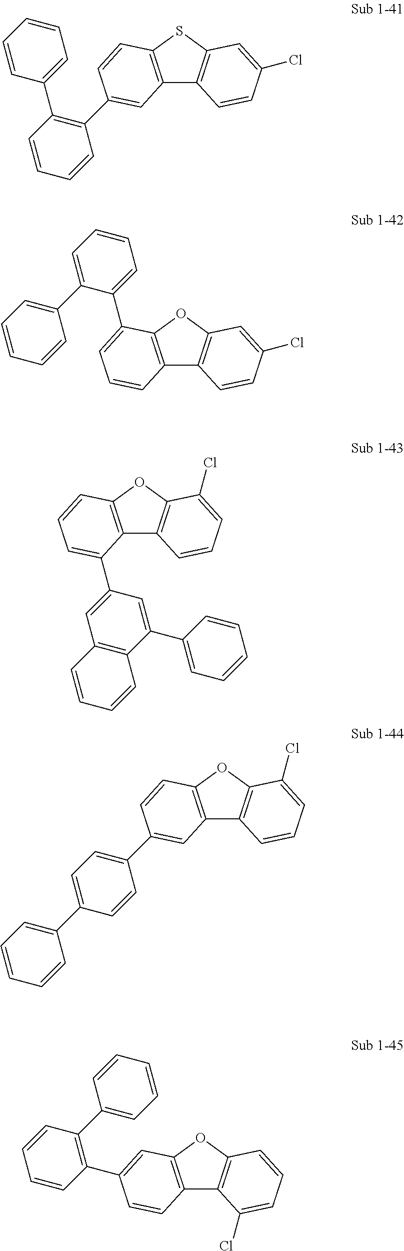 [SMILES: Clc1ccc2c(c1)sc1ccc(-c3ccccc3-c3ccccc3)cc12 ; Clc1ccc2c(c1)oc1c(-c3ccccc3-c3ccccc3)cccc12 ; Clc1cccc2c1oc1cccc(-c3cc(-c4ccccc4)c4ccccc4c3)c12 ; Clc1cccc2c1oc1ccc(-c3ccc(-c4ccccc4)cc3)cc12 ; Clc1cccc2oc3cc(-c4ccccc4-c4ccccc4)ccc3c12]

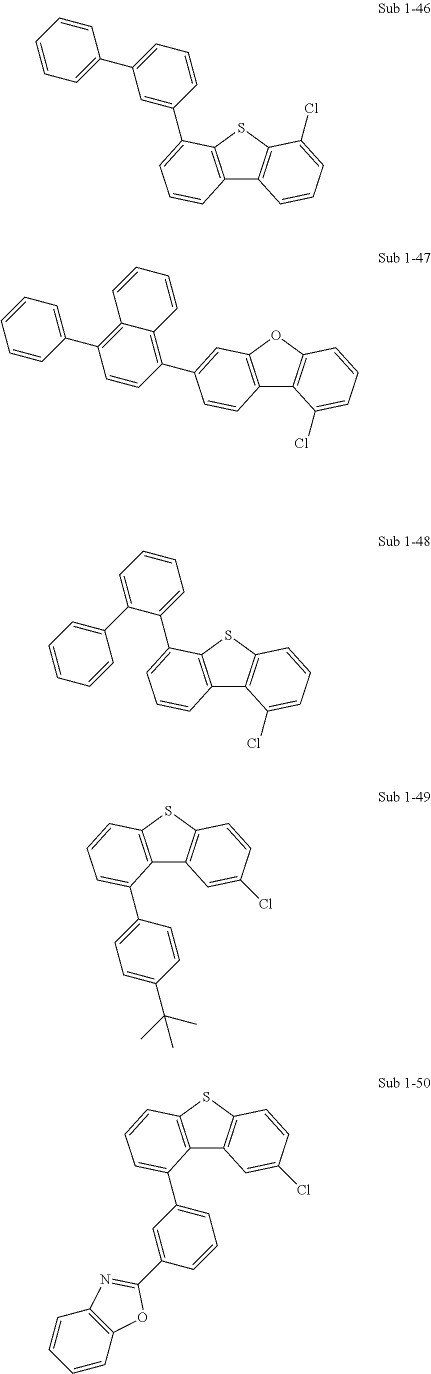 [SMILES: Clc1cccc2c1sc1c(-c3cccc(-c4ccccc4)c3)cccc12 ; Clc1cccc2oc3cc(-c4ccc(-c5ccccc5)c5ccccc45)ccc3c12 ; Clc1cccc2sc3c(-c4ccccc4-c4ccccc4)cccc3c12 ; CC(C)(C)c1ccc(-c2cccc3sc4ccc(Cl)cc4c23)cc1 ; Clc1ccc2sc3cccc(-c4cccc(-c5nc6ccccc6o5)c4)c3c2c1]

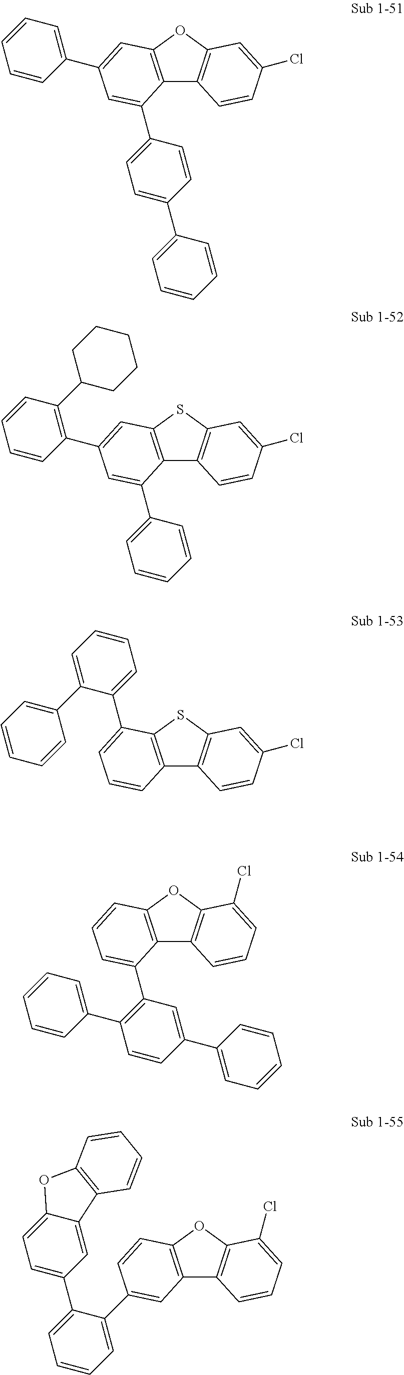 [SMILES: Clc1ccc2c(c1)oc1cc(-c3ccccc3)cc(-c3ccc(-c4ccccc4)cc3)c12 ; Clc1ccc2c(c1)sc1cc(-c3ccccc3C3CCCCC3)cc(-c3ccccc3)c12 ; Clc1ccc2c(c1)sc1c(-c3ccccc3-c3ccccc3)cccc12 ; Clc1cccc2c1oc1cccc(-c3cc(-c4ccccc4)ccc3-c3ccccc3)c12 ; Clc1cccc2c1oc1ccc(-c3ccccc3-c3ccc4oc5ccccc5c4c3)cc12]

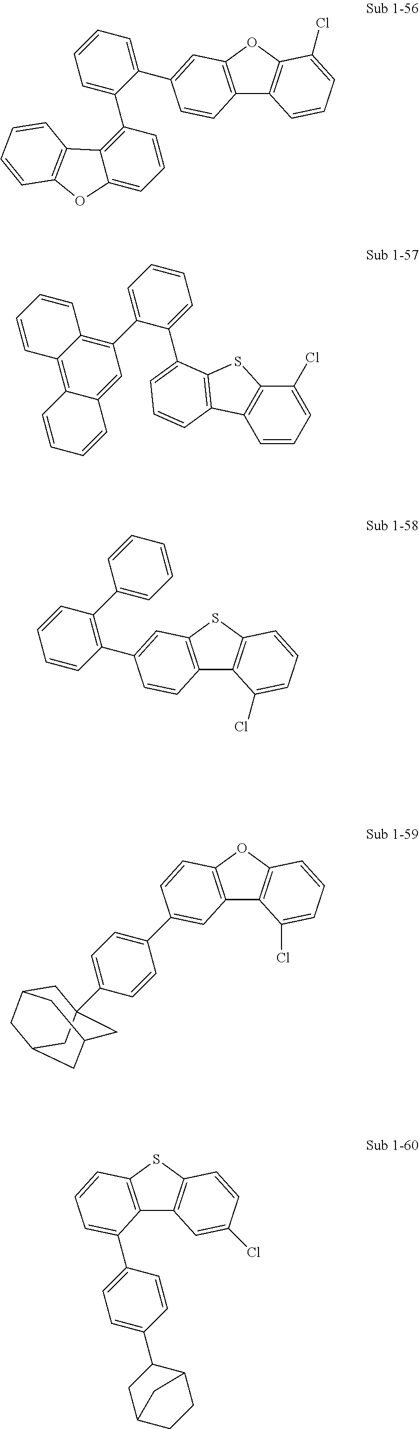 [SMILES: Clc1cccc2c1oc1cc(-c3ccccc3-c3cccc4oc5ccccc5c34)ccc12 ; Clc1cccc2c1sc1c(-c3ccccc3-c3cc4ccccc4c4ccccc34)cccc12 ; Clc1cccc2sc3cc(-c4ccccc4-c4ccccc4)ccc3c12 ; Clc1cccc2oc3ccc(-c4ccc(C56CC7CC(CC(C7)C5)C6)cc4)cc3c12 ; Clc1ccc2sc3cccc(-c4ccc(C5CC6CCC5C6)cc4)c3c2c1]

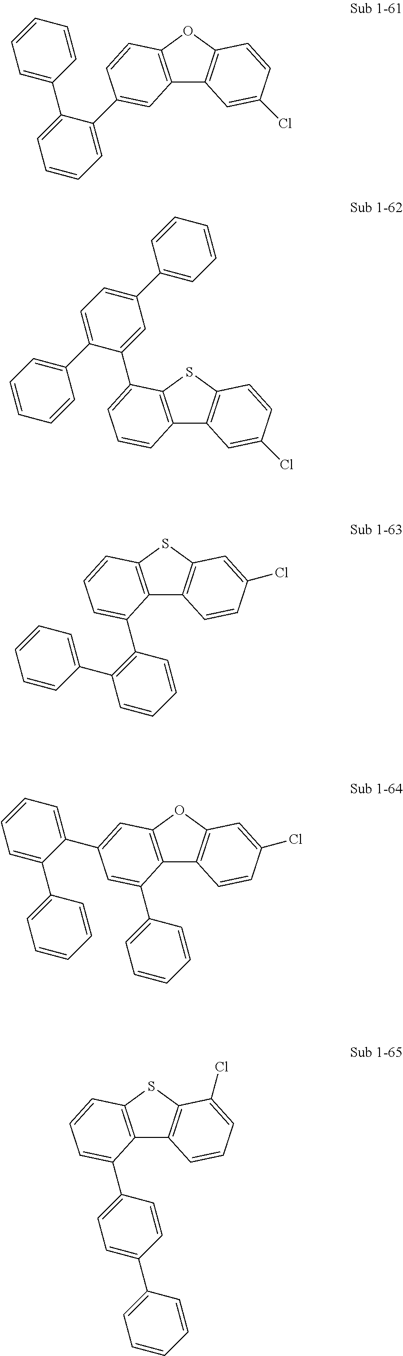 [SMILES: Clc1ccc2oc3ccc(-c4ccccc4-c4ccccc4)cc3c2c1 ; Clc1ccc2sc3c(-c4cc(-c5ccccc5)ccc4-c4ccccc4)cccc3c2c1 ; Clc1ccc2c(c1)sc1cccc(-c3ccccc3-c3ccccc3)c12 ; Clc1ccc2c(c1)oc1cc(-c3ccccc3-c3ccccc3)cc(-c3ccccc3)c12 ; Clc1cccc2c1sc1cccc(-c3ccc(-c4ccccc4)cc3)c12]

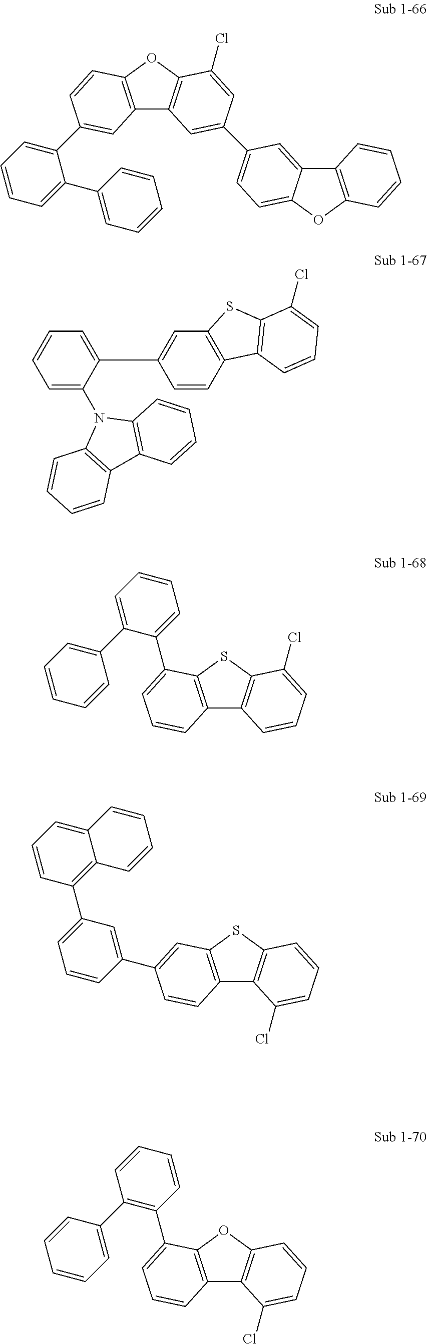 [SMILES: Clc1cc(-c2ccc3oc4ccccc4c3c2)cc2c1oc1ccc(-c3ccccc3-c3ccccc3)cc12 ; Clc1cccc2c1sc1cc(-c3ccccc3-n3c4ccccc4c4ccccc43)ccc12 ; Clc1cccc2c1sc1c(-c3ccccc3-c3ccccc3)cccc12 ; Clc1cccc2sc3cc(-c4cccc(-c5cccc6ccccc56)c4)ccc3c12 ; Clc1cccc2oc3c(-c4ccccc4-c4ccccc4)cccc3c12]

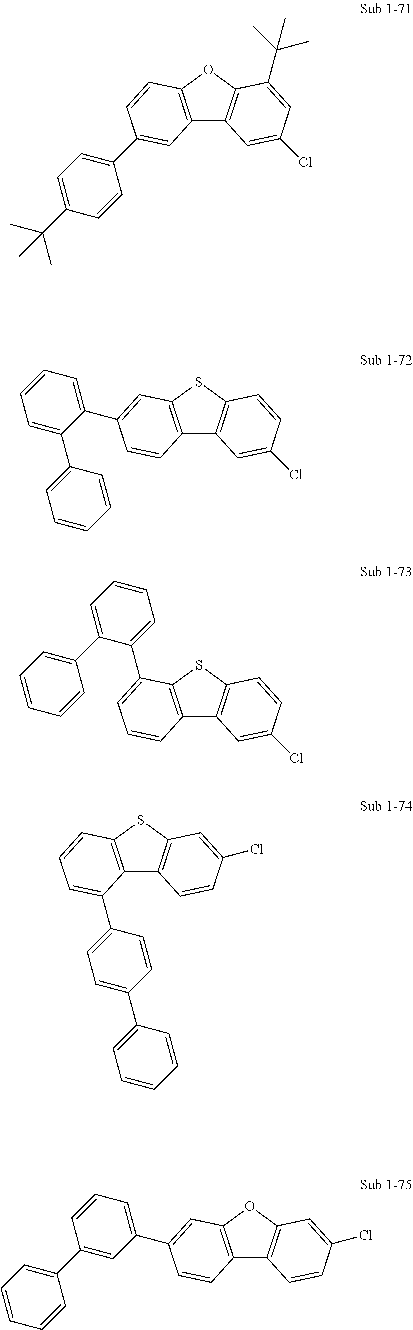 [SMILES: CC(C)(C)c1ccc(-c2ccc3oc4c(C(C)(C)C)cc(Cl)cc4c3c2)cc1 ; Clc1ccc2sc3cc(-c4ccccc4-c4ccccc4)ccc3c2c1 ; Clc1ccc2sc3c(-c4ccccc4-c4ccccc4)cccc3c2c1 ; Clc1ccc2c(c1)sc1cccc(-c3ccc(-c4ccccc4)cc3)c12 ; Clc1ccc2c(c1)oc1cc(-c3cccc(-c4ccccc4)c3)ccc12]

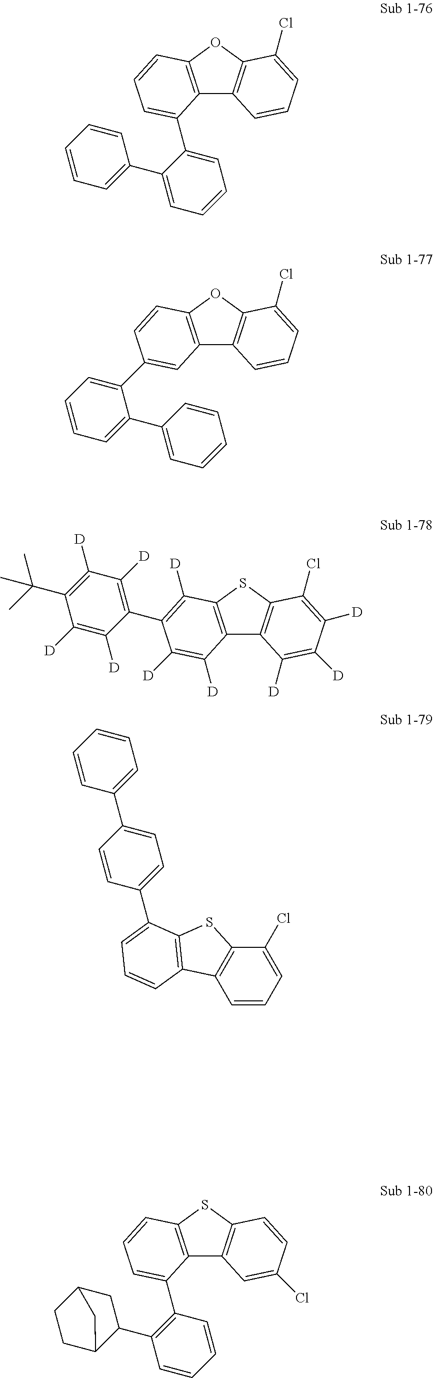 [SMILES: Clc1cccc2c1oc1cccc(-c3ccccc3-c3ccccc3)c12 ; Clc1cccc2c1oc1ccc(-c3ccccc3-c3ccccc3)cc12 ; [2H]c1c([2H])c(C(C)(C)C)c([2H])c([2H])c1-c1c([2H])c([2H])c2c(sc3c(Cl)c([2H])c([2H])c([2H])c32)c1[2H] ; Clc1cccc2c1sc1c(-c3ccc(-c4ccccc4)cc3)cccc12 ; Clc1ccc2sc3cccc(-c4ccccc4C4CC5CCC4C5)c3c2c1]

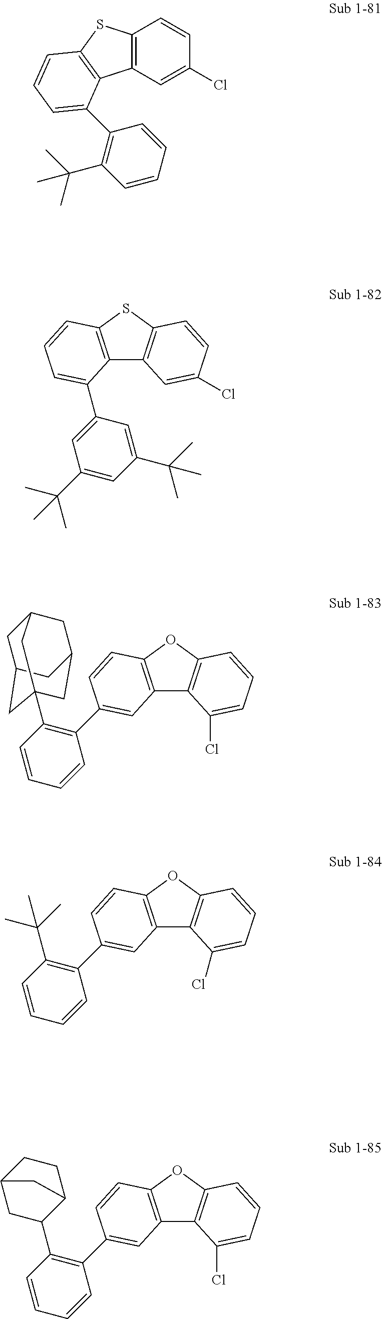 [SMILES: CC(C)(C)c1ccccc1-c1cccc2sc3ccc(Cl)cc3c12 ; CC(C)(C)c1cc(-c2cccc3sc4ccc(Cl)cc4c23)cc(C(C)(C)C)c1 ; Clc1cccc2oc3ccc(-c4ccccc4C45CC6CC(CC(C6)C4)C5)cc3c12 ; CC(C)(C)c1ccccc1-c1ccc2oc3cccc(Cl)c3c2c1 ; Clc1cccc2oc3ccc(-c4ccccc4C4CC5CCC4C5)cc3c12]

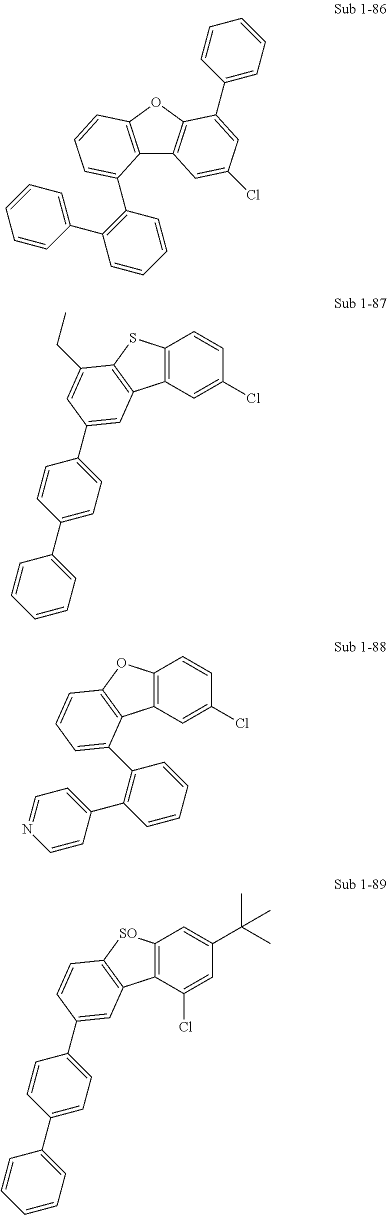

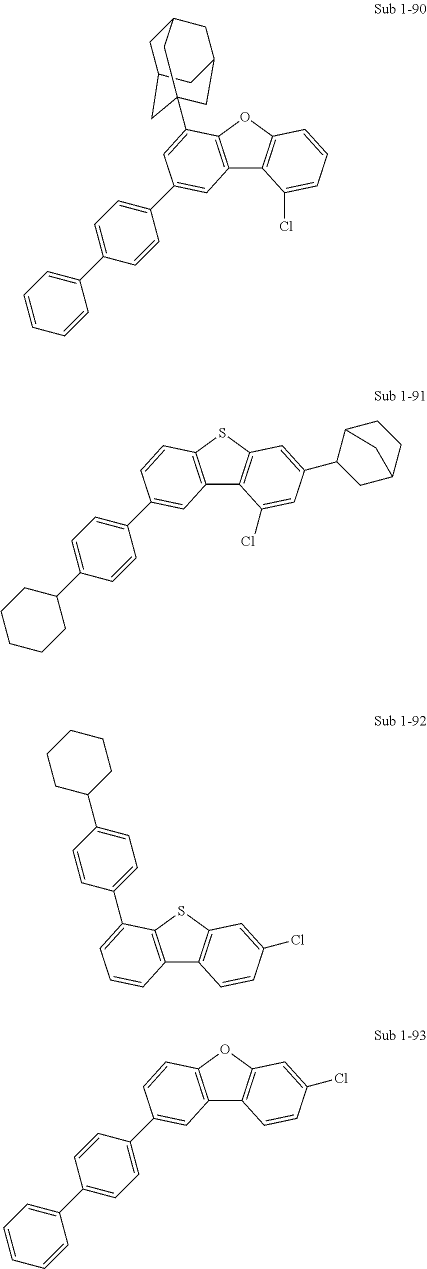 [SMILES: Clc1cccc2oc3c(C45CC6CC(CC(C6)C4)C5)cc(-c4ccc(-c5ccccc5)cc4)cc3c12 ; Clc1cc(C2CC3CCC2C3)cc2sc3ccc(-c4ccc(C5CCCCC5)cc4)cc3c12 ; Clc1ccc2c(c1)sc1c(-c3ccc(C4CCCCC4)cc3)cccc12 ; Clc1ccc2c(c1)oc1ccc(-c3ccc(-c4ccccc4)cc3)cc12]

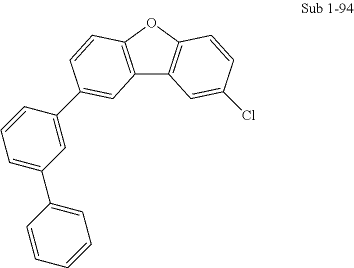 [SMILES: Clc1ccc2oc3ccc(-c4cccc(-c5ccccc5)c4)cc3c2c1]

TABLE 1

| Compound | FD-MS | Compound | FD-MS |
|---|---|---|---|
| Sub 1-1 | m/z = 354.08(C₂₄H₁₅ClO = 354.83) | Sub 1-2 | m/z = 354.08(C₂₄H₁₅ClO = 354.83) |
| Sub 1-3 | m/z = 370.06(C₂₄H₁₅ClS = 370.89) | Sub 1-4 | m/z = 370.06(C₂₄H₁₅ClS = 370.89) |
| Sub 1-5 | m/z = 370.06(C₂₄H₁₅ClS = 370.89) | Sub 1-6 | m/z = 370.06(C₂₄H₁₅ClS = 370.89) |
| Sub 1-7 | m/z = 354.08(C₂₄H₁₅ClO = 354.83) | Sub 1-8 | m/z = 354.08(C₂₄H₁₅ClO = 354.83) |
| Sub 1-9 | m/z = 354.08(C₂₄H₁₅ClO = 354.83) | Sub 1-10 | m/z = 354.08(C₂₄H₁₅ClO = 354.83) |
| Sub 1-11 | m/z = 370.06(C₂₄H₁₅ClS = 370.89) | Sub 1-12 | m/z = 370.06(C₂₄H₁₅ClS = 370.89) |
| Sub 1-13 | m/z = 420.07(C₂₈H₁₇ClS = 420.95) | Sub 1-14 | m/z = 446.09(C₃₀H₁₉ClS = 446.99) |
| Sub 1-15 | m/z = 430.11(C₃₀H₁₉ClO = 430.93) | Sub 1-16 | m/z = 354.08(C₂₄H₁₅ClO = 354.83) |
| Sub 1-17 | m/z = 354.08(C₂₄H₁₅ClO = 354.83) | Sub 1-18 | m/z = 354.08(C₂₄H₁₅ClO = 354.83) |
| Sub 1-19 | m/z = 370.06(C₂₄H₁₅ClS = 370.89) | Sub 1-20 | m/z = 370.06(C₂₄H₁₅ClS = 370.89) |
| Sub 1-21 | m/z = 370.06(C₂₄H₁₅ClS = 370.89) | Sub 1-22 | m/z = 446.09(C₃₀H₁₉ClS = 446.99) |
| Sub 1-23 | m/z = 354.08(C₂₄H₁₅ClO = 354.83) | Sub 1-24 | m/z = 354.08(C₂₄H₁₅ClO = 354.83) |
| Sub 1-25 | m/z = 430.11(C₃₀H₁₉ClO = 430.93) | Sub 1-26 | m/z = 430.11(C₃₀H₁₉ClO = 430.93) |
| Sub 1-27 | m/z = 460.07(C₃₀H₁₇ClOS = 460.98) | Sub 1-28 | m/z = 420.07(C₂₈H₁₇ClS = 420.95) |
| Sub 1-29 | m/z = 370.06(C₂₄H₁₅ClS = 370.89) | Sub 1-30 | m/z = 370.06(C₂₄H₁₅ClS = 370.89) |
| Sub 1-31 | m/z = 430.11(C₃₀H₁₉ClO = 430.93) | Sub 1-32 | m/z = 506.14(C₃₆H₂₃ClO = 507.03) |
| Sub 1-33 | m/z = 374.08(C₂₄H₁₁D₄ClS = 374.92) | Sub 1-34 | m/z = 370.06(C₂₄H₁₅ClS = 370.89) |
| Sub 1-35 | m/z = 444.09(C₃₀H₁₇ClO₂ = 444.91) | Sub 1-36 | m/z = 354.08(C₂₄H₁₅ClO = 354.83) |
| Sub 1-37 | m/z = 354.08(C₂₄H₁₅ClO = 354.83) | Sub 1-38 | m/z = 376.11(C₂₄H₂₁ClS = 376.94) |
| Sub 1-39 | m/z = 436.11(C₂₉H₂₁ClS = 437.00) | Sub 1-40 | m/z = 370.06(C₂₄H₁₅ClS = 370.89) |
| Sub 1-41 | m/z = 370.06(C₂₄H₁₅ClS = 370.89) | Sub 1-42 | m/z = 354.08(C₂₄H₁₅ClO = 354.83) |
| Sub 1-43 | m/z = 404.1(C₂₈H₁₇ClO = 404.89) | Sub 1-44 | m/z = 354.08(C₂₄H₁₅ClO = 354.83) |
| Sub 1-45 | m/z = 354.08(C₂₄H₁₅ClO = 354.83) | Sub 1-46 | m/z = 370.06(C₂₄H₁₅ClS = 370.89) |
| Sub 1-47 | m/z = 404.10(C₂₈H₁₇ClO = 404.89) | Sub 1-48 | m/z = 370.06(C₂₄H₁₅ClS = 370.89) |
| Sub 1-49 | m/z = 334.11(C₂₂H₁₉ClO = 334.84) | Sub 1-50 | m/z = 411.05(C₂₅H₁₄ClNOS = 411.90) |
| Sub 1-51 | m/z = 430.11(C₃₀H₁₉ClO = 430.93) | Sub 1-52 | m/z = 452.14(C₃₀H₂₅ClS = 453.04) |
| Sub 1-53 | m/z = 370.06(C₂₄H₁₅ClS = 370.89) | Sub 1-54 | m/z = 430.11(C₃₀H₁₉ClO = 430.93) |
| Sub 1-55 | m/z = 444.09(C₃₀H₁₇ClO₂ = 444.91) | Sub 1-56 | m/z = 444.09(C₃₀H₁₇ClO₂ = 444.91) |
| Sub 1-57 | m/z = 470.09(C₃₂H₁₉ClS = 471.01) | Sub 1-58 | m/z = 370.06(C₂₄H₁₅ClS = 370.89) |
| Sub 1-59 | m/z = 412.16(C₂₈H₂₅ClO = 412.96) | Sub 1-60 | m/z = 388.11(C₂₅H₂₁ClS = 388.95) |
| Sub 1-61 | m/z = 354.08(C₂₄H₁₅ClO = 354.83) | Sub 1-62 | m/z = 446.09(C₃₀H₁₉ClS = 446.99) |
| Sub 1-63 | m/z = 370.06(C₂₄H₁₅ClS = 370.89) | Sub 1-64 | m/z = 430.11(C₃₀H₁₉ClO = 430.93) |
| Sub 1-65 | m/z = 370.06(C₂₄H₁₅ClS = 370.89) | Sub 1-66 | m/z = 520.12(C₃₆H₂₁ClO₂ = 521.01) |
| Sub 1-67 | m/z = 459.08(C₃₀H₁₈ClNS = 459.99) | Sub 1-68 | m/z = 370.06(C₂₄H₁₅ClS = 370.89) |
| Sub 1-69 | m/z = 420.07(C₂₈H₁₇ClS = 420.95) | Sub 1-70 | m/z = 354.08(C₂₄H₁₅ClO = 354.83) |
| Sub 1-71 | m/z = 390.18(C₂₆H₂₇ClO = 390.95) | Sub 1-72 | m/z = 370.06(C₂₄H₁₅ClS = 370.89) |
| Sub 1-73 | m/z = 370.06(C₂₄H₁₅ClS = 370.89) | Sub 1-74 | m/z = 370.06(C₂₄H₁₅ClS = 370.89) |
| Sub 1-75 | m/z = 354.08(C₂₄H₁₅ClO = 354.83) | Sub 1-76 | m/z = 354.08(C₂₄H₁₅ClO = 354.83) |
| Sub 1-77 | m/z = 354.08(C₂₄H₁₅ClO = 354.83) | Sub 1-78 | m/z = 360.15(C₂₂H₉D₁₀ClS = 360.97) |
| Sub 1-79 | m/z = 370.06(C₂₄H₁₅ClS = 370.89) | Sub 1-80 | m/z = 388.11(C₂₅H₂₁ClS = 388.95) |
| Sub 1-81 | m/z = 350.09(C₂₂H₁₉ClS = 350.90) | Sub 1-82 | m/z = 406.15(C₂₆H₂₇ClS = 407.01) |
| Sub 1-83 | m/z = 412.16(C₂₈H₂₅ClO = 412.96) | Sub 1-84 | m/z = 334.11(C₂₂H₁₉ClO = 334.84) |
| Sub 1-85 | m/z = 372.13(C₂₅H₂₁ClO = 372.89) | Sub 1-86 | m/z = 430.11(C₃₀H₁₉ClO = 430.93) |
| Sub 1-87 | m/z = 398.09(C₂₆H₁₉ClS = 398.95) | Sub 1-88 | m/z = 355.08(C₂₃H₁₄ClNO = 355.82) |
| Sub 1-89 | m/z = 426.12(C₂₈H₂₃ClS = 427.00) | Sub 1-90 | m/z = 488.19(C₃₄H₂₉ClO = 489.06) |
| Sub 1-91 | m/z = 470.18(C₃₁H₃₁ClS = 471.10) | Sub 1-92 | m/z = 376.11(C₂₄H₂₁ClS = 376.94) |
| Sub 1-93 | m/z = 354.08(C₂₄H₁₅ClO = 354.83) | Sub 1-94 | m/z = 354.08(C₂₄H₁₅ClO = 354.83) |

### Synthesis Example of Sub 2

Sub 2 of Reaction Scheme 1 may be synthesized through the reaction route of Reaction Scheme 3 below, but is not limited thereto.

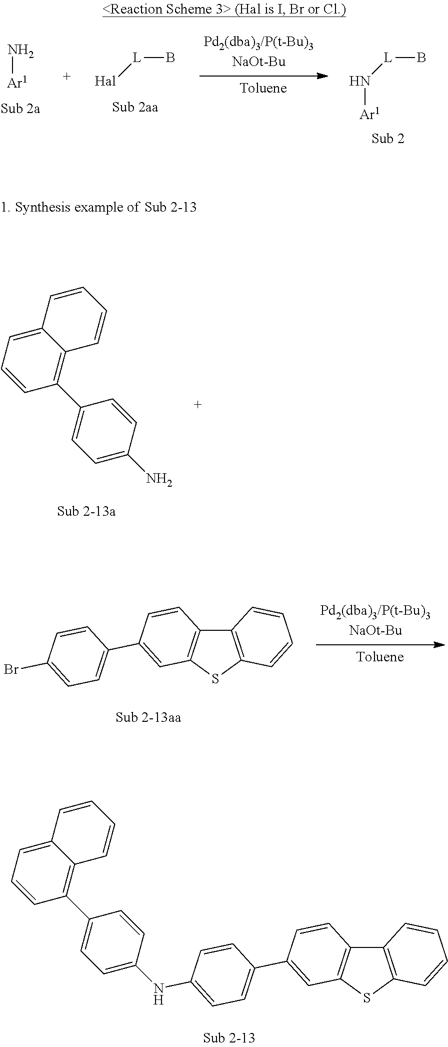

After dissolving Sub 2-13a (50.0 g, 228.0 mmol) in toluene (1140 mL), Sub 2-13b (74.7 g, 228.0 mmol), Pd₂(dba)₃ (6.3 g, 6.8 mmol), P(t-Bu)₃ (2.8 g, 13.7 mmol), and NaOt-Bu (43.8 g, 456.0 mmol) were added to the solution and the mixture was stirred at 120° C. When the reaction was completed, the reaction product was extracted with CH₂Cl₂ and water, and then an organic layer was dried with MgSO₄ and concentrated.

Then, the concentrate was separated through a silica gel column and recrystallized to obtain 80.0 g (yield: 73.5%) of the product.

### 2. Synthesis Example of Sub 2-34

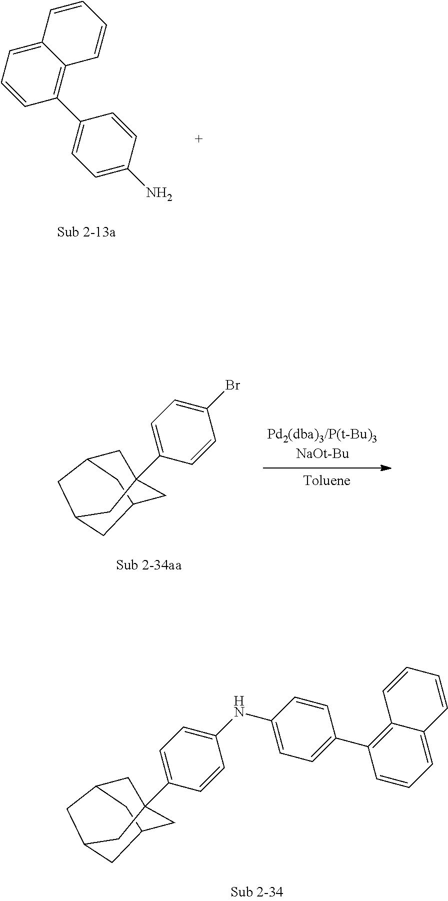 [SMILES: Nc1ccc(-c2cccc3ccccc23)cc1 ; Brc1ccc(C23CC4CC(CC(C4)C2)C3)cc1 ; c1ccc2c(-c3ccc(Nc4ccc(C56CC7CC(CC(C7)C5)C6)cc4)cc3)cccc2c1]

Using Sub 2-13a (50.0 g, 228.0 mmol), Sub 2-34aa (64.2 g, 228.0 mmol), Pd₂(dba)₃ (6.3 g, 6.8 mmol), P(t-Bu)₃ (2.8 g, 13.7 mmol), NaOt-Bu (43.8 g, 456.0 mmol), and toluene (1140 mL), the reaction was carried out in the same manner as the synthesis method of Sub 2-13, and then purified to obtain 72.6 g (yield: 74.1%) of product.

### 3. Synthesis Example of Sub 2-47

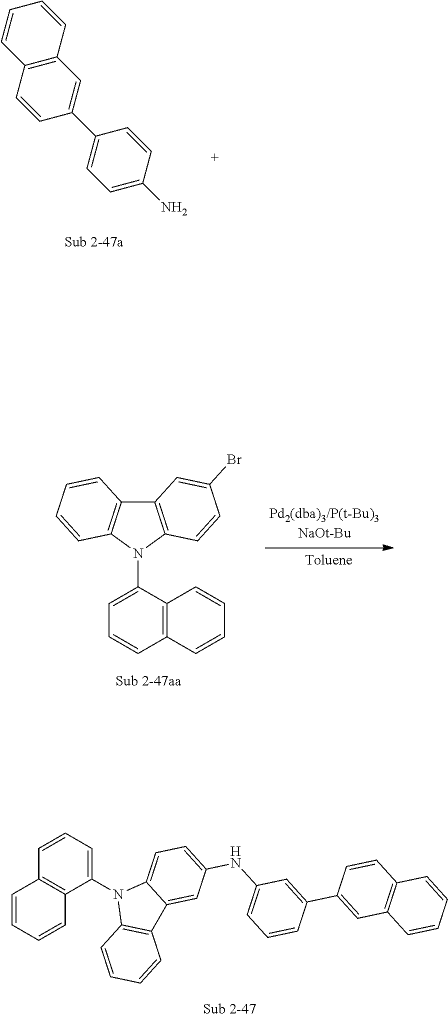 [SMILES: Nc1ccc(-c2ccc3ccccc3c2)cc1 ; Brc1ccc2c(c1)c1ccccc1n2-c1cccc2ccccc12 ; c1cc(Nc2ccc3c(c2)c2ccccc2n3-c2cccc3ccccc23)cc(-c2ccc3ccccc3c2)c1]

Using Sub 2-47a (50.0 g, 228.0 mmol)₄ Sub 2-47aa (82.0 g, 228.0 mmol), Pd₂(dba)₃ (6.3 g, 6.8 mmol), P(t-Bu)₃ (2.8 g, 13.7 mmol), NaOt-Bu (43.8 g, 456.0 mmol), and toluene (1140 mL), the reaction was carried out in the same manner as the synthesis method of Sub 2-13, and then purified to obtain 81.6 g (yield: 70.1%) of product.

### 4. Synthesis Example of Sub 2-86

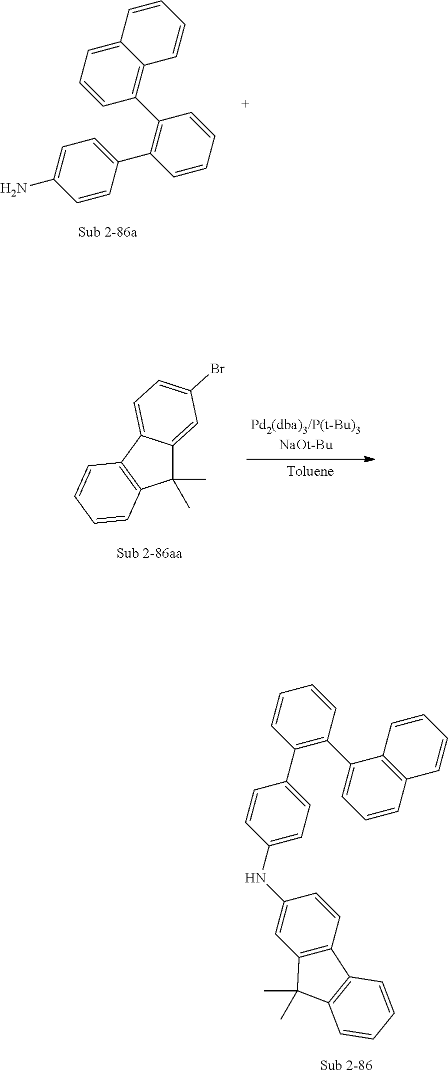 [SMILES: Nc1ccc(-c2ccccc2-c2cccc3ccccc23)cc1 ; CC1(C)c2ccccc2-c2ccc(Br)cc21 ; CC1(C)c2ccccc2-c2ccc(Nc3ccc(-c4ccccc4-c4cccc5ccccc45)cc3)cc21]

Using Sub 2-86a (50.0 g, 169.3 mmol) Sub 2-86aa (44.7 g, 169.3 mmol), Pd₂(dba)₃ (4.7 g, 5.1 mmol), P(t-Bu)₃ (2.1 g, 10.2 mmol), NaOt-Bu (32.5 g, 338.5 mmol), and toluene (846 mL), the reaction was carried out in the same manner as the synthesis method of Sub 2-13, and then purified to obtain 58.9 g (yield: 71.3%) of product.

Compounds belonging to Sub 2 may be, but not limited to, the following compounds, and Table 2 shows FD-MS (Field Desorption-Mass Spectrometry) values of the following compounds.

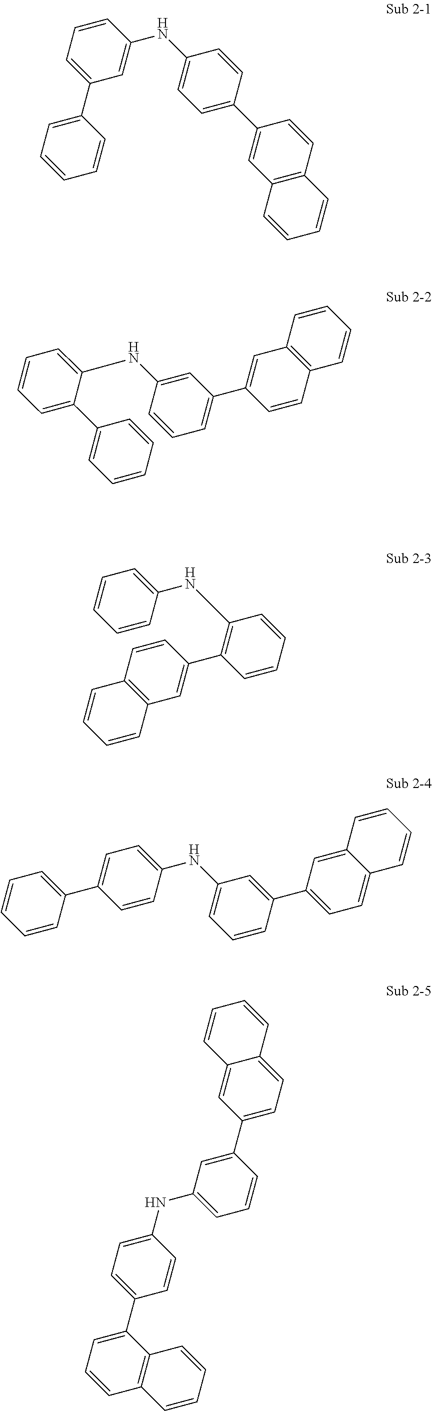 [SMILES: c1ccc(-c2cccc(Nc3ccc(-c4ccc5ccccc5c4)cc3)c2)cc1 ; c1ccc(-c2ccccc2Nc2cccc(-c3ccc4ccccc4c3)c2)cc1 ; c1ccc(Nc2ccccc2-c2ccc3ccccc3c2)cc1 ; c1ccc(-c2ccc(Nc3cccc(-c4ccc5ccccc5c4)c3)cc2)cc1 ; c1cc(Nc2ccc(-c3cccc4ccccc34)cc2)cc(-c2ccc3ccccc3c2)c1]

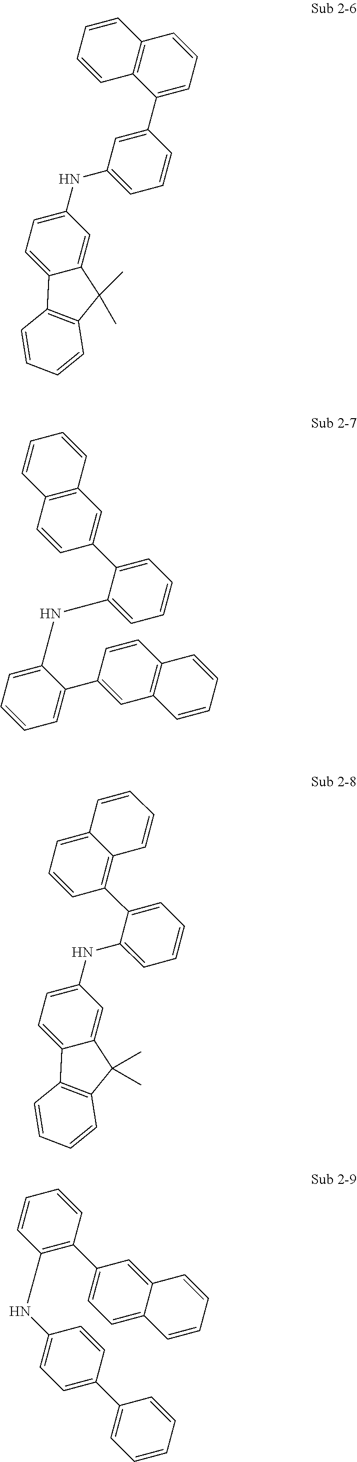

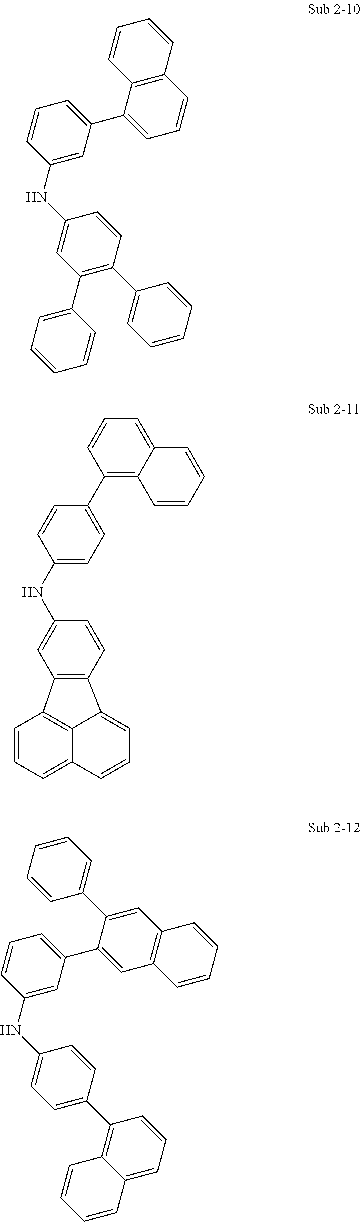 [SMILES: c1ccc(-c2ccc(Nc3cccc(-c4cccc5ccccc45)c3)cc2-c2ccccc2)cc1 ; c1ccc2c(-c3ccc(Nc4ccc5c(c4)-c4cccc6cccc-5c46)cc3)cccc2c1 ; c1ccc(-c2cc3ccccc3cc2-c2cccc(Nc3ccc(-c4cccc5ccccc45)cc3)c2)cc1]

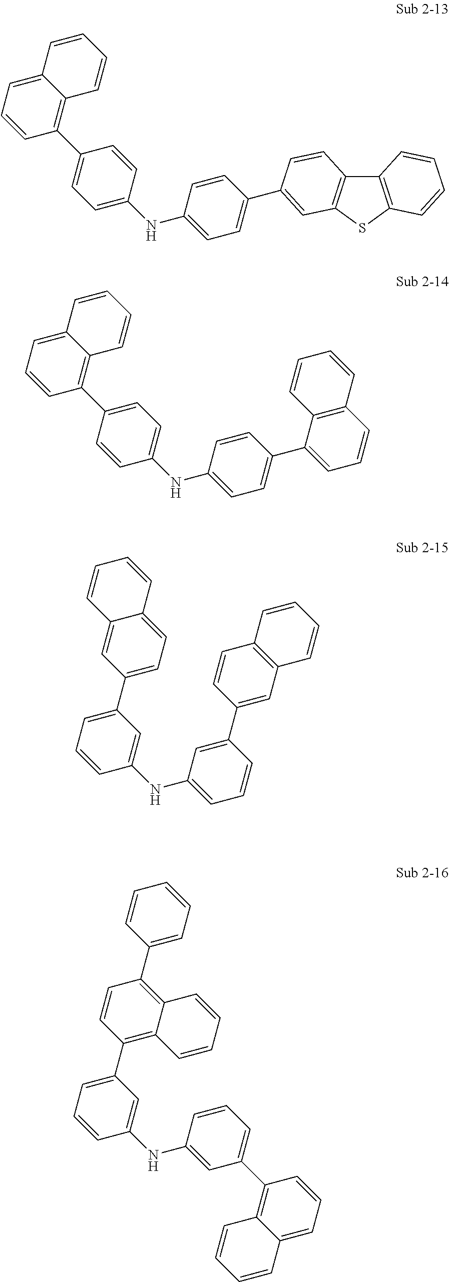 [SMILES: c1ccc2c(-c3ccc(Nc4ccc(-c5ccc6c(c5)sc5ccccc56)cc4)cc3)cccc2c1 ; c1ccc2c(-c3ccc(Nc4ccc(-c5cccc6ccccc56)cc4)cc3)cccc2c1 ; c1cc(Nc2cccc(-c3ccc4ccccc4c3)c2)cc(-c2ccc3ccccc3c2)c1 ; c1ccc(-c2ccc(-c3cccc(Nc4cccc(-c5cccc6ccccc56)c4)c3)c3ccccc23)cc1]

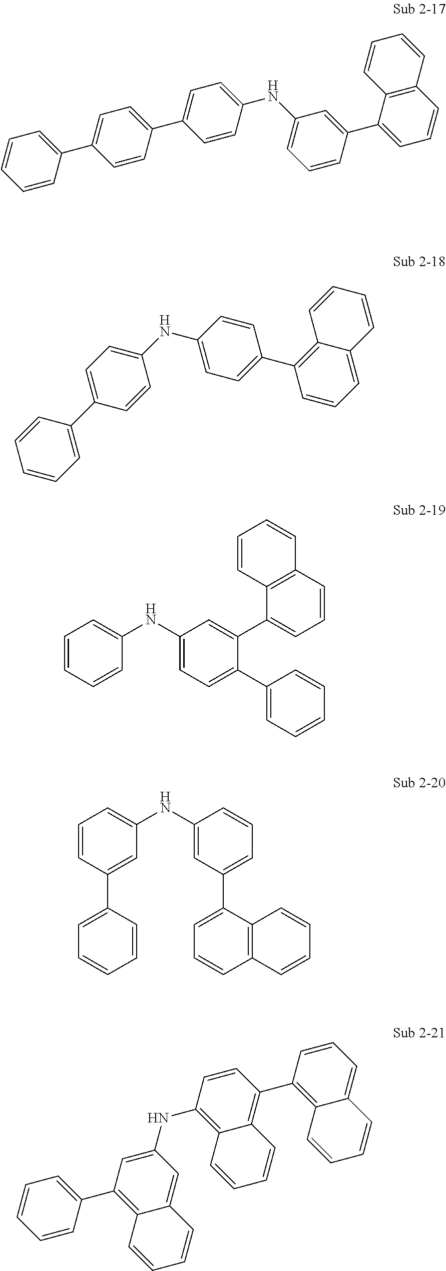 [SMILES: c1ccc(-c2ccc(-c3ccc(Nc4cccc(-c5cccc6ccccc56)c4)cc3)cc2)cc1 ; c1ccc(-c2ccc(Nc3ccc(-c4cccc5ccccc45)cc3)cc2)cc1 ; c1ccc(Nc2ccc(-c3ccccc3)c(-c3cccc4ccccc34)c2)cc1 ; c1ccc(-c2cccc(Nc3cccc(-c4cccc5ccccc45)c3)c2)cc1 ; c1ccc(-c2cc(Nc3ccc(-c4cccc5ccccc45)c4ccccc34)cc3ccccc23)cc1]

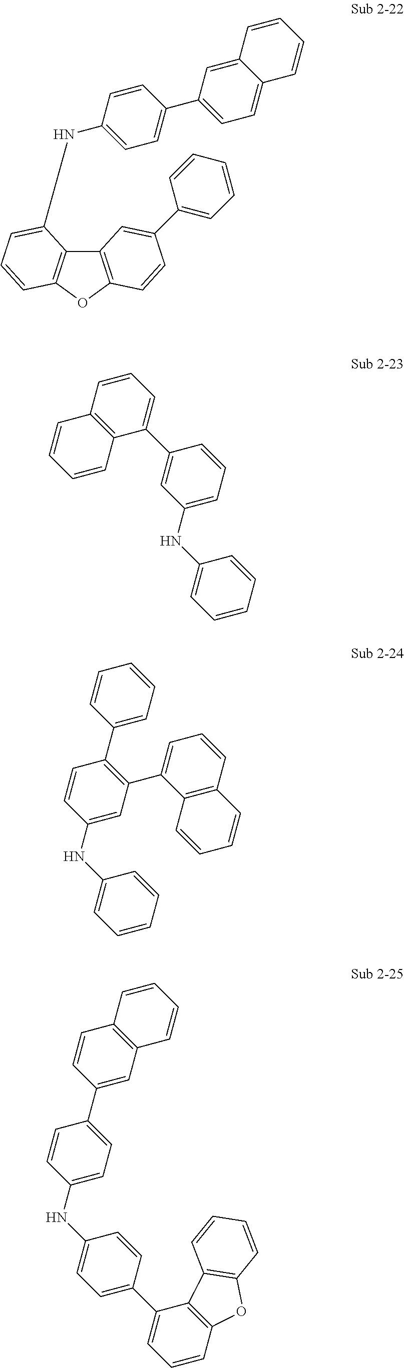 [SMILES: c1ccc(-c2ccc3oc4cccc(Nc5ccc(-c6ccc7ccccc7c6)cc5)c4c3c2)cc1 ; c1ccc(Nc2cccc(-c3cccc4ccccc34)c2)cc1 ; c1ccc(Nc2ccc(-c3ccccc3)c(-c3cccc4ccccc34)c2)cc1 ; c1ccc2cc(-c3ccc(Nc4ccc(-c5cccc6oc7ccccc7c56)cc4)cc3)ccc2c1]

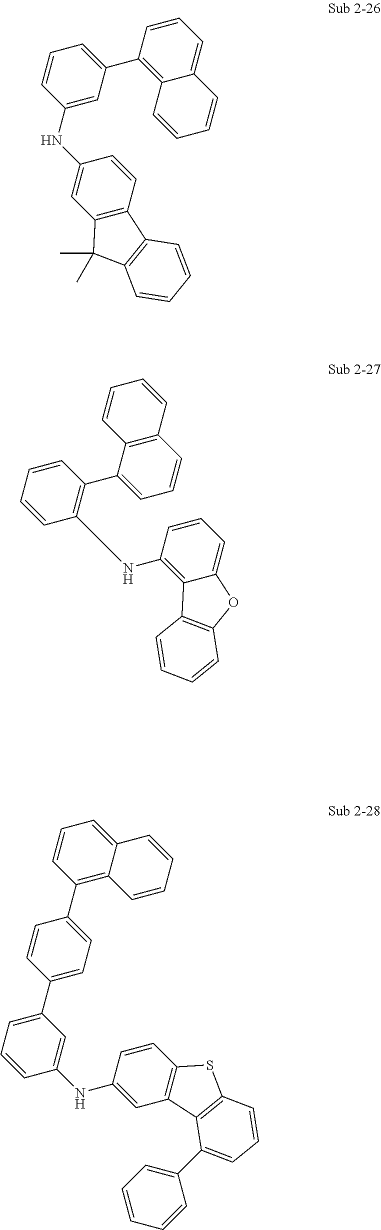 [SMILES: CC1(C)c2ccccc2-c2ccc(Nc3cccc(-c4cccc5ccccc45)c3)cc21 ; c1ccc(-c2cccc3ccccc23)c(Nc2cccc3oc4ccccc4c23)c1 ; c1ccc(-c2cccc3sc4ccc(Nc5cccc(-c6ccc(-c7cccc8ccccc78)cc6)c5)cc4c23)cc1]

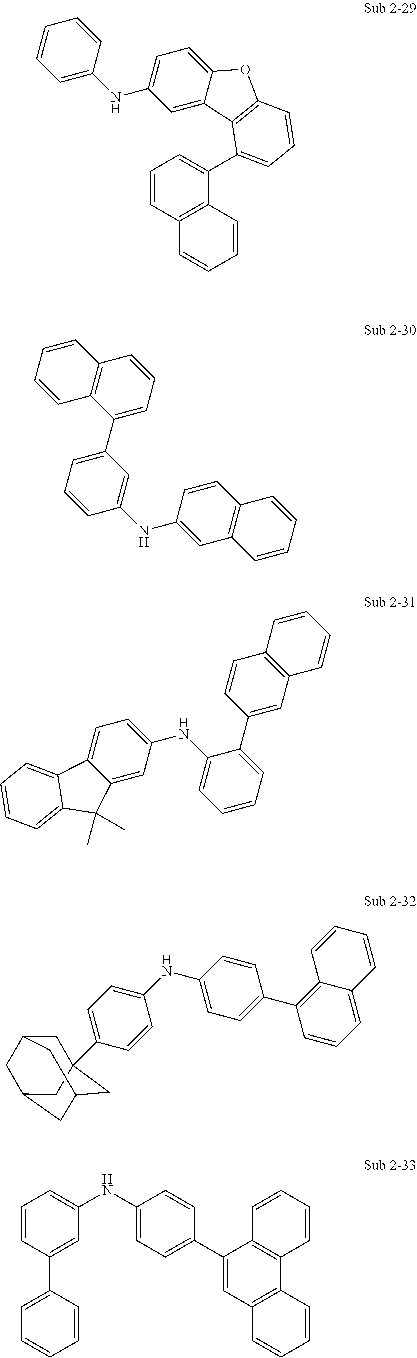 [SMILES: c1ccc(Nc2ccc3oc4cccc(-c5cccc6ccccc56)c4c3c2)cc1 ; c1cc(Nc2ccc3ccccc3c2)cc(-c2cccc3ccccc23)c1 ; CC1(C)c2ccccc2-c2ccc(Nc3ccccc3-c3ccc4ccccc4c3)cc21 ; c1ccc2c(-c3ccc(Nc4ccc(C56CC7CC(CC(C7)C5)C6)cc4)cc3)cccc2c1 ; c1ccc(-c2cccc(Nc3ccc(-c4cc5ccccc5c5ccccc45)cc3)c2)cc1]

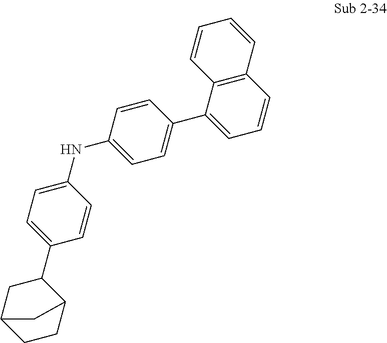 [SMILES: c1ccc2c(-c3ccc(Nc4ccc(C5CC6CCC5C6)cc4)cc3)cccc2c1]

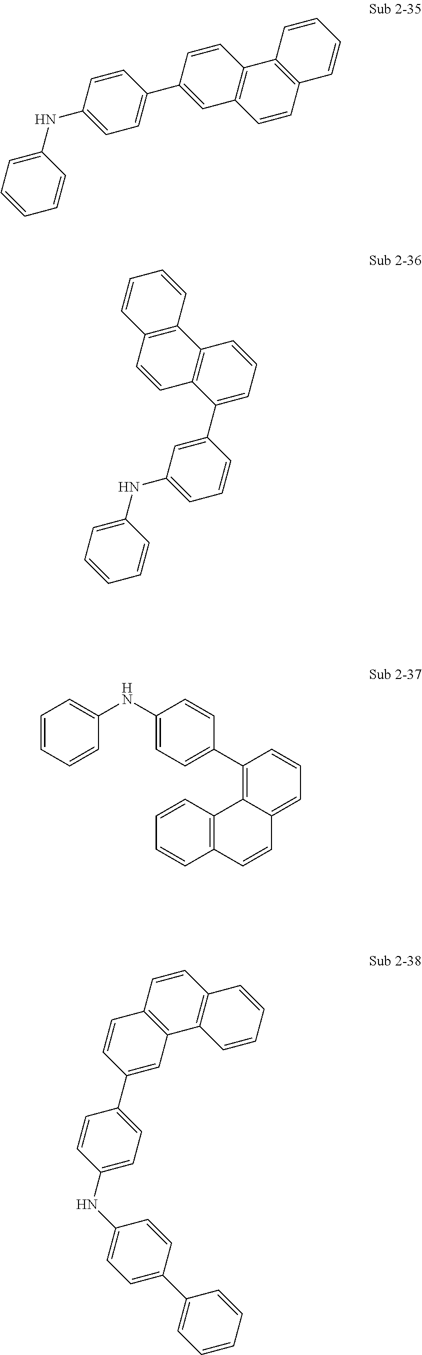 [SMILES: c1ccc(Nc2ccc(-c3ccc4c(ccc5ccccc54)c3)cc2)cc1 ; c1ccc(Nc2cccc(-c3cccc4c3ccc3ccccc34)c2)cc1 ; c1ccc(Nc2ccc(-c3cccc4ccc5ccccc5c34)cc2)cc1 ; c1ccc(-c2ccc(Nc3ccc(-c4ccc5ccc6ccccc6c5c4)cc3)cc2)cc1]

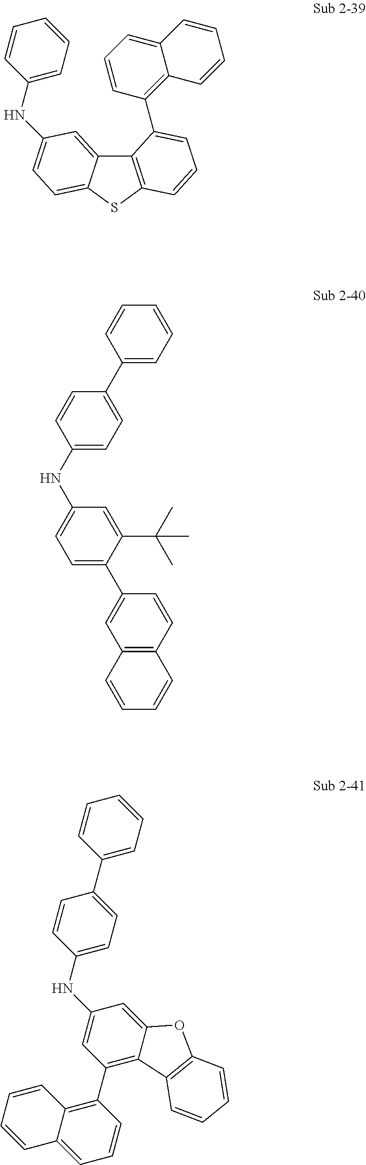 [SMILES: c1ccc(Nc2ccc3sc4cccc(-c5cccc6ccccc56)c4c3c2)cc1 ; CC(C)(C)c1cc(Nc2ccc(-c3ccccc3)cc2)ccc1-c1ccc2ccccc2c1 ; c1ccc(-c2ccc(Nc3cc(-c4cccc5ccccc45)c4c(c3)oc3ccccc34)cc2)cc1]

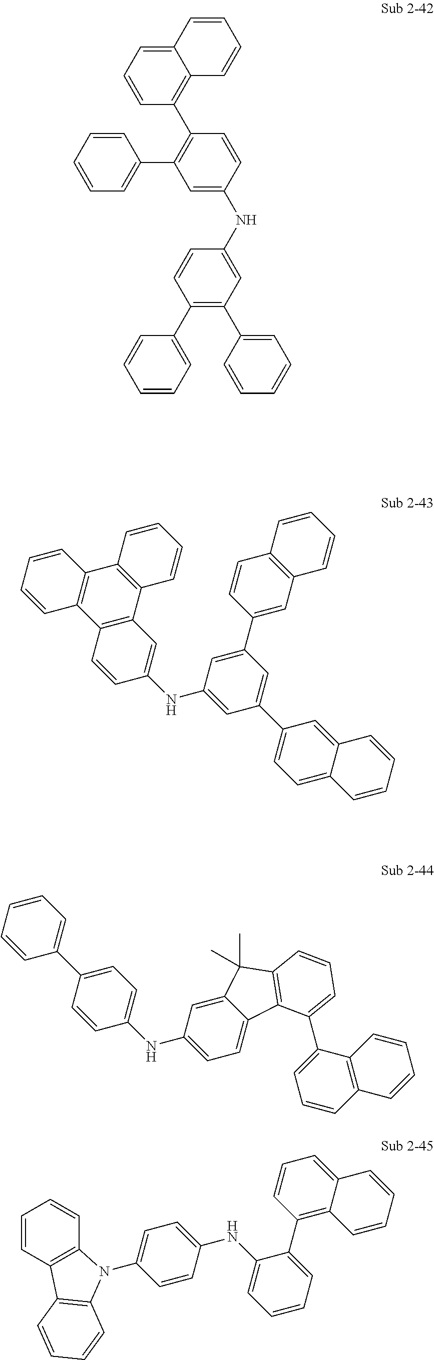 [SMILES: c1ccc(-c2ccc(Nc3ccc(-c4cccc5ccccc45)c(-c4ccccc4)c3)cc2-c2ccccc2)cc1 ; c1ccc2cc(-c3cc(Nc4ccc5c6ccccc6c6ccccc6c5c4)cc(-c4ccc5ccccc5c4)c3)ccc2c1 ; CC1(C)c2cc(Nc3ccc(-c4ccccc4)cc3)ccc2-c2c(-c3cccc4ccccc34)cccc21 ; c1ccc(-c2cccc3ccccc23)c(Nc2ccc(-n3c4ccccc4c4ccccc43)cc2)c1]

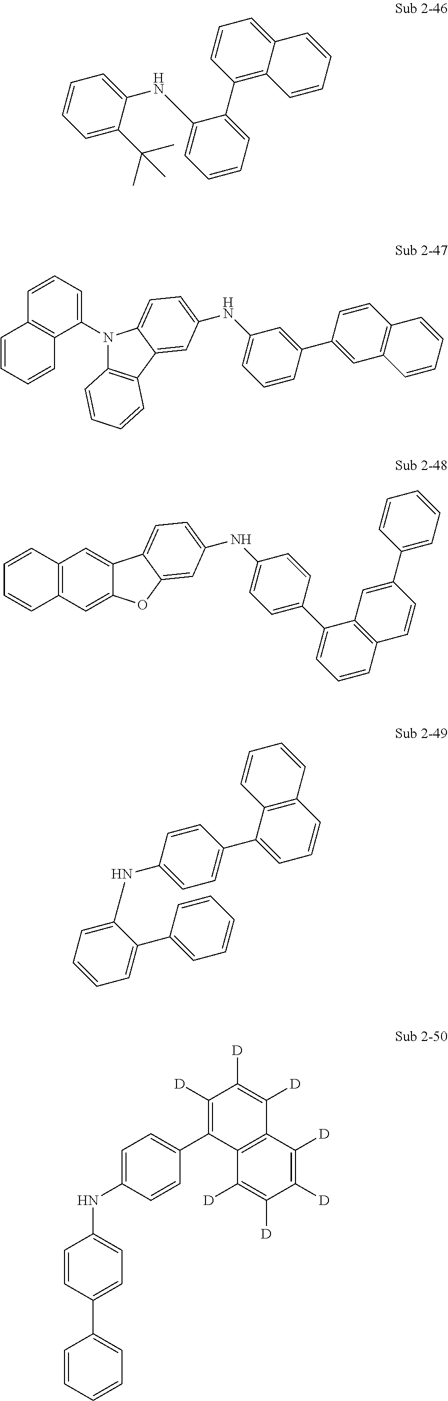 [SMILES: CC(C)(C)c1ccccc1Nc1ccccc1-c1cccc2ccccc12 ; c1cc(Nc2ccc3c(c2)c2ccccc2n3-c2cccc3ccccc23)cc(-c2ccc3ccccc3c2)c1 ; c1ccc(-c2ccc3cccc(-c4ccc(Nc5ccc6c(c5)oc5cc7ccccc7cc56)cc4)c3c2)cc1 ; c1ccc(-c2ccccc2Nc2ccc(-c3cccc4ccccc34)cc2)cc1 ; [2H]c1c([2H])c([2H])c2c(-c3ccc(Nc4ccc(-c5ccccc5)cc4)cc3)c([2H])c([2H])c([2H])c2c1[2H]]

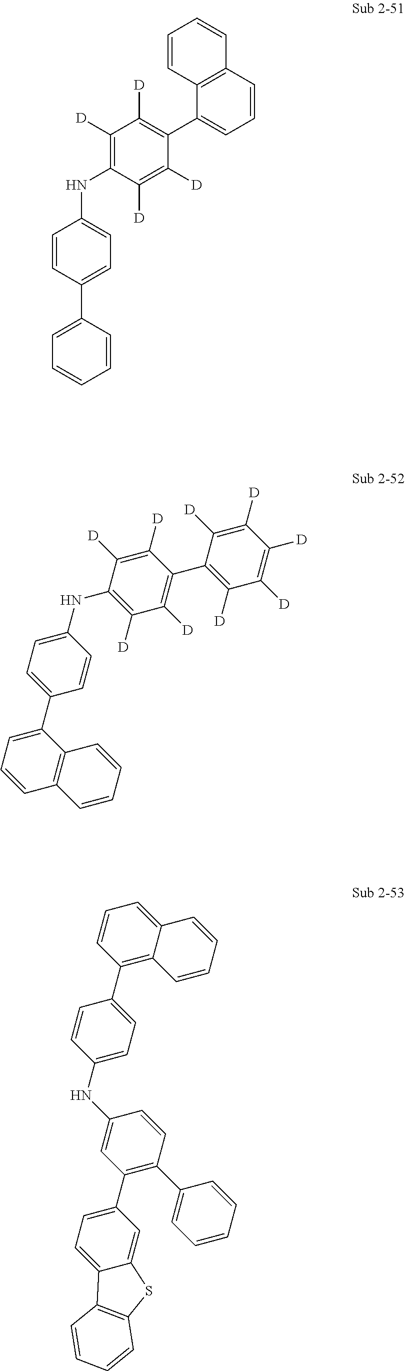 [SMILES: [2H]c1c([2H])c(-c2cccc3ccccc23)c([2H])c([2H])c1Nc1ccc(-c2ccccc2)cc1 ; [2H]c1c([2H])c([2H])c(-c2c([2H])c([2H])c(Nc3ccc(-c4cccc5ccccc45)cc3)c([2H])c2[2H])c([2H])c1[2H] ; c1ccc(-c2ccc(Nc3ccc(-c4cccc5ccccc45)cc3)cc2-c2ccc3c(c2)sc2ccccc23)cc1]

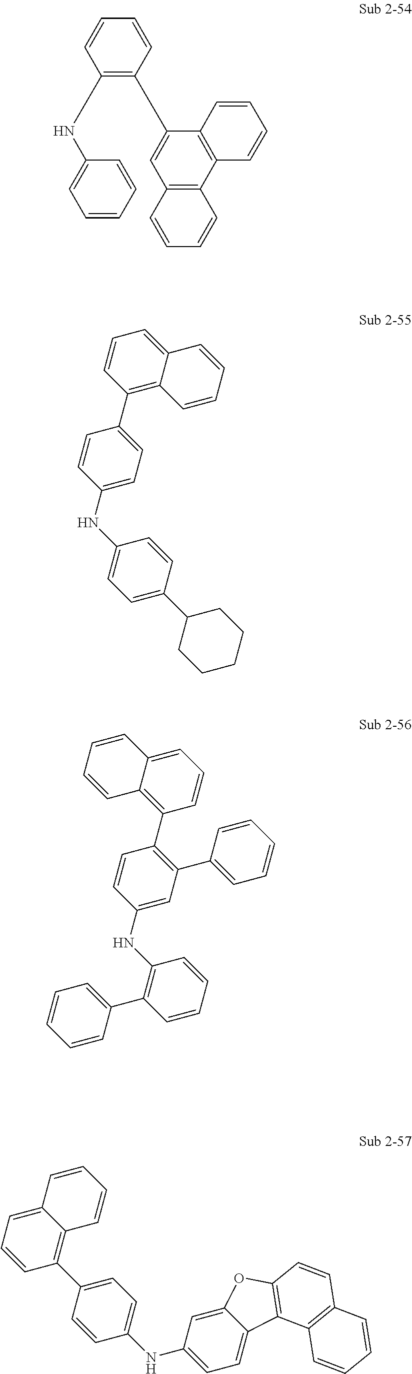 [SMILES: c1ccc(Nc2ccccc2-c2cc3ccccc3c3ccccc23)cc1 ; c1ccc2c(-c3ccc(Nc4ccc(C5CCCCC5)cc4)cc3)cccc2c1 ; c1ccc(-c2ccccc2Nc2ccc(-c3cccc4ccccc34)c(-c3ccccc3)c2)cc1 ; c1ccc2c(-c3ccc(Nc4ccc5c(c4)oc4ccc6ccccc6c45)cc3)cccc2c1]

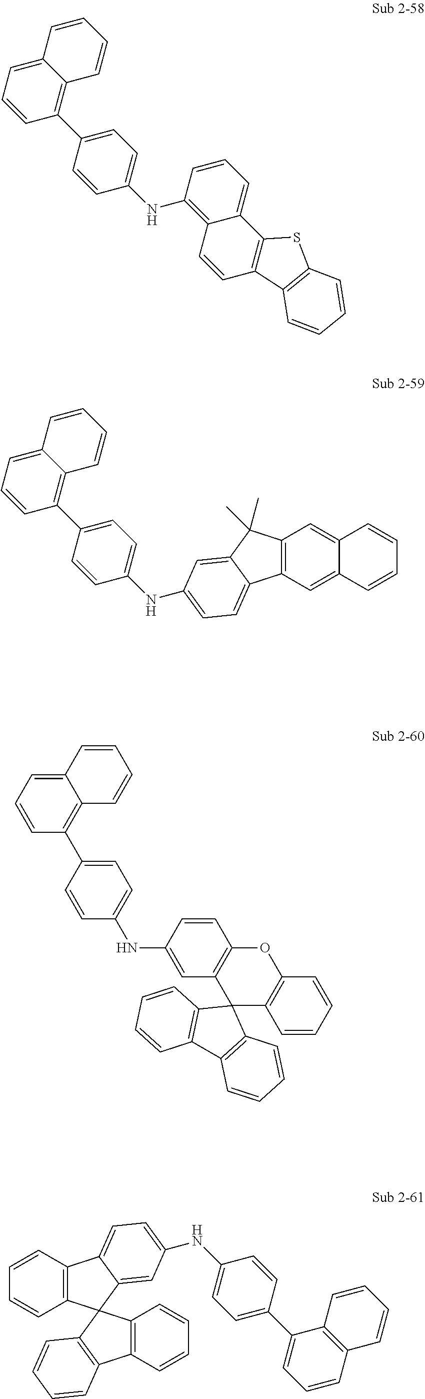 [SMILES: c1ccc2c(-c3ccc(Nc4cccc5c4ccc4c6ccccc6sc54)cc3)cccc2c1 ; CC1(C)c2cc(Nc3ccc(-c4cccc5ccccc45)cc3)ccc2-c2cc3ccccc3cc21 ; c1ccc2c(c1)Oc1ccc(Nc3ccc(-c4cccc5ccccc45)cc3)cc1C21c2ccccc2-c2ccccc21 ; c1ccc2c(c1)-c1ccccc1C21c2ccccc2-c2ccc(Nc3ccc(-c4cccc5ccccc45)cc3)cc21]

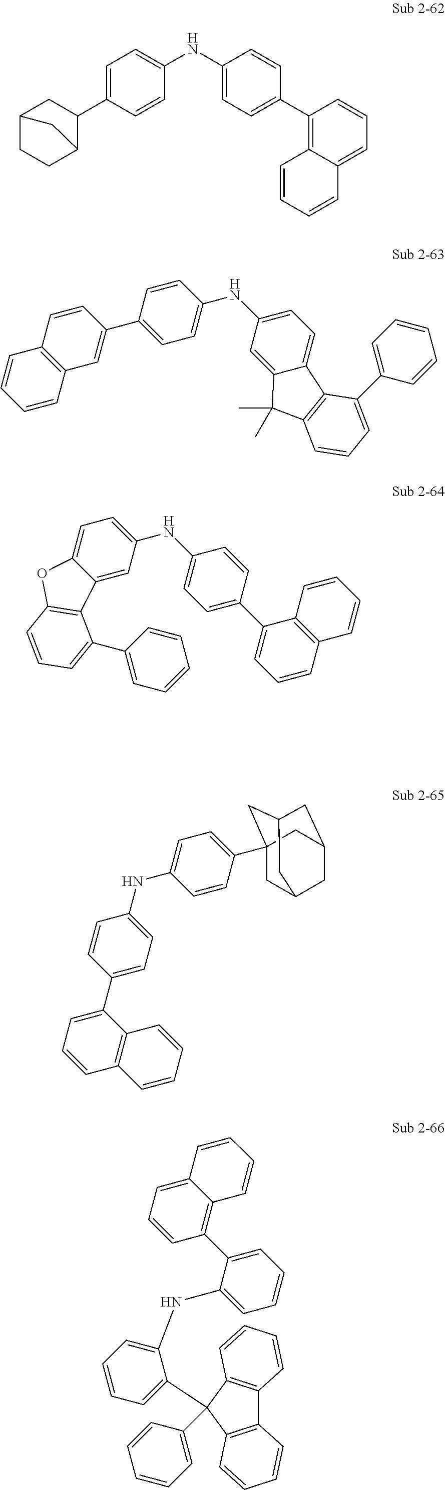 [SMILES: c1ccc2c(-c3ccc(Nc4ccc(C5CC6CCC5C6)cc4)cc3)cccc2c1 ; CC1(C)c2cc(Nc3ccc(-c4ccc5ccccc5c4)cc3)ccc2-c2c(-c3ccccc3)cccc21 ; c1ccc(-c2cccc3oc4ccc(Nc5ccc(-c6cccc7ccccc67)cc5)cc4c23)cc1 ; c1ccc2c(-c3ccc(Nc4ccc(C56CC7CC(CC(C7)C5)C6)cc4)cc3)cccc2c1 ; c1ccc(C2(c3ccccc3Nc3ccccc3-c3cccc4ccccc34)c3ccccc3-c3ccccc32)cc1]

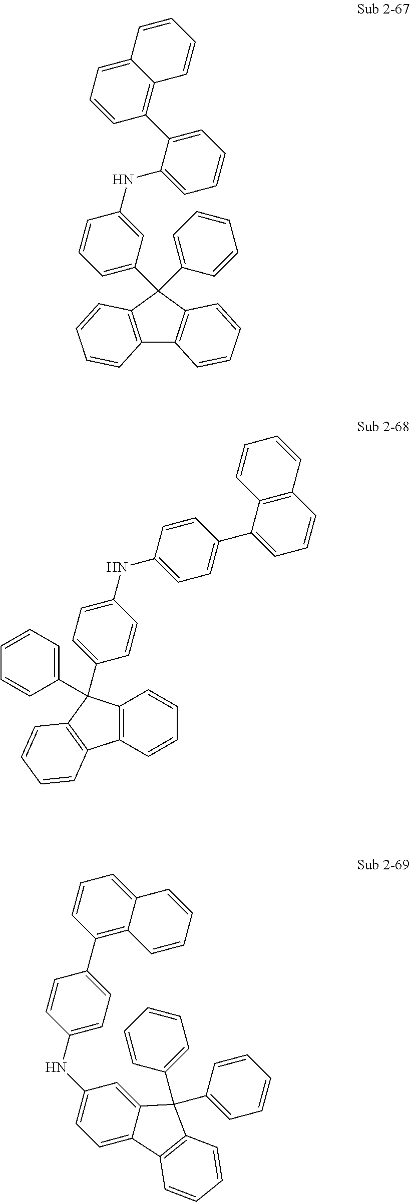 [SMILES: c1ccc(C2(c3cccc(Nc4ccccc4-c4cccc5ccccc45)c3)c3ccccc3-c3ccccc32)cc1 ; c1ccc(C2(c3ccc(Nc4ccc(-c5cccc6ccccc56)cc4)cc3)c3ccccc3-c3ccccc32)cc1 ; c1ccc(C2(c3ccccc3)c3ccccc3-c3ccc(Nc4ccc(-c5cccc6ccccc56)cc4)cc32)cc1]

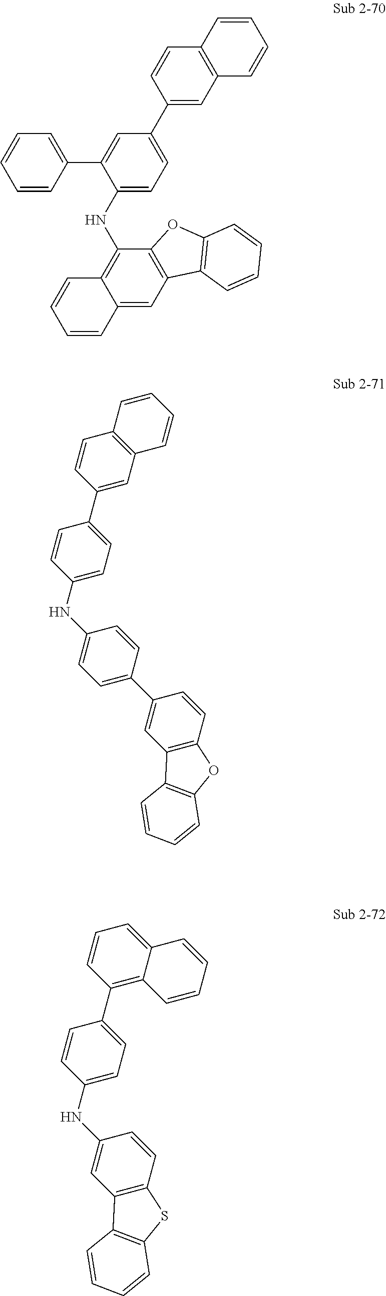 [SMILES: c1ccc(-c2cc(-c3ccc4ccccc4c3)ccc2Nc2c3ccccc3cc3c2oc2ccccc23)cc1 ; c1ccc2cc(-c3ccc(Nc4ccc(-c5ccc6oc7ccccc7c6c5)cc4)cc3)ccc2c1 ; c1ccc2c(-c3ccc(Nc4ccc5sc6ccccc6c5c4)cc3)cccc2c1]

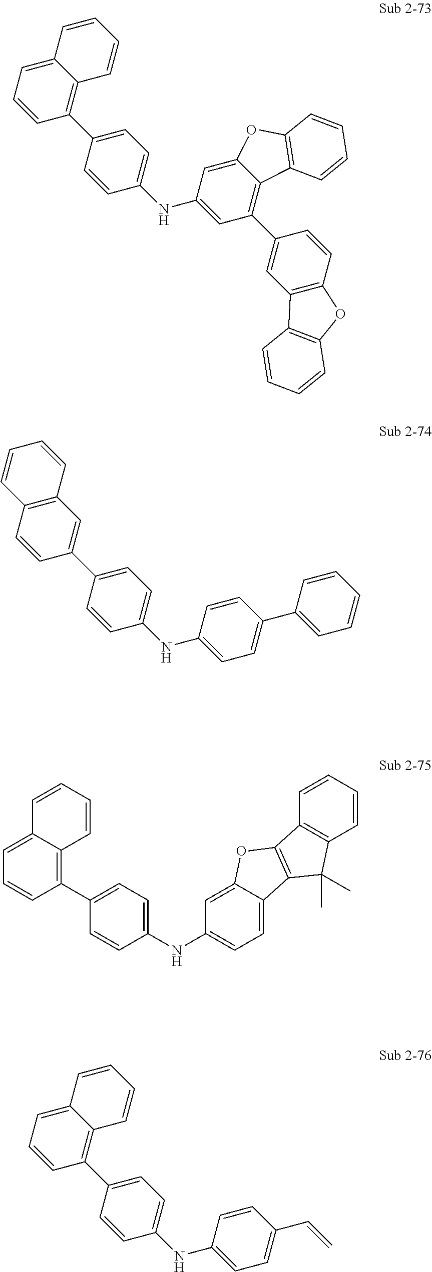 [SMILES: c1ccc2c(-c3ccc(Nc4cc(-c5ccc6oc7ccccc7c6c5)c5c(c4)oc4ccccc45)cc3)cccc2c1 ; c1ccc(-c2ccc(Nc3ccc(-c4ccc5ccccc5c4)cc3)cc2)cc1 ; CC1(C)c2ccccc2-c2oc3cc(Nc4ccc(-c5cccc6ccccc56)cc4)ccc3c21 ; C=Cc1ccc(Nc2ccc(-c3cccc4ccccc34)cc2)cc1]

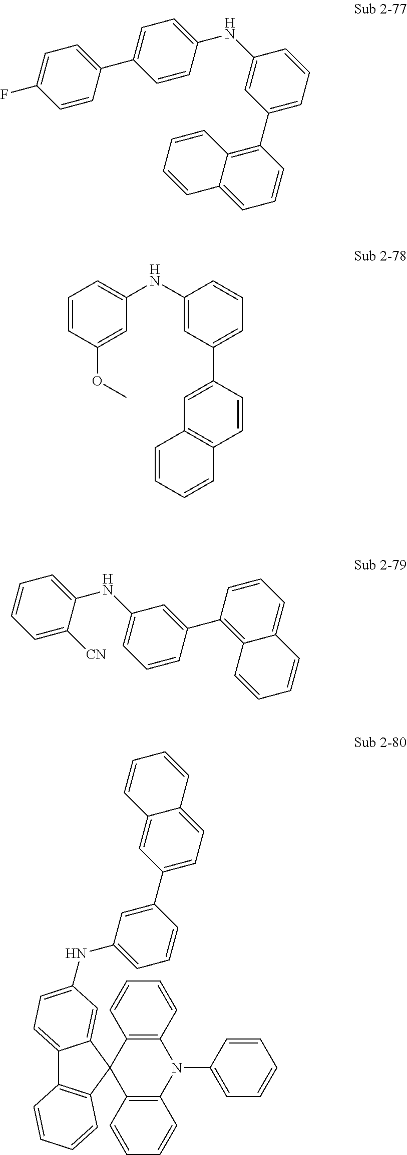 [SMILES: Fc1ccc(-c2ccc(Nc3cccc(-c4cccc5ccccc45)c3)cc2)cc1 ; COc1cccc(Nc2cccc(-c3ccc4ccccc4c3)c2)c1 ; N#Cc1ccccc1Nc1cccc(-c2cccc3ccccc23)c1 ; c1ccc(N2c3ccccc3C3(c4ccccc4-c4ccc(Nc5cccc(-c6ccc7ccccc7c6)c5)cc43)c3ccccc32)cc1]

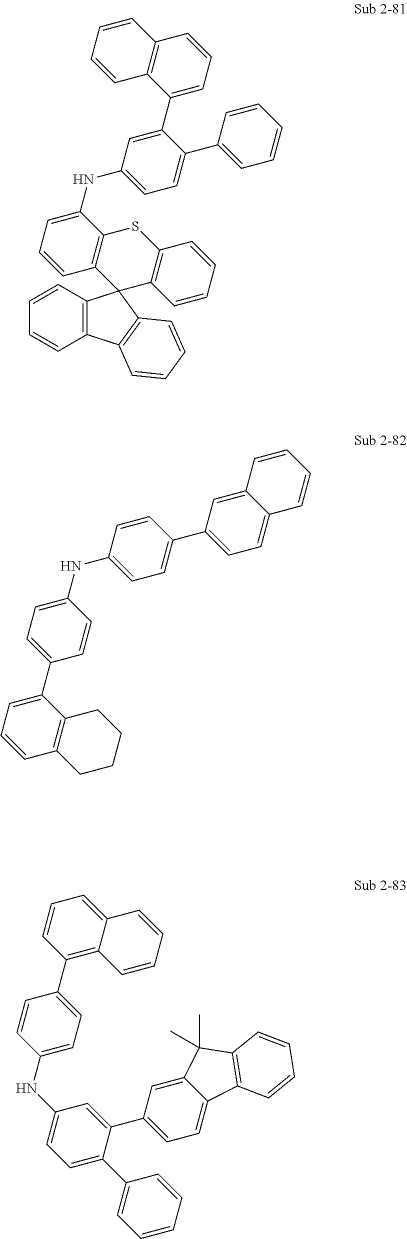 [SMILES: c1ccc(-c2ccc(Nc3cccc4c3Sc3ccccc3C43c4ccccc4-c4ccccc43)cc2-c2cccc3ccccc23)cc1 ; c1cc2c(c(-c3ccc(Nc4ccc(-c5ccc6ccccc6c5)cc4)cc3)c1)CCCC2 ; CC1(C)c2ccccc2-c2ccc(-c3cc(Nc4ccc(-c5cccc6ccccc56)cc4)ccc3-c3ccccc3)cc21]

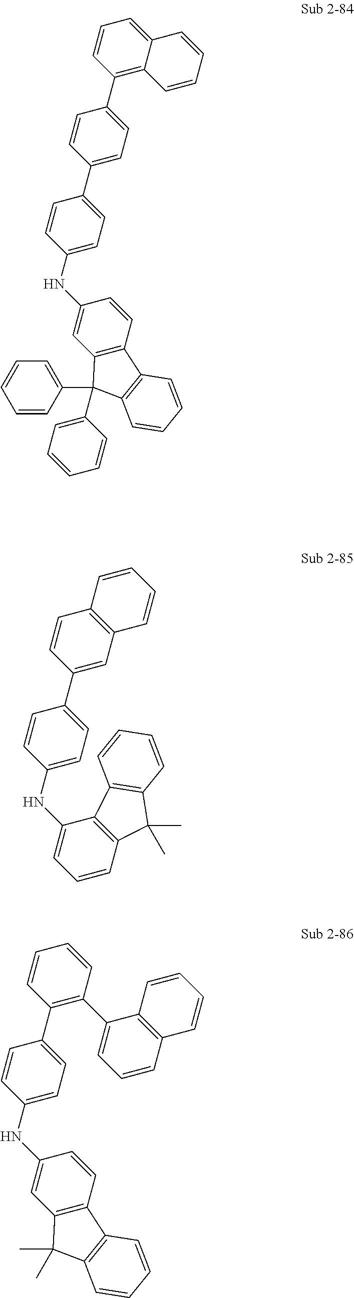 [SMILES: c1ccc(C2(c3ccccc3)c3ccccc3-c3ccc(Nc4ccc(-c5ccc(-c6cccc7ccccc67)cc5)cc4)cc32)cc1 ; CC1(C)c2ccccc2-c2c(Nc3ccc(-c4ccc5ccccc5c4)cc3)cccc21 ; CC1(C)c2ccccc2-c2ccc(Nc3ccc(-c4ccccc4-c4cccc5ccccc45)cc3)cc21]

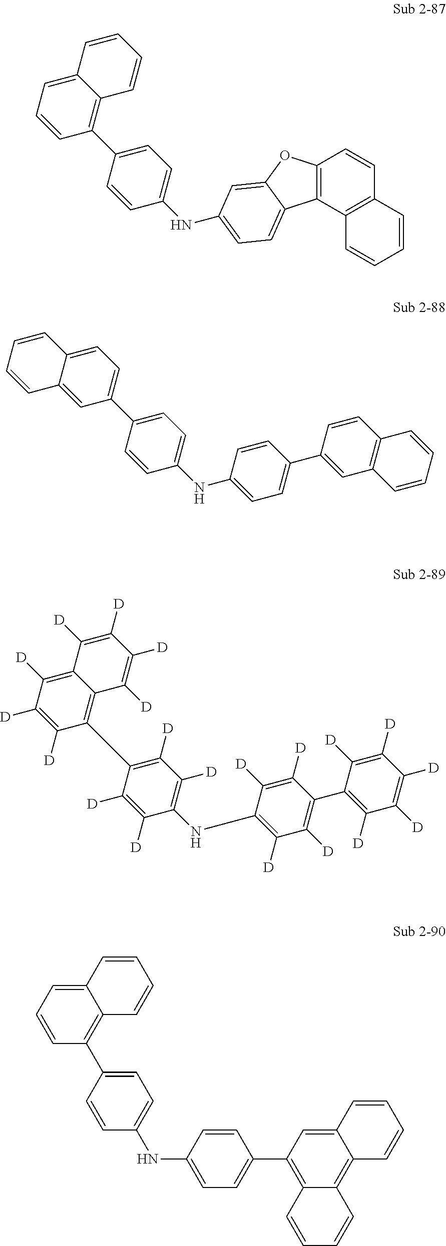 [SMILES: c1ccc2c(-c3ccc(Nc4ccc5c(c4)oc4ccc6ccccc6c45)cc3)cccc2c1 ; c1ccc2cc(-c3ccc(Nc4ccc(-c5ccc6ccccc6c5)cc4)cc3)ccc2c1 ; [2H]c1c([2H])c([2H])c(-c2c([2H])c([2H])c(Nc3c([2H])c([2H])c(-c4c([2H])c([2H])c([2H])c5c([2H])c([2H])c([2H])c([2H])c45)c([2H])c3[2H])c([2H])c2[2H])c([2H])c1[2H] ; c1ccc2c(-c3ccc(Nc4ccc(-c5cc6ccccc6c6ccccc56)cc4)cc3)cccc2c1]

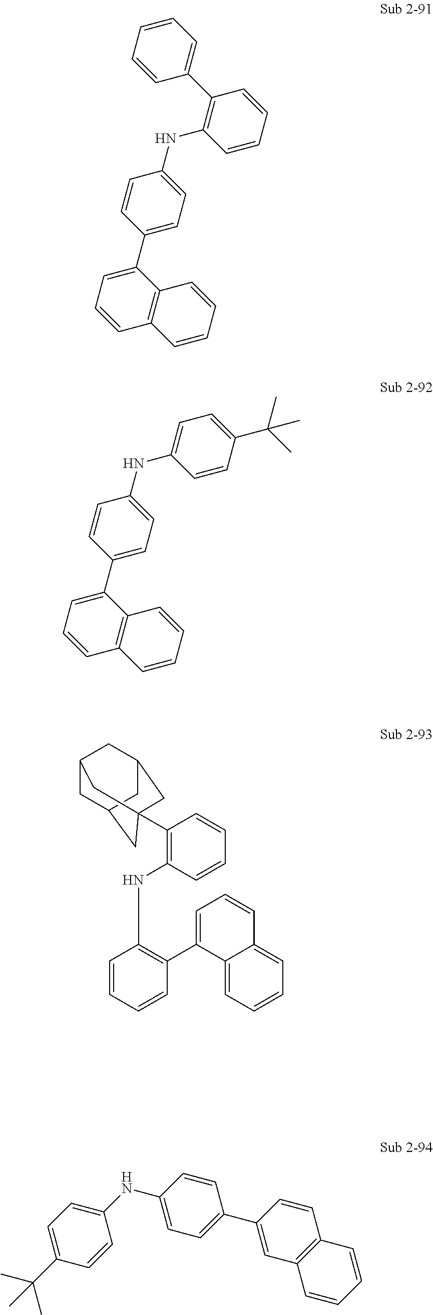

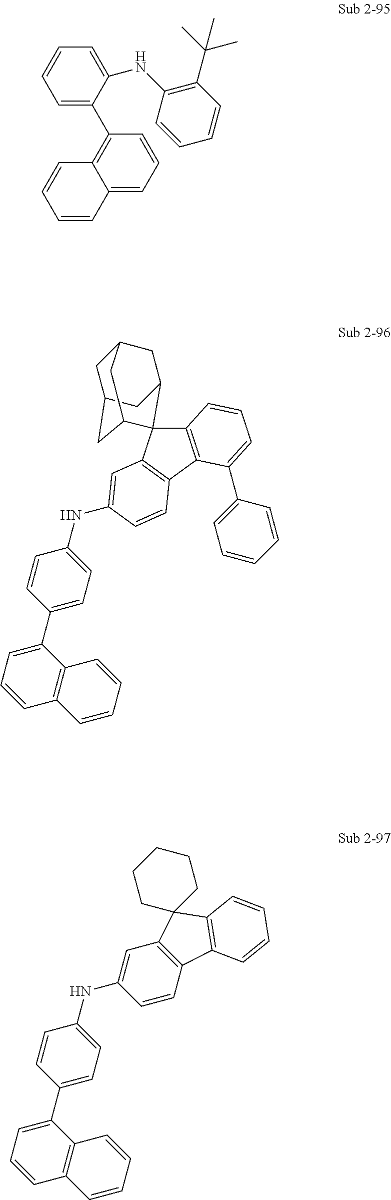 [SMILES: CC(C)(C)c1ccccc1Nc1ccccc1-c1cccc2ccccc12 ; c1ccc(-c2cccc3c2-c2ccc(Nc4ccc(-c5cccc6ccccc56)cc4)cc2C32C3CC4CC(C3)CC2C4)cc1 ; c1ccc2c(c1)-c1ccc(Nc3ccc(-c4cccc5ccccc45)cc3)cc1C21CCCCC1]

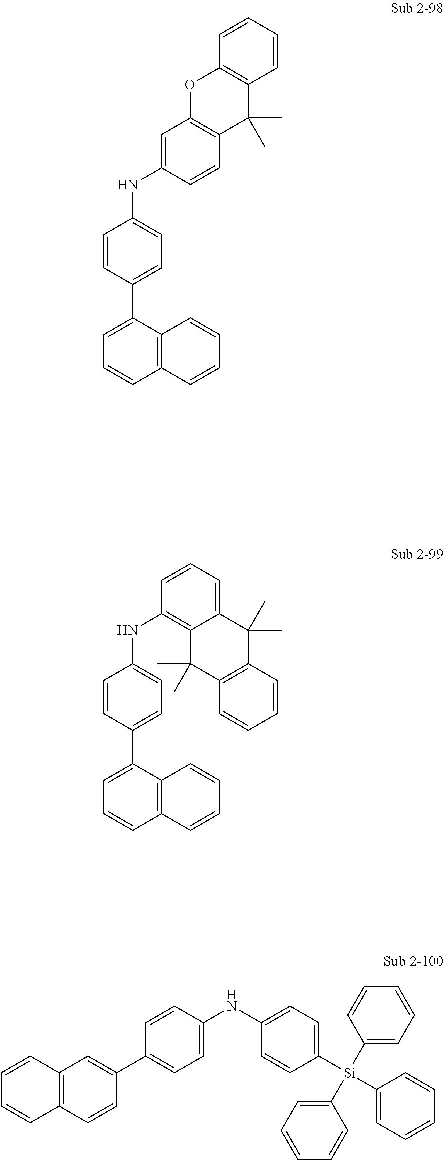 [SMILES: CC1(C)c2ccccc2Oc2cc(Nc3ccc(-c4cccc5ccccc45)cc3)ccc21 ; CC1(C)c2ccccc2C(C)(C)c2c(Nc3ccc(-c4cccc5ccccc45)cc3)cccc21 ; c1ccc([Si](c2ccccc2)(c2ccccc2)c2ccc(Nc3ccc(-c4ccc5ccccc5c4)cc3)cc2)cc1]

TABLE 2

| Compound | FD-MS | Compound | FD-MS |
|---|---|---|---|
| Sub 2-1 | m/z = 371.17(C₂₈H₂₁N = 371.48) | Sub 2-2 | m/z = 371.17(C₂₈H₂₁N = 371.48) |
| Sub 2-3 | m/z = 295.14(C₂₂H₁₇N = 295.39) | Sub 2-4 | m/z = 371.17(C₂₈H₂₁N = 371.48) |
| Sub 2-5 | m/z = 421.54(C₃₂H₂₃N = 421.54) | Sub 2-6 | m/z = 411.20(C₃₁H₂₅N = 411.55) |
| Sub 2-7 | m/z = 421.18(C₃₂H₂₃N = 421.54) | Sub 2-8 | m/z = 411.20(C₃₁H₂₅N = 411.55) |
| Sub 2-9 | m/z = 371.17(C₂₈H₂₁N = 371.48) | Sub 2-10 | m/z = 447.20(C₃₄H₂₅N = 447.58) |
| Sub 2-11 | m/z = 419.17(C₃₂H₂₁N = 419.53) | Sub 2-12 | m/z = 497.21(C₃₈H₂₇N = 497.64) |
| Sub 2-13 | m/z = 477.16(C₃₄H₂₃NS = 477.63) | Sub 2-14 | m/z = 421.18(C₃₂H₂₃N = 421.54) |
| Sub 2-15 | m/z = 421.18(C₃₂H₂₃N = 421.54) | Sub 2-16 | m/z = 497.21(C₃₈H₂₇N = 497.64) |
| Sub 2-17 | m/z = 447.20(C₃₄H₂₅N = 447.58) | Sub 2-18 | m/z = 371.17(C₂₈H₂₁N = 371.48) |
| Sub 2-19 | m/z = 371.17(C₂₈H₂₁N = 371.48) | Sub 2-20 | m/z = 371.17(C₂₈H₂₁N = 371.48) |
| Sub 2-21 | m/z = 471.20(C₃₆H₂₅N = 471.60) | Sub 2-22 | m/z = 461.18(C₃₄H₂₃NO = 461.56) |
| Sub 2-23 | m/z = 295.14(C₂₂H₁₇N = 295.39) | Sub 2-24 | m/z = 371.17(C₂₈H₂₁N = 371.48) |
| Sub 2-25 | m/z = 461.18(C₃₄H₂₃NO = 461.56) | Sub 2-26 | m/z = 411.2(C₃₁H₂₅N = 411.55) |
| Sub 2-27 | m/z = 385.15(C₂₈H₁₉NO = 385.47) | Sub 2-28 | m/z = 553.19(C₄₀H₂₇NS = 553.72) |
| Sub 2-29 | m/z = 385.15(C₂₈H₁₉NO = 385.47) | Sub 2-30 | m/z = 345.15(C₂₆H₁₉N = 345.45) |
| Sub 2-31 | m/z = 411.20(C₃₁H₂₅N = 411.55) | Sub 2-32 | m/z = 429.25(C₃₂H₃₁N = 429.61) |
| Sub 2-33 | m/z = 421.18(C₃₂H₂₃N = 421.54) | Sub 2-34 | m/z = 389.21(C₂₉H₂₇N = 389.54) |
| Sub 2-35 | m/z = 345.15(C₂₆H₁₉N = 345.45) | Sub 2-36 | m/z = 345.15(C₂₆H₁₉N = 345.45) |
| Sub 2-37 | m/z = 345.15(C₂₆H₁₉N = 345.45) | Sub 2-38 | m/z = 421.18(C₃₂H₂₃N = 421.54) |
| Sub 2-39 | m/z = 401.12(C₂₈H₁₉NS = 401.53) | Sub 2-40 | m/z = 427.23(C₃₂H₂₉N = 427.59) |
| Sub 2-41 | m/z = 461.18(C₃₄H₂₃NO = 461.56) | Sub 2-42 | m/z = 523.23(C₄₀H₂₉N = 523.68) |
| Sub 2-43 | m/z = 571.23(C₄₄H₂₉N = 571.72) | Sub 2-44 | m/z = 487.23(C₃₇H₂₉N = 487.65) |
| Sub 2-45 | m/z = 460.19(C₃₄H₂₄N₂ = 460.58) | Sub 2-46 | m/z = 351.20(C₂₆H₂₅N = 351.49) |
| Sub 2-47 | m/z = 510.21(C₃₈H₂₆N₂ = 510.64) | Sub 2-48 | m/z = 511.19(C₃₈H₂₅NO = 511.62) |
| Sub 2-49 | m/z = 371.17(C₂₈H₂₁N = 371.48) | Sub 2-50 | m/z = 378.21(C₂₈H₁₄D₇N = 378.53) |
| Sub 2-51 | m/z = 375.19(C₂₈H₁₇D₄N = 375.51) | Sub 2-52 | m/z = 380.22(C₂₈H₁₂D₉N = 380.54) |
| Sub 2-53 | m/z = 553.19(C₄₀H₂₇NS = 553.72) | Sub 2-54 | m/z = 345.15(C₂₆H₁₉N = 345.45) |
| Sub 2-55 | m/z = 377.21(C₂₈H₂₇N = 377.53) | Sub 2-56 | m/z = 447.20(C₃₄H₂₅N = 447.58) |
| Sub 2-57 | m/z = 435.16(C₃₂H₂₁NO = 435.53) | Sub 2-58 | m/z = 451.14(C₃₂H₂₁NS = 451.59) |
| Sub 2-59 | m/z = 461.21(C₃₅H₂₇N = 461.61) | Sub 2-60 | m/z = 549.21(C₄₁H₂₇NO = 549.67) |
| Sub 2-61 | m/z = 533.21(C₄₁H₂₇N = 533.67) | Sub 2-62 | m/z = 389.21(C₂₉H₂₇N = 389.54) |
| Sub 2-63 | m/z = 487.23(C₃₇H₂₉N = 487.65) | Sub 2-64 | m/z = 461.18(C₃₄H₂₃NO = 461.56) |
| Sub 2-65 | m/z = 429.25(C₃₂H₃₁N = 429.61) | Sub 2-66 | m/z = 535.23(C₄₁H₂₉N = 535.69) |
| Sub 2-67 | m/z = 535.23(C₄₁H₂₉N = 535.69) | Sub 2-68 | m/z = 535.23(C₄₁H₂₉N = 535.69) |
| Sub 2-69 | m/z = 535.23(C₄₁H₂₉N = 535.69) | Sub 2-70 | m/z = 511.19(C₃₈H₂₅NO = 511.62) |
| Sub 2-71 | m/z = 461.18(C₃₄H₂₃NO = 461.56) | Sub 2-72 | m/z = 401.12(C₂₈H₁₉NS = 401.53) |
| Sub 2-73 | m/z = 551.19(C₄₀H₂₅NO₂ = 551.65) | Sub 2-74 | m/z = 371.17(C₂₈H₂₁N = 371.48) |
| Sub 2-75 | m/z = 451.19(C₃₃H₂₅NO = 451.57) | Sub 2-76 | m/z = 321.15(C₂₄H₁₉N = 321.42) |
| Sub 2-77 | m/z = 389.16(C₂₈H₂₀FN = 389.47) | Sub 2-78 | m/z = 325.15(C₂₃H₁₉NO = 325.41) |
| Sub 2-79 | m/z = 320.13(C₂₃H₁₆N₂ = 320.40) | Sub 2-80 | m/z = 624.26(C₄₇H₃₂N₂ = 624.79) |
| Sub 2-81 | m/z = 641.22(C₄₇H₃₁NS = 641.83) | Sub 2-82 | m/z = 425.21(C₃₂H₂₇N = 425.58) |
| Sub 2-83 | m/z = 563.26(C₄₃H₃₃N = 563.74) | Sub 2-84 | m/z = 611.26(C₄₇H₃₃N = 611.79) |
| Sub 2-85 | m/z = 411.20(C₃₁H₂₅N = 411.55) | Sub 2-86 | m/z = 487.23(C₃₇H₂₉N = 487.65) |
| Sub 2-87 | m/z = 435.16(C₃₂H₂₁NO = 435.53) | Sub 2-88 | m/z = 421.18(C₃₂H₂₃N = 421.54) |
| Sub 2-89 | m/z = 391.29(C₂₈HD₂₀N = 391.61) | Sub 2-90 | m/z = 471.20(C₃₆H₂₅N = 471.60) |
| Sub 2-91 | m/z = 371.17(C₂₈H₂₁N = 371.48) | Sub 2-92 | m/z = 351.20(C₂₆H₂₅N = 351.49) |
| Sub 2-93 | m/z = 429.25(C₃₂H₃₁N = 429.61) | Sub 2-94 | m/z = 351.20(C₂₆H₂₅N = 351.49) |
| Sub 2-95 | m/z = 351.20(C₂₆H₂₅N = 351.49) | Sub 2-96 | m/z = 579.29(C₄₄H₃₇N = 579.79) |
| Sub 2-97 | m/z = 451.23(C₃₄H₂₉N = 451.61) | Sub 2-98 | m/z = 427.19(C₃₁H₂₅NO = 427.55) |
| Sub 2-99 | m/z = 453.25(C₃₄H₃₁N = 453.63) | Sub 2-100 | m/z = 553.22(C₄₀H₃₁NSi = 553.78) |
| Sub 2-101 | m/z = 477.17(C₃₄H₂₃NO₂ = 477.56) | Sub 2-102 | m/z = 417.12(C₂₈H₁₉NOS = 417.53) |
| Sub 2-103 | m/z = 295.14(C₂₂H₁₇N = 295.39) | Sub 2-104 | m/z = 551.21(C₄₀H₂₉NSi = 551.76) |
| Sub 2-105 | m/z = 487.23(C₃₇H₂₉N = 487.65) | Sub 2-106 | m/z = 535.23(C₄₁H₂₉N = 535.69) |
| Sub 2-107 | m/z = 461.18(C₃₄H₂₃NO = 461.56) | Sub 2-108 | m/z = 487.23(C₃₇H₂₉N = 487.65) |
| Sub 2-109 | m/z = 411.20(C₃₁H₂₅N = 411.55) | Sub 2-110 | m/z = 295.14(C₂₂H₁₇N = 295.39) |

### Synthesis Example of Final Compound

### 1. Synthesis Example of P-13

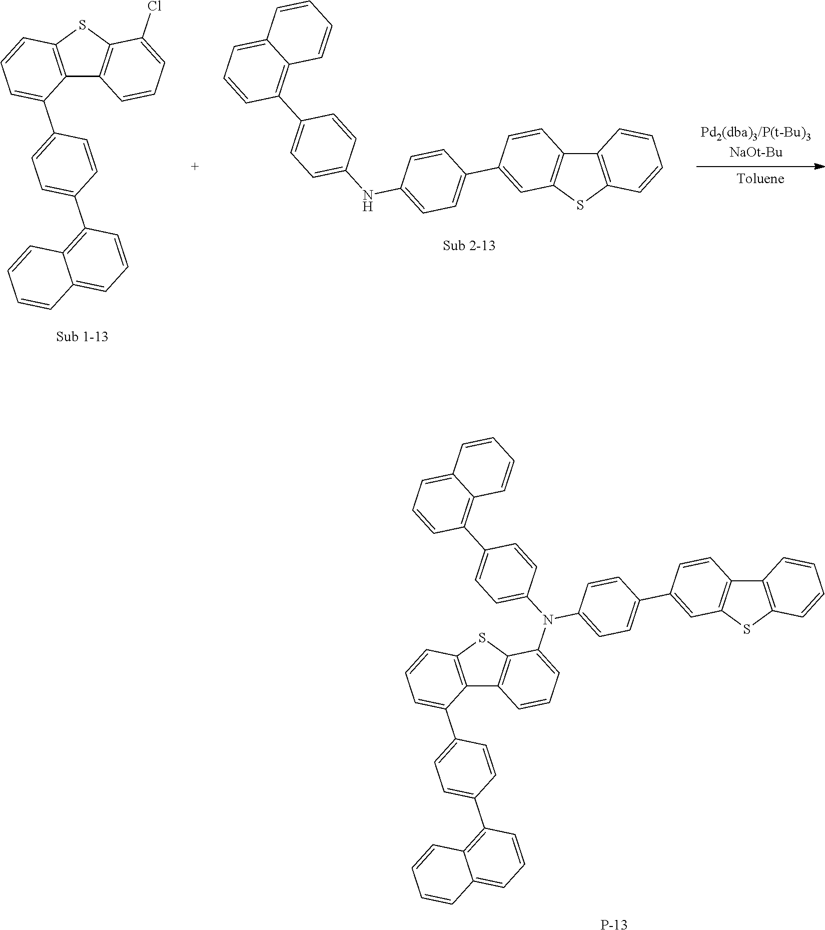

After dissolving Sub 1-13 (20.0 g, 47.5 mmol) in toluene (238 mL), Sub 2-13 (21.9 g, 47.5 mmol), Pd₂(dba)₃ (1.3 g, 1.4 mmol), P(t-Bu)₃ (0.6 g, 2.6 mmol), and NaOt-Bu (9.1 g, 95.0 mmol) were added thereto and the mixture was stirred at 120° C. When the reaction was completed, the reaction product was extracted with CH₂Cl₂ and water. Then, an organic layer was dried over MgSO₄ and concentrated. Then, the concentrate was separated by a silica gel column and recrystallized to obtain 29.0 g (yield: 70.9%) of the product.

### 2. Synthesis Example of P-21

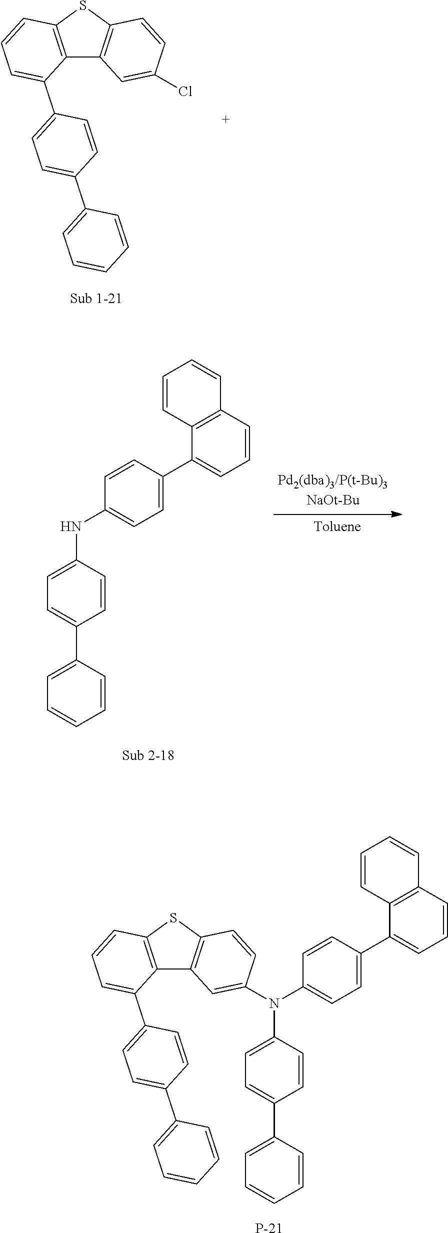

Using Sub 1-21 (20.0 g, 53.9 mmol), Sub 2-18 (19.4 g, 53.9 mmol), Pd₂(dba)₃ (1.5 g, 1.6 mmol), P(t-Bu)₃ (0.7 g, 3.2 mmol), NaOt-Bu (10.4 g, 107.8 mmol), and toluene (270 mL), the reaction was carried out in the same manner as the synthesis method of P-1, and then purified to obtain 27.5 g (yield: 72.2%) of product.

### 3. Synthesis Example of P-34

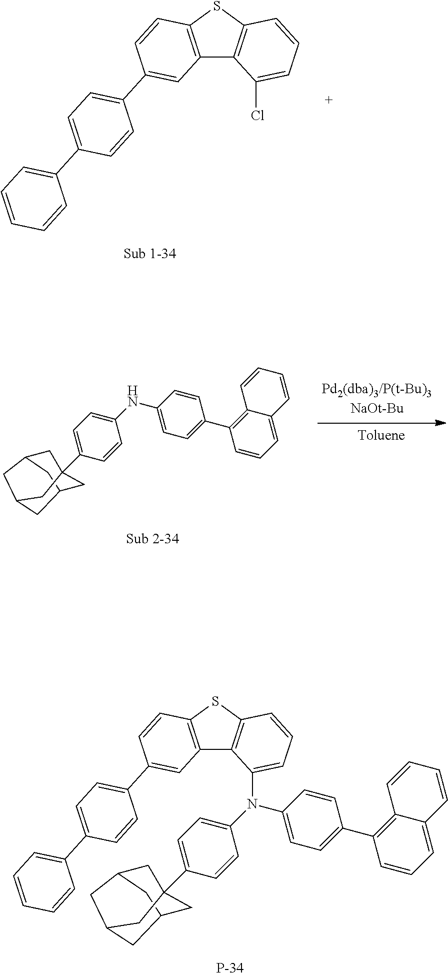

Using Sub 1-34 (20.0 g, 53.9 mmol), Sub 2-34 (22.4 g, 53.9 mmol), Pd₂(dba)₃ (1.5 g, 1.6 mmol), P(t-Bu)₃ (0.7 g, 3.2 mmol), NaOt-Bu (10.4 g, 107.8 mmol), and toluene (270 mL), the reaction was carried out in the same manner as the synthesis method of P-1, and then purified to obtain 29.5 g (yield: 71.7%) of product.

### 4. Synthesis Example of P-44

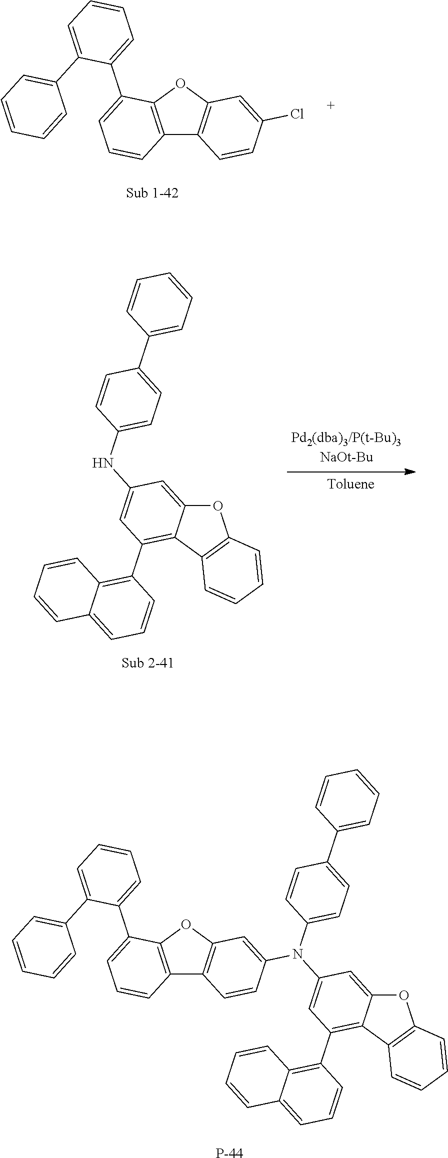

Using Sub 1-42 (20.0 g, 56.4 mmol), Sub 2-41 (25.1 g, 56.4 mmol), Pd₂(dba)₃ (1.6 g, 1.7 mmol), P(t-Bu)₃ (0.7 g, 3.4 mmol), NaOt-Bu (10.8 g, 112.7 mmol), and toluene (282 mL), the reaction was carried out in the same manner as the synthesis method of P-1, and then purified to obtain 30.8 g (yield: 70.0%) of product.

### 5. Synthesis Example of P-51

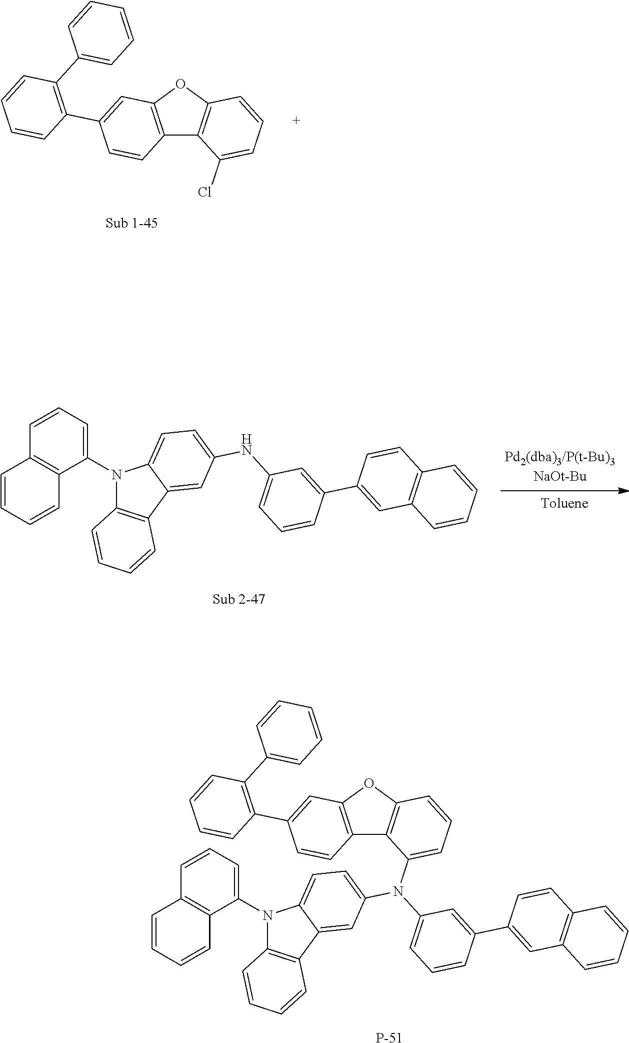

Using Sub 1-45 (20.0 g, 56.4 mmol), Sub 2-47 (27.8 g, 56.4 mmol), Pd₂(dba)₃ (1.6 g, 1.7 mmol), P(t-Bu)₃ (0.7 g, 3.4 mmol), NaOt-Bu (10.8 g, 112.7 mmol), and toluene (282 mL), the reaction was carried out in the same manner as the synthesis method of P-1, and then purified to obtain 32.6 g (yield: 69.7%) of product.

### 6. Synthesis Example of P-68

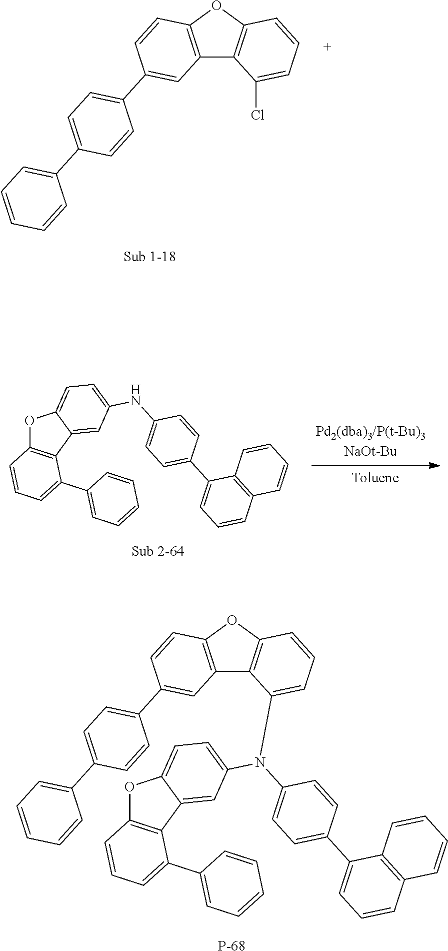

Using Sub 1-18 (20.0 g, 56.4 mmol), Sub 2-64 (25.1 g, 56.4 mmol), Pd₂(dba)₃ (1.6 g, 1.7 mmol), P(t-Bu)₃ (0.7 g, 3.4 mmol), NaOt-Bu (10.8 g, 112.7 mmol) toluene (282 mL), the reaction was carried out in the same manner as the synthesis method of P-1, and then purified to obtain 30.6 g (yield: 72.6%) of product.

### 7. Synthesis Example of P-77

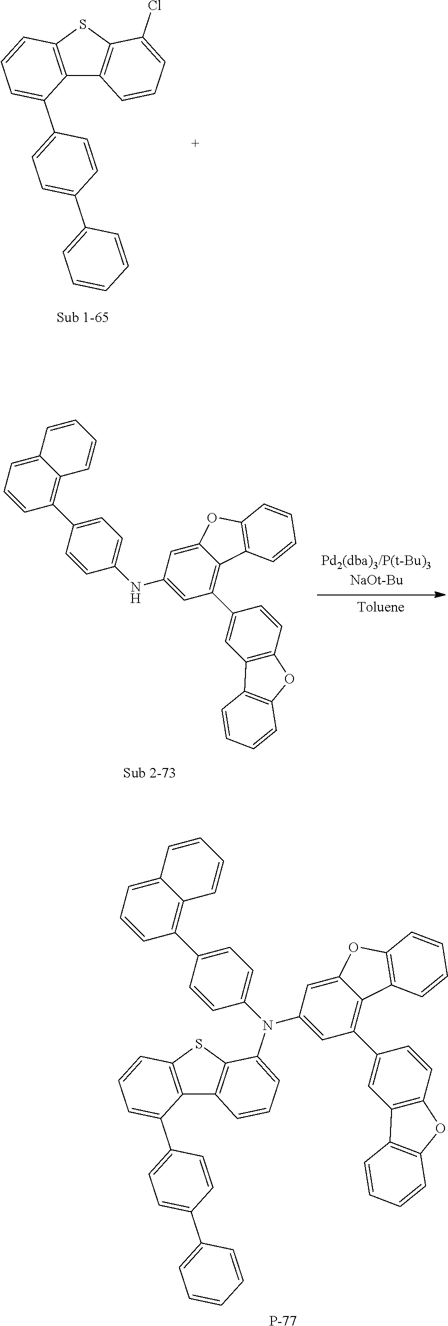

Using Sub 1-65 (20.0 g, 53.9 mmol), Sub 2-73 (28.7 g, 53.9 mmol), Pd₂(dba)₃ (1.5 g, 1.6 mmol), P(t-Bu)₃ (0.7 g, 3.2 mmol), NaOt-Bu (10.4 g, 107.8 mmol), and toluene (270 mL), the reaction was carried out in the same manner as the synthesis method of P-1, and then purified to obtain 33.2 g (yield: 69.5%) of product.

### 8. Synthesis Example of P-86

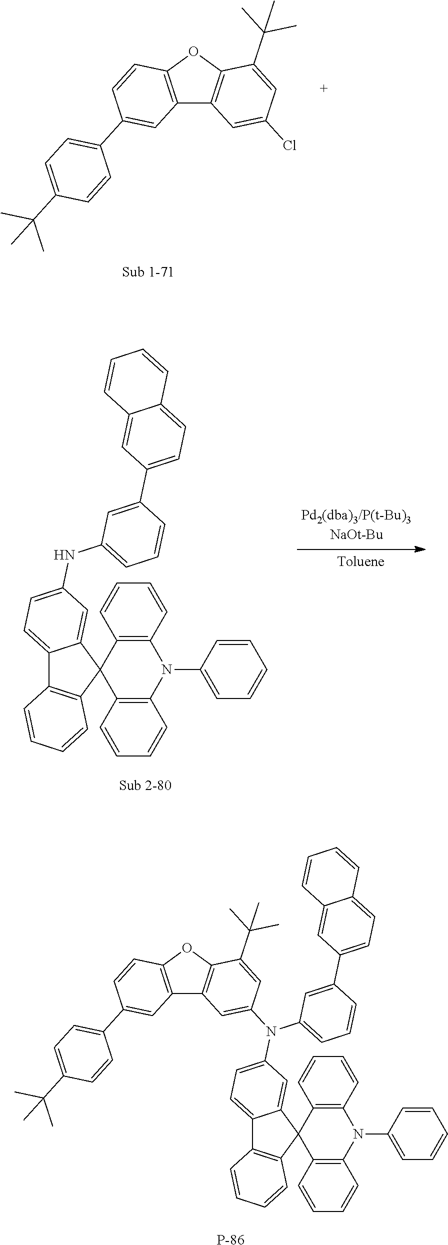

Using Sub 1-71 (20.0 g, 51.2 mmol), Sub 2-80 (30.9 g, 51.2 mmol), Pd₂(dba)₃ (1.4 g, 1.5 mmol), P(t-Bu)₃ (0.6 g, 3.1 mmol), NaOt-Bu (9.8 g, 102.3 mmol), and toluene (256 mL), the reaction was carried out in the same manner as the synthesis method of P-1, and then purified to obtain 36.3 g (yield 72.4%) of product.

### 9. Synthesis Example of P-92

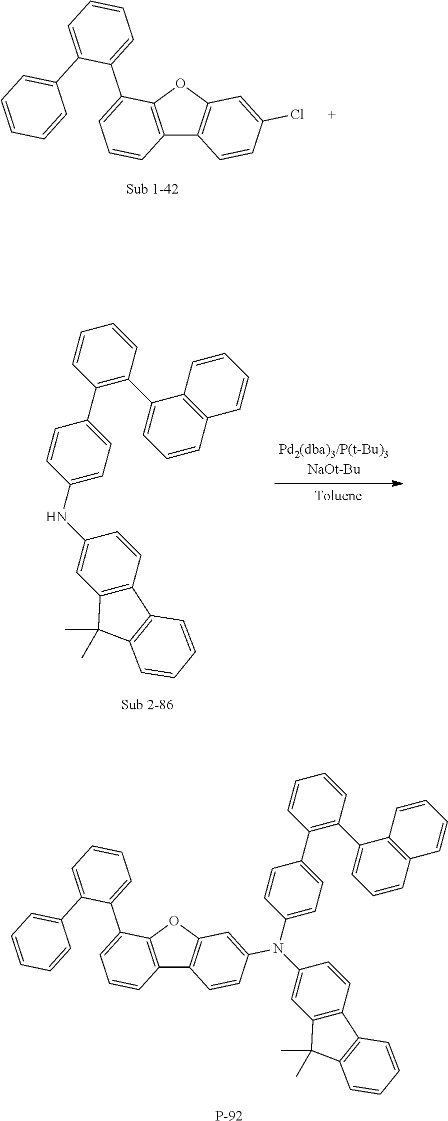

Using Sub 1-42 (20.0 g, 56.4 mmol), Sub 2-86 (26.6 g, 56.4 mmol), Pd₂(dba)₃ (1.6 g, 1.7 mmol), P (t-Bu) 3 (0.7 g, 3.4 mmol), NaOt-Bu (10.8 g, 112.7 mmol) toluene (282 mL), the reaction was carried out in the same manner as the synthesis method of P-1, and then purified to obtain 32.3 g (yield: 71.2%) of product.

### 10. Synthesis Example of P-101

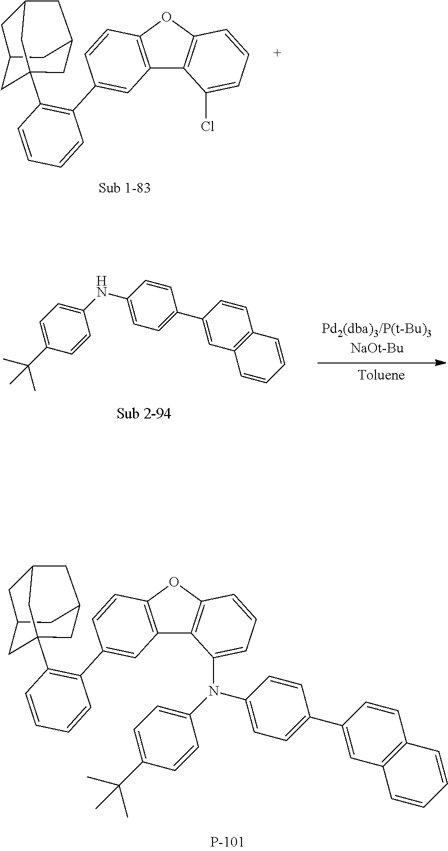 [SMILES: Clc1cccc2oc3ccc(-c4ccccc4C45CC6CC(CC(C6)C4)C5)cc3c12 ; CC(C)(C)c1ccc(Nc2ccc(-c3ccc4ccccc4c3)cc2)cc1 ; CC(C)(C)c1ccc(N(c2ccc(-c3ccc4ccccc4c3)cc2)c2cccc3oc4ccc(-c5ccccc5C56CC7CC(CC(C7)C5)C6)cc4c23)cc1]

Using Sub 1-83 (20.0 g, 48.4 mmol), Sub 2-94 (16.4 g, 48.4 mmol), Pd₂(dba)₃ (1.3 g, 1.5 mmol), P(t-Bu)₃ (0.6 g, 2.9 mmol), NaOt-Bu (9.3 g, 96.9 mmol), and toluene (242 mL), the reaction was carried out in the same manner as the synthesis method of P-1, and then purified to obtain 25.3 g (yield: 71.8%) of product.

FD-MS values of the compounds P-1 to P-118 of the present invention synthesized by the above synthesis method are shown in Table 3 below.

TABLE 3

| Compound | FD-MS | Compound | FD-MS |
|---|---|---|---|
| P-1 | m/z = 689.27(C₅₂H₃₅NO = 689.86) | P-2 | m/z = 613.24(C₄₆H₃₁NO = 613.76) |
| P-3 | m/z = 553.19(C₄₀H₂₇NS = 553.72) | P-4 | m/z = 705.25(C₅₂H₃₅NS = 705.92) |
| P-5 | m/z = 603.20(C₄₄H₂₉NS = 603.78) | P-6 | m/z = 603.20(C₄₄H₂₉NS = 603.78) |
| P-7 | m/z = 587.22(C₄₄H₂₉NO = 587.72) | P-8 | m/z = 729.30(C₅₅H₃₉NO = 729.92) |
| P-9 | m/z = 689.27(C₅₂H₃₅NO = 689.86) | P-10 | m/z = 765.30(C₅₈H₃₉NO = 765.96) |
| P-11 | m/z = 753.25(C₅₆H₃₅NS = 753.96) | P-12 | m/z = 831.30(C₆₂H₄₁NS = 832.08) |
| P-13 | m/z = 861.25(C₆₂H₃₉NS₂ = 862.12) | P-14 | m/z = 831.30(C₆₂H₄₁NS = 832.08) |
| P-15 | m/z = 815.32(C₆₂H₄₁NO = 816.02) | P-16 | m/z = 815.32(C₆₂H₄₁NO = 816.02) |
| P-17 | m/z = 765.30(C₅₈H₃₉NO = 765.96) | P-18 | m/z = 689.27(C₅₂H₃₅NO = 689.86) |
| P-19 | m/z = 705.25(C₅₂H₃₅NS = 705.92) | P-20 | m/z = 705.25(C₅₂H₃₅NS = 705.92) |
| P-21 | m/z = 705.25(C₅₂H₃₅NS = 705.92) | P-22 | m/z = 781.28(C₅₈H₃₉NS = 782.02) |
| P-23 | m/z = 739.29(C₅₆H₃₇NO = 739.92) | P-24 | m/z = 779.28(C₅₈H₃₇NO₂ = 779.94) |
| P-25 | m/z = 689.27(C₅₂H₃₅NO = 689.86) | P-26 | m/z = 765.30(C₅₈H₃₉NO = 765.96) |
| P-27 | m/z = 885.27(C₆₄H₃₉NO₂S = 886.08) | P-28 | m/z = 795.30(C₅₉H₄₁NS = 796.04) |
| P-29 | m/z = 719.23(C₅₂H₃₃NOS = 719.9) | P-30 | m/z = 887.27(C₆₄H₄₁NS₂ = 888.16) |
| P-31 | m/z = 779.28(C₅₈H₃₇NO₂ = 779.94) | P-32 | m/z = 815.32(C₆₂H₄₁NO = 816.02) |
| P-33 | m/z = 749.31(C₅₅H₃₅D₄NS = 750.01) | P-34 | m/z = 763.33(C₅₆H₄₅NS = 764.04) |
| P-35 | m/z = 779.28(C₅₈H₃₇NO₂ = 779.94) | P-36 | m/z = 739.29(C₅₆H₃₇NO = 739.92) |
| P-37 | m/z = 723.30(C₅₃H₄₁NS = 723.98) | P-38 | m/z = 663.26(C₅₀H₃₃NO = 663.82) |
| P-39 | m/z = 685.28(C₅₀H₃₉NS = 685.93) | P-40 | m/z = 745.28(C₅₅H₃₉NS = 745.98) |
| P-41 | m/z = 755.26(C₅₆H₃₇NS = 755.98) | P-42 | m/z = 735.21(C₅₂H₃₃NS₂ = 735.96) |
| P-43 | m/z = 751.33(C₅₅H₄₅NS = 752.03) | P-44 | m/z = 779.28(C₅₈H₃₇NO₂ = 779.94) |
| P-45 | m/z = 739.29(C₅₆H₃₇NO = 739.92) | P-46 | m/z = 613.24(C₄₆H₃₁NO = 613.76) |
| P-47 | m/z = 889.33(C₆₈H₄₃NO = 890.10) | P-48 | m/z = 821.31(C₆₁H₄₃NS = 822.08) |
| P-49 | m/z = 828.31(C₆₂H₄₀N₂O = 829.02) | P-50 | m/z = 685.28(C₅₀H₃₉NS = 685.93) |
| P-51 | m/z = 828.31(C₆₂H₄₀N₂O = 829.02) | P-52 | m/z = 845.28(C₆₂H₃₉NOS = 846.06) |
| P-53 | m/z = 669.30(C₅₀H₃₉NO = 669.87) | P-54 | m/z = 712.29(C₅₂H₂₈D₇NS = 712.96) |
| P-55 | m/z = 750.26(C₅₃H₃₀D₄N₂OS = 750.95) | P-56 | m/z = 714.31(C₅₂H₂₆D₉NS = 714.97) |
| P-57 | m/z = 947.32(C₇₀H₄₅NOS = 948.20) | P-58 | m/z = 679.23(C₅₀H₃₃NS = 679.88) |
| P-59 | m/z = 793.37(C₅₈H₅₁NS = 794.11) | P-60 | m/z = 781.28(C₅₈H₃₉NS = 782.02) |
| P-61 | m/z = 829.30(C₆₂H₃₉NO₂ = 830.00) | P-62 | m/z = 859.25(C₆₂H₃₇NO₂S = 860.04) |
| P-63 | m/z = 869.33(C₆₅H₄₃NO₂ = 870.06) | P-64 | m/z = 983.32(C₇₃H₄₅NOS = 984.23) |
| P-65 | m/z = 867.30(C₆₅H₄₁NS = 868.11) | P-66 | m/z = 765.40(C₅₇H₅₁NO = 766.04) |
| P-67 | m/z = 821.31(C₆₁H₄₃NS = 822.08) | P-68 | m/z = 779.28(C₅₈H₃₇NO₂ = 779.94) |
| P-69 | m/z = 781.37(C₅₇H₅₁NS = 782.10) | P-70 | m/z = 853.33(C₆₅H₄₃NO = 854.07) |
| P-71 | m/z = 853.33(C₆₅H₄₃NO = 854.07) | P-72 | m/z = 945.34(C₇₁H₄₇NS = 946.22) |
| P-73 | m/z = 869.31(C₆₅H₄₃NS = 870.13) | P-74 | m/z = 845.28(C₆₂H₃₉NOS = 846.06) |
| P-75 | m/z = 855.31(C₆₄H₄₁NO₂ = 856.04) | P-76 | m/z = 719.23(C₅₂H₃₃NOS = 719.90) |
| P-77 | m/z = 885.27(C₆₄H₃₉NO₂S = 886.08) | P-78 | m/z = 855.31(C₆₄H₄₁NO₂ = 856.04) |
| P-79 | m/z = 874.30(C₆₃H₄₂N₂OS = 875.10) | P-80 | m/z = 655.23(C₄₈H₃₃NS = 655.86) |
| P-81 | m/z = 773.26(C₅₆H₃₆FNS = 773.97) | P-82 | m/z = 747.35(C₅₆H₄₅NO = 747.98) |
| P-83 | m/z = 643.25(C₄₇H₃₃NO₂ = 643.79) | P-84 | m/z = 638.24(C₄₇H₃₀N₂O = 638.77) |
| P-85 | m/z = 723.30(C₅₃H₄₁NS = 723.98) | P-86 | m/z = 978.45(C₇₃H₅₈N₂O = 979.28) |
| P-87 | m/z = 975.30(C₇₁H₄₅NS₂ = 976.27) | P-88 | m/z = 759.30(C₅₆H₄₁NS = 760.01) |
| P-89 | m/z = 897.34(C₆₇H₄₇NS = 898.18) | P-90 | m/z = 945.34(C₇₁H₄₇NS = 946.22) |
| P-91 | m/z = 729.30(C₅₅H₃₉NO = 729.92) | P-92 | m/z = 805.33(C₆₁H₄₃NO = 806.02) |
| P-93 | m/z = 753.27(C₅₆H₃₅NO₂ = 753.90) | P-94 | m/z = 739.29(C₅₆H₃₇NO = 739.92) |
| P-95 | m/z = 715.47(C₅₀H₉D₃₀NS = 716.11) | P-96 | m/z = 805.28(C₆₀H₃₉NS = 806.04) |
| P-97 | m/z = 705.25(C₅₂H₃₅NS = 705.92) | P-98 | m/z = 703.33(C₅₁H₄₅NS = 703.99) |
| P-99 | m/z = 743.36(C₅₄H₄₉NS = 744.05) | P-100 | m/z = 741.34(C₅₄H₄₇NS = 742.04) |
| P-101 | m/z = 727.38(C₅₄H₄₉NO = 727.99) | P-102 | m/z = 669.30(C₅₀H₃₉NO = 669.87) |
| P-103 | m/z = 687.35(C₅₁H₄₅NO = 687.93) | P-104 | m/z = 727.38(C₅₄H₄₉NO = 727.99) |
| P-105 | m/z = 913.37(C₆₈H₅₁NS = 914.22) | P-106 | m/z = 845.37(C₆₄H₄₇NO = 846.09) |
| P-107 | m/z = 789.31(C₅₇H₄₃NOS = 790.04) | P-108 | m/z = 772.35(C₅₇H₄₄N₂O = 772.99) |
| P-109 | m/z = 871.33(C₆₄H₄₅NOSi = 872.16) | P-110 | m/z = 867.32(C₆₂H₄₅NO₂S = 868.11) |
| P-111 | m/z = 869.33(C₆₂H₄₇NO₂S = 870.12) | P-112 | m/z = 729.34(C₅₃H₄₇NS = 730.03) |
| P-113 | m/z = 869.31(C₆₄H₄₃NOSi = 870.14) | P-114 | m/z = 885.29(C₆₄H₄₃NSSi = 886.20) |
| P-115 | m/z = 821.31(C₆₁H₄₃NS = 822.08) | P-116 | m/z = 853.33(C₆₅H₄₃NO = 854.07) |
| P-117 | m/z = 779.28(C₅₈H₃₇NO₂ = 779.94) | P-118 | m/z = 821.31(C₆₁H₄₃NS = 822.08) |

### Fabrication and Evaluation of Organic Electric Element

### [Test Example 1] Red Organic Electroluminescent Element (Emission-Auxiliary Layer)

After vacuum-depositing 4,4′,4″-tris[2-naphthyl(phenyl)amino]triphenylamine (hereinafter abbreviated as “2-TNATA”) on an ITO layer (anode) formed on a glass substrate to form a hole injection layer of 60 nm thickness, a hole transport layer of 60 nm thickness was formed by vacuum-depositing N,N′-bis(1-naphthalenyl)-N,N′-bis-phenyl-(1,1′-biphenyl)-4,4′-diamine (hereinafter abbreviated as “NPB”) on the hole injection layer.

Subsequently, an emission-auxiliary layer of 20 nm thickness was formed by vacuum-depositing the compound P-1 of the present invention on the hole transport layer and 4,4′-N,N′-dicarbazole-biphenyl (hereinafter abbreviated as “CBP”) as a host material and bis-(1-phenyl isoquinolyl)iridium(III)acetylacetonate (hereinafter abbreviated as “(piq)₂Ir(acac)”) as a dopant material in a weight ratio of 95:5 were deposited on the emission-auxiliary layer to form a light emitting layer of 30 nm thickness.

Next, (1,1′-bisphenyl-4-olato)bis(2-methyl-8-quinolinolato)aluminum (hereinafter abbreviated as “BAlq”) was vacuum-deposited to form a hole blocking layer of 10 nm thickness on the light emitting layer, and tris-(8-hydroxyquinoline)aluminum (hereinafter abbreviated as “Alq3”) was vacuum-deposited to form a an electron transport layer of 40 nm thickness on the hole blocking layer. Thereafter, LiF was deposited to form an electron injection layer of 0.2 nm thickness on the electron transport layer, and then Al was deposited to form a cathode of 150 nm thickness on the electron injection layer.

### [Test Example 2] to [Test Example 23]

The organic electroluminescent elements were fabricated in the same manner as described in Example 1 except that the compounds of the present invention described in the following Table 4, instead of compound P-1 of the present invention, were used as material of an emission-auxiliary layer.

### [Comparative Example 1] to [Comparative Example 3]

The organic electroluminescent elements were fabricated in the same manner as described in Example 1 except that one of Comparative Compounds A to C, instead of compound P-1 of the present invention, was used as material of an emission-auxiliary layer.

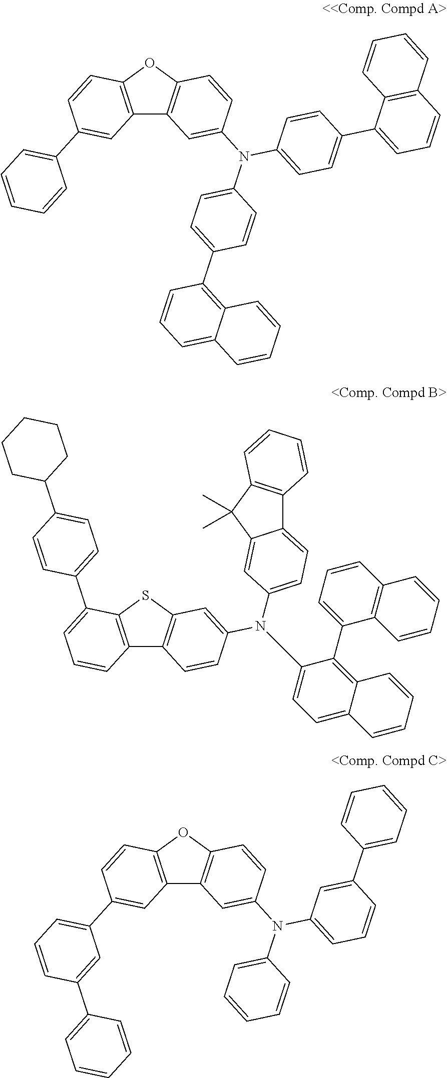 [SMILES: c1ccc(-c2ccc3oc4ccc(N(c5ccc(-c6cccc7ccccc67)cc5)c5ccc(-c6cccc7ccccc67)cc5)cc4c3c2)cc1 ; CC1(C)c2ccccc2-c2ccc(N(c3ccc4c(c3)sc3c(-c5ccc(C6CCCCC6)cc5)cccc34)c3ccc4ccccc4c3-c3cccc4ccccc34)cc21 ; c1ccc(-c2cccc(-c3ccc4oc5ccc(N(c6ccccc6)c6cccc(-c7ccccc7)c6)cc5c4c3)c2)cc1]

A forward bias DC voltage was applied to the electroluminescent elements manufactured in Test Examples and Comparative Examples, and electroluminescence (EL) characteristics were measured with Photo Research's PR-650 and lifetime (T95) was measured with a lifetime measuring device manufactured by Mc Science company at 2500 cd/m² standard luminance. The measurement results are shown in Table 4 below.

TABLE 4

| | | | Current | | | |
|---|---|---|---|---|---|---|
| | | | Density | Brightness | Efficiency | Lifetime |
| | Compound | Voltage (V) | (mA/cm²) | (cd/m²) | (cd/A) | T(95) |
| comp.Ex(1) | Comp. compd A | 5.2 | 2.9 | 2500.0 | 17.6 | 87.6 |
| comp.Ex(2) | Comp. compd B | 5.3 | 2.9 | 2500.0 | 18.2 | 86.3 |
| comp.Ex(3) | Comp. compd C | 5.3 | 2.8 | 2500.0 | 16.6 | 88.4 |
| Test Ex.(1) | P-1 | 4.9 | 11.3 | 2500.0 | 22.1 | 114.7 |
| Test Ex.(2) | P-13 | 5.0 | 12.1 | 2500.0 | 20.6 | 100.3 |
| Test Ex.(3) | P-18 | 4.9 | 10.9 | 2500.0 | 23.0 | 115.6 |
| Test Ex.(4) | P-21 | 4.8 | 10.1 | 2500.0 | 24.8 | 117.7 |
| Test Ex.(5) | P-23 | 5.0 | 10.8 | 2500.0 | 23.2 | 107.9 |
| Test Ex.(6) | P-24 | 5.0 | 11.2 | 2500.0 | 22.3 | 106.0 |
| Test Ex.(7) | P-34 | 4.9 | 10.6 | 2500.0 | 23.6 | 110.8 |
| Test Ex.(8) | P-42 | 5.0 | 11.1 | 2500.0 | 22.6 | 101.3 |
| Test Ex.(9) | P-43 | 5.0 | 11.9 | 2500.0 | 21.0 | 101.2 |
| Test Ex.(10) | P-44 | 5.0 | 11.4 | 2500.0 | 21.9 | 103.9 |
| Test Ex.(11) | P-46 | 5.0 | 11.0 | 2500.0 | 22.8 | 104.3 |
| Test Ex.(12) | P-51 | 5.0 | 10.9 | 2500.0 | 23.0 | 112.2 |
| Test Ex.(13) | P-53 | 4.8 | 10.5 | 2500.0 | 23.9 | 118.5 |
| Test Ex.(14) | P-57 | 5.0 | 10.9 | 2500.0 | 22.9 | 105.7 |
| Test Ex.(15) | P-58 | 5.0 | 11.2 | 2500.0 | 22.4 | 102.6 |
| Test Ex.(16) | P-68 | 4.9 | 10.8 | 2500.0 | 23.1 | 116.8 |
| Test Ex.(17) | P-69 | 4.8 | 10.2 | 2500.0 | 24.4 | 109.1 |
| Test Ex.(18) | P-76 | 5.0 | 11.6 | 2500.0 | 21.5 | 103.4 |
| Test Ex.(19) | P-77 | 5.0 | 11.8 | 2500.0 | 21.1 | 101.7 |
| Test Ex.(20) | P-92 | 5.0 | 11.8 | 2500.0 | 21.2 | 102.8 |
| Test Ex.(21) | P-99 | 4.8 | 10.4 | 2500.0 | 24.1 | 108.5 |
| Test Ex.(22) | P-101 | 4.9 | 11.0 | 2500.0 | 22.7 | 113.1 |
| Test Ex.(23) | P-115 | 4.9 | 10.6 | 2500.0 | 23.5 | 111.6 |

As can be seen from the results in Table 4 above, when a red organic electroluminescent element is manufactured using the material for an organic electroluminescent element of the present invention as material for a light-emitting auxiliary layer, the driving voltage, the luminous efficiency and lifespan of the organic electroluminescence element can be improved, compared to the comparative examples using one of Comparative Compounds A to C which have a similar basic structure to the compounds of the present invention.

Comparative Compound A is an amine compound similar to the compound of the present invention, but differs from the present invention in that the substituents corresponding to R1 to R5 of the present invention are all hydrogen, except for the case where all substituents corresponding to R¹ to R⁵ are hydrogen.

Comparative compound B is an amine compound similar to the compound of the present invention, but differs from the present invention, of which L is a non-fused arylene, in that the linker corresponding to L of the present invention is naphthalene, which is a condensed arylene.

Comparative compound C is also an amine compound similar to the compound of the present invention, but differs in that the substituent corresponding to the naphthalene moiety bonded to the linker L of the present invention is simple phenyl.

Due to these differences, it is assumed that the compound of the present invention has more suitable physical properties than comparative compounds when used as a light-emitting auxiliary layer material.

In the compound of the present invention, one of the substituents substituted on the amine contains a non-fused arylene group-naphthyl moiety being a linker. It appears that these substituents affect the formation of an energy level suitable for the red light-emitting auxiliary layer.

In the case of comparative compound B, in which a naphthyl moiety is substituted on the condensed arylene group linker, the HOMO-LUMO band gap is reduced compared to the compound of the present invention, and in the case of comparative compound C, in which phenyl is substituted on the phenyl linker, a smaller HOMO electron cloud is formed than the compound of the present invention. Therefore, it appears that the compound of the present invention has superior hole injection and hole transport characteristics than Comparative Compound B and C.

In particular, compared to Comparative Compound A, the compound of the present invention has a substituent other than hydrogen bonded to phenyl, resulting in increased steric hindrance compared to Comparative Compound A.

Accordingly, the effect of suppressing π-π stacking between molecules occurs, and the Tg value decreases although the planarity of the molecules decreases during material deposition. Therefore, it becomes possible to manufacture elements even at relatively low temperatures.

In addition, as the distance between molecules increases due to steric hindrance, the crystallinity of the thin film can be lowered, that is, an amorphous state can be created. As a result, it appears that hole mobility is improved and the stability of the compound itself is increased, which the overall performance of the element is improved.

Among the compounds of the present invention, the characteristics of the element were significantly excellent in case of the compound in which an amine group is substituted at the 1st or 2nd position of dibenzofuran or dibenzothiophene, and at the same time, a phenyl group is bonded to the 8th or 9th position of dibenzofuran or dibenzothiophene, wherein the phenyl group is substituted with a substituent other than hydrogen. This appears to be because the steric hindrance effect is maximized more than when a substituent is introduced at a different position.

These results suggest that the hole characteristics, light efficiency characteristics, energy level, hole injection and mobility characteristics, charge balance of holes and electrons, volume density, and intermolecular distance are affected depending on the substitution position of the substituent and the type of the substituent even if the basic structure is a similar compound, and the properties of the element may also vary due to differences in the properties of these complex compounds.

In addition, in the case of a light-emitting auxiliary layer, it is necessary to understand the interrelationship between the hole transport layer and the light-emitting layer (host). Therefore, it would be very difficult for a person skilled in the art to infer the characteristics exhibited by the light emitting auxiliary layer using the compound of the present invention even if a similar core is used.

In addition, in the above-described element fabrication evaluation results, the characteristics of the element were explained by applying the compound of the present invention only to the light-emitting auxiliary layer, but the compound of the present invention can be applied to the hole transport layer or applied to both the hole transport layer and the light-emitting auxiliary layer.

Although the exemplary embodiments of the present invention have been described for illustrative purposes, those skilled in the art to which the present invention pertains will be capable of various modifications without departing from the essential characteristics of the present invention. The scope of the present invention shall be construed based on the accompanying claims, and it shall be construed that all the technical arts included within the scope equivalent to the claims belong to the present invention.

What is claimed is:

1. A compound of Formula 1: 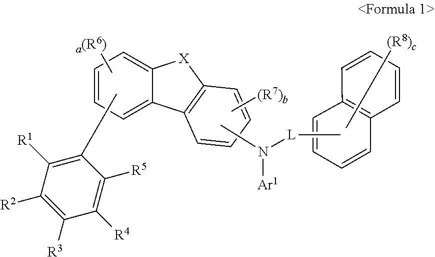
wherein:
X is O or S,
R¹ to R⁵ are each independently selected from the group consisting of hydrogen, deuterium, halogen, a cyano group, a nitro group, a C₆-C₆₀ aryl group, a fluorenyl group, a C₂-C₆o heterocyclic group containing at least one heteroatom of O, N, S, Si and P, a C₃-C₆o aliphatic ring group, a C₁-C₂₀ alkyl group, a C₂-C₂₀ alkenyl group, a C₂-C₂₀ alkynyl group, a C₁-C₂₀ alkoxyl group, and a C₆-C₂₀ aryloxy group, wherein adjacent groups may be bonded to each other to form a ring of a six-membered or more, and the case where R¹ to R⁵ are all hydrogen is excluded,
R⁶ to R⁸ are each independently selected from the group consisting of hydrogen, deuterium, halogen, a cyano group, a nitro group, a C₆-C₆₀ aryl group, a fluorenyl group, a C₂-C₆₀ heterocyclic group containing at least one heteroatom of O, N, S, Si and P, a C₃-C₆₀ aliphatic ring group, a C₁-C₂₀ alkyl group, a C₂-C₂₀ alkenyl group, a C₂-C₂₀ alkynyl group, a C₁-C₂₀ alkoxyl group, and a C₆-C₂₀ aryloxy group, wherein adjacent groups may be bonded to each other to form a ring,
a and b are each an integer of 0 to 3, c is an integer of 0 to 7, and where a, b, and c are each an integer of 2 or more, each of R⁶s, each of R⁷s, each of R⁸s are the same as or different from each other,
Ar¹ is selected from the group consisting of a C₆-C₆₀ aryl group, a fluorenyl group, a C₂-C₆₀ heterocyclic group containing at least one heteroatom of O, N, S, Si and P, and a C₃-C₆₀ aliphatic ring group,
L is selected from the group consisting of a non-fused C₆-C₆₀ arylene group, a fluorenylene group, a C₃-C₆₀ aliphatic ring group, and a C₂-C₆₀ heterocyclic group containing at least one heteroatom of O, N, S, Si and P, and
the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, and the ring formed by adjacent groups may be each optionally substituted with one or more substituents selected from the group consisting of deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group.

2. The compound of claim 1, wherein Formula 1 is represented by one of Formula 1-1 to Formula 1-8: 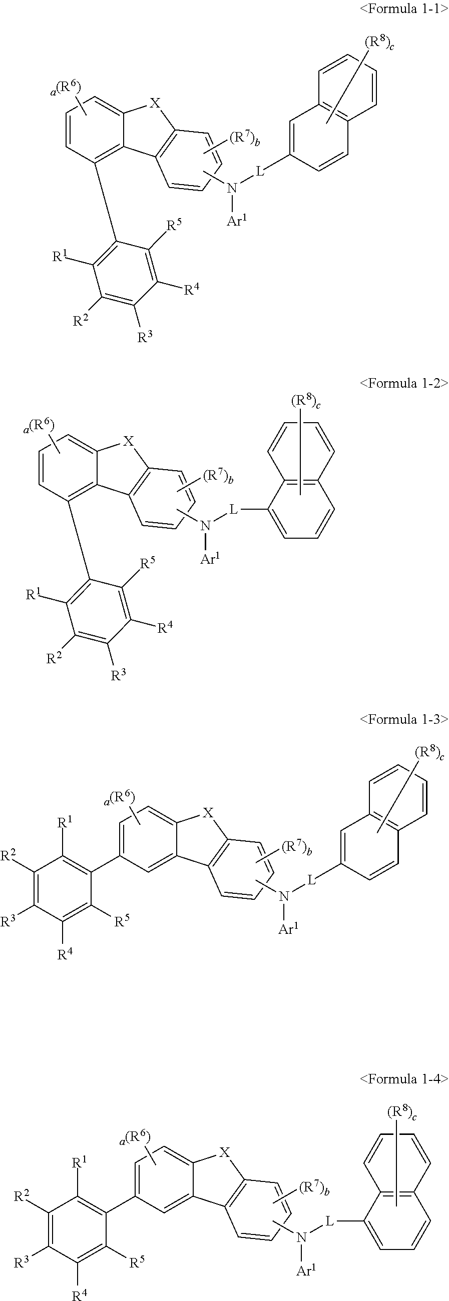 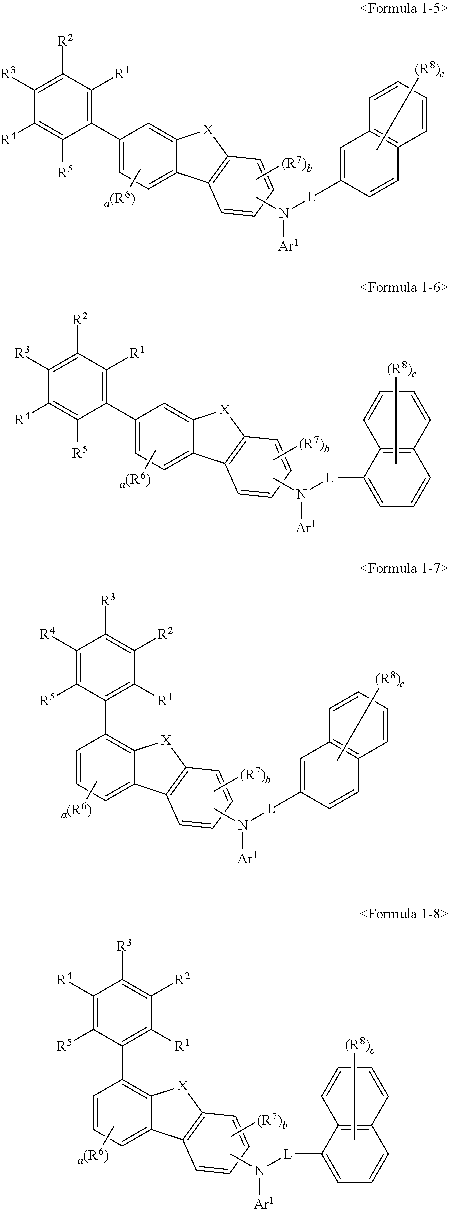
in Formula 1-1 to Formula 1-8, X, Ar¹, L, R¹ to R⁸, and a to c are the same as defined in claim 1.

3. The compound of claim 1, wherein L is a non-fused C₆-C₆₀ arylene group.

4. The compound of claim 1, wherein L is one of Formula L-1 to Formula L-3: 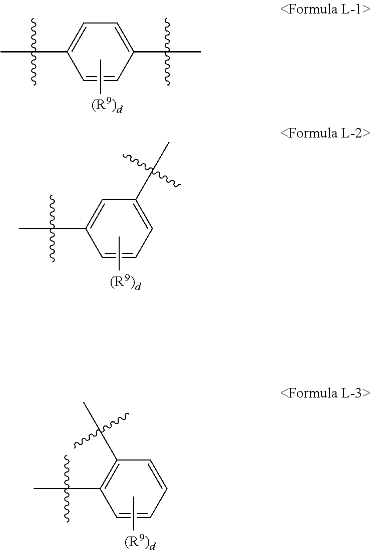
wherein,
R⁹ is selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and
d is an integer of 0 to 4, when d is an integer of 2 or more, each of R⁹s are the same as or different from each other.

5. The compound of claim 1, wherein at least one of R¹ to R⁵ is selected from the group consisting of the aryl group, the alkyl group and cycloalkyl group.

6. The compound of claim 1, wherein at least one of R¹ to R⁵, Ar¹ is selected from the group consisting of Formula C-1 to Formula C-9: 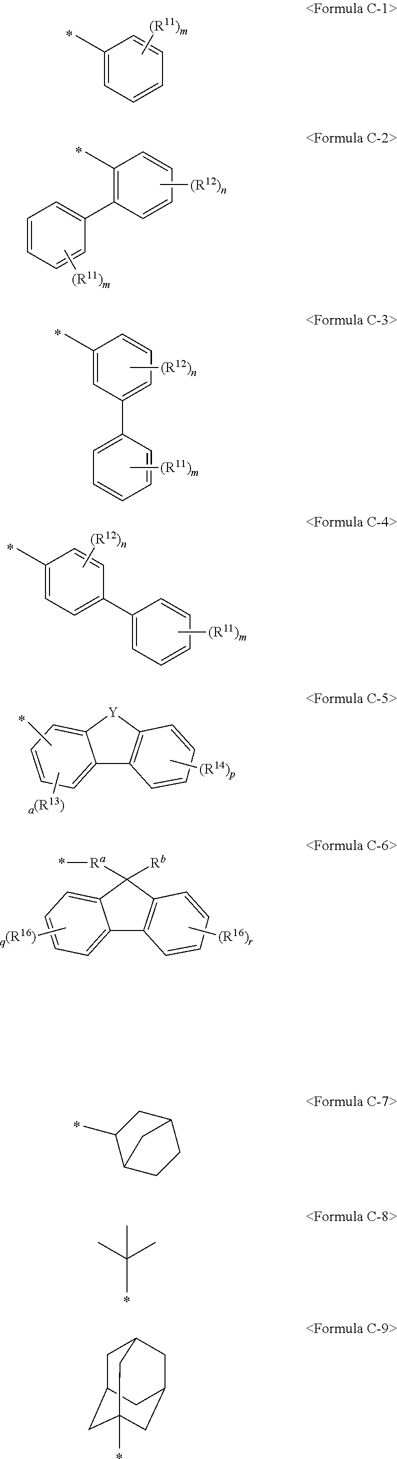
wherein,
Y is O, S, C(R²¹)(R²²) or N(R²³),
R¹¹ to R¹⁶, R²¹, R²², R^{b} are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and adjacent groups may be bonded to each other to form a ring, and R²¹ and R²² may be bonded to each other to form a ring,
R^{a} is selected from the group consisting of a single bond, a C₁-C₂₀ alkylene group, a C₂-C₂₀ alkenylene group, a C₆-C₃₀ arylene group, a fluorenylene group, a C₂-C₃₀ heterocyclic group containing at least one heteroatom of O, N, S, Si and P, and a C₃-C₃₀ aliphatic ring group, and R^{a} and R^{b} may be bonded to each other to form a ring,
m is an integer of 0 to 5, n, p, q and r are each an integer of 0 to 4, o is an integer of 0 to 3, and when each of these is an integer of 2 or more, each of R″s, each of R¹²s, each of R¹³s, each of R¹⁴s, each of R¹⁵s, each of R¹⁶s are the same as or different from each other, and
R²³ is selected from the group consisting of a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group.

7. The compound of claim 1, wherein Formula 1 is represented by one of Formula 1-9 to Formula 1-12: 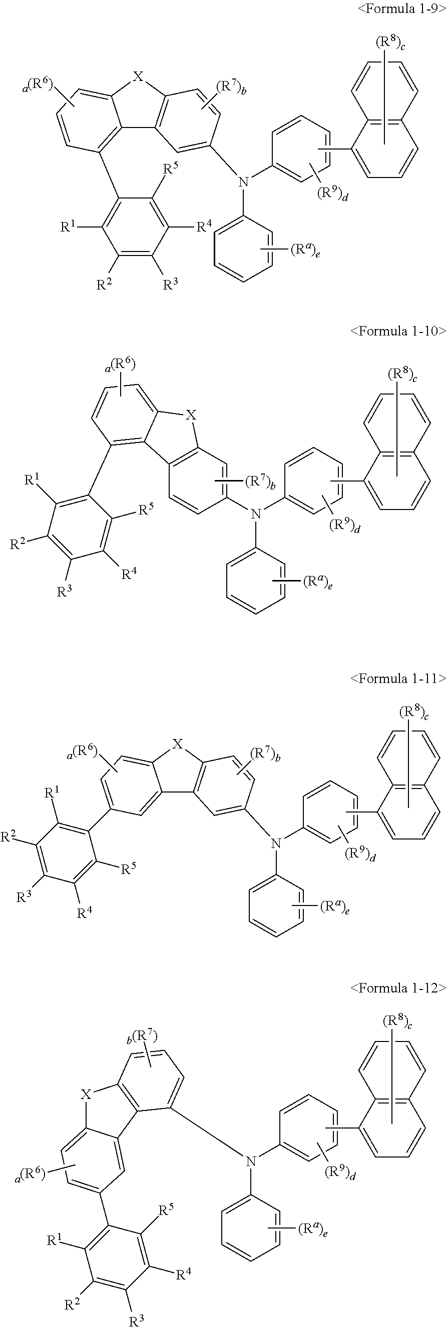
wherein, X, R¹ to R⁸, and a to c are the same as defined in claim 1,
R⁹ and R are independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and adjacent R^{a}s may be bonded to each other to form a ring, and
d is an integer of 0 to 4, e is an integer of 0 to 5, when each of these is an integer of 2 or more, each of R⁹s, each of R^{a}s are the same as or different from each other.

8. The compound of claim 1, wherein the compound represented by Formula 1 is one of the following compounds:

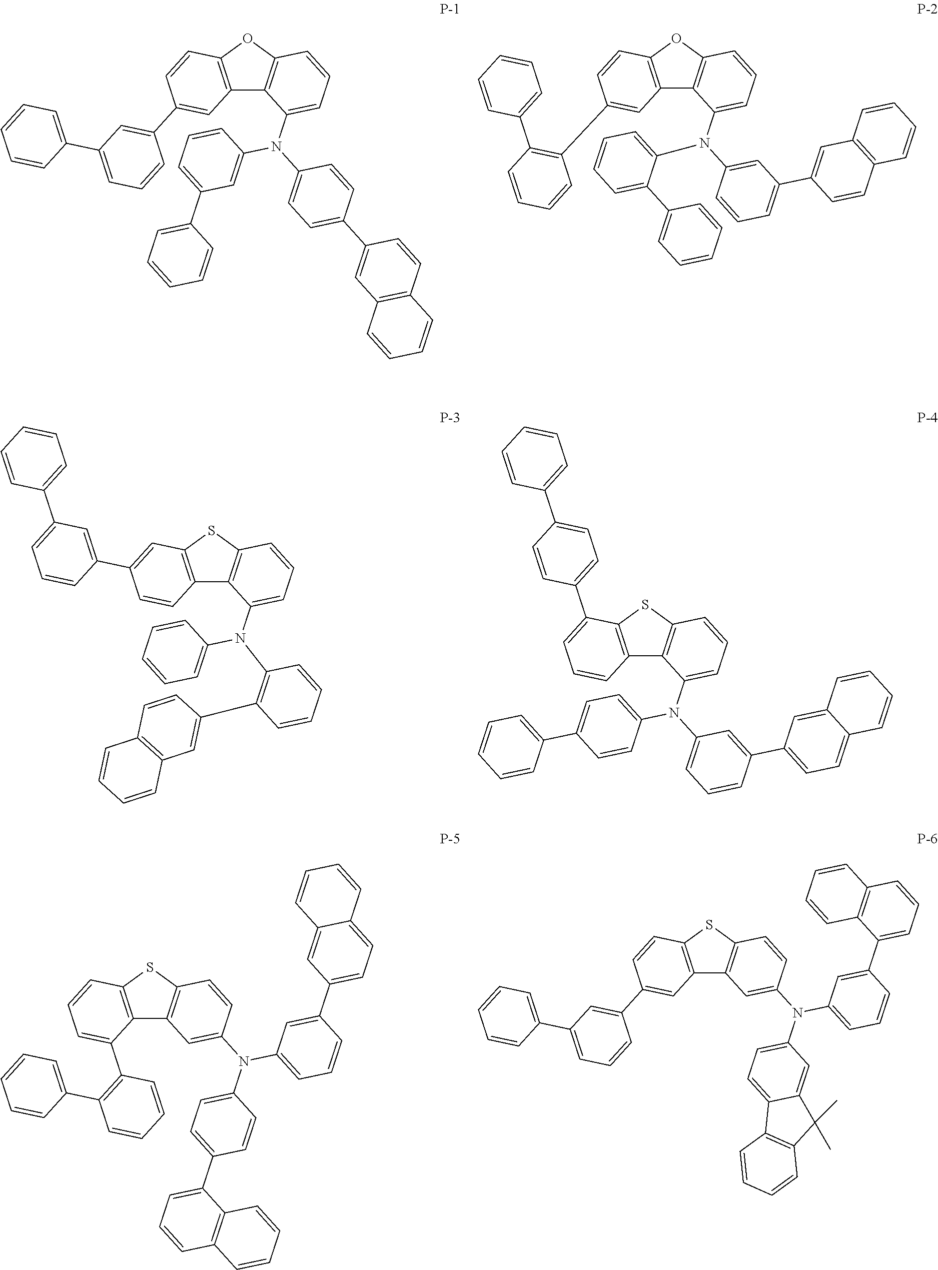

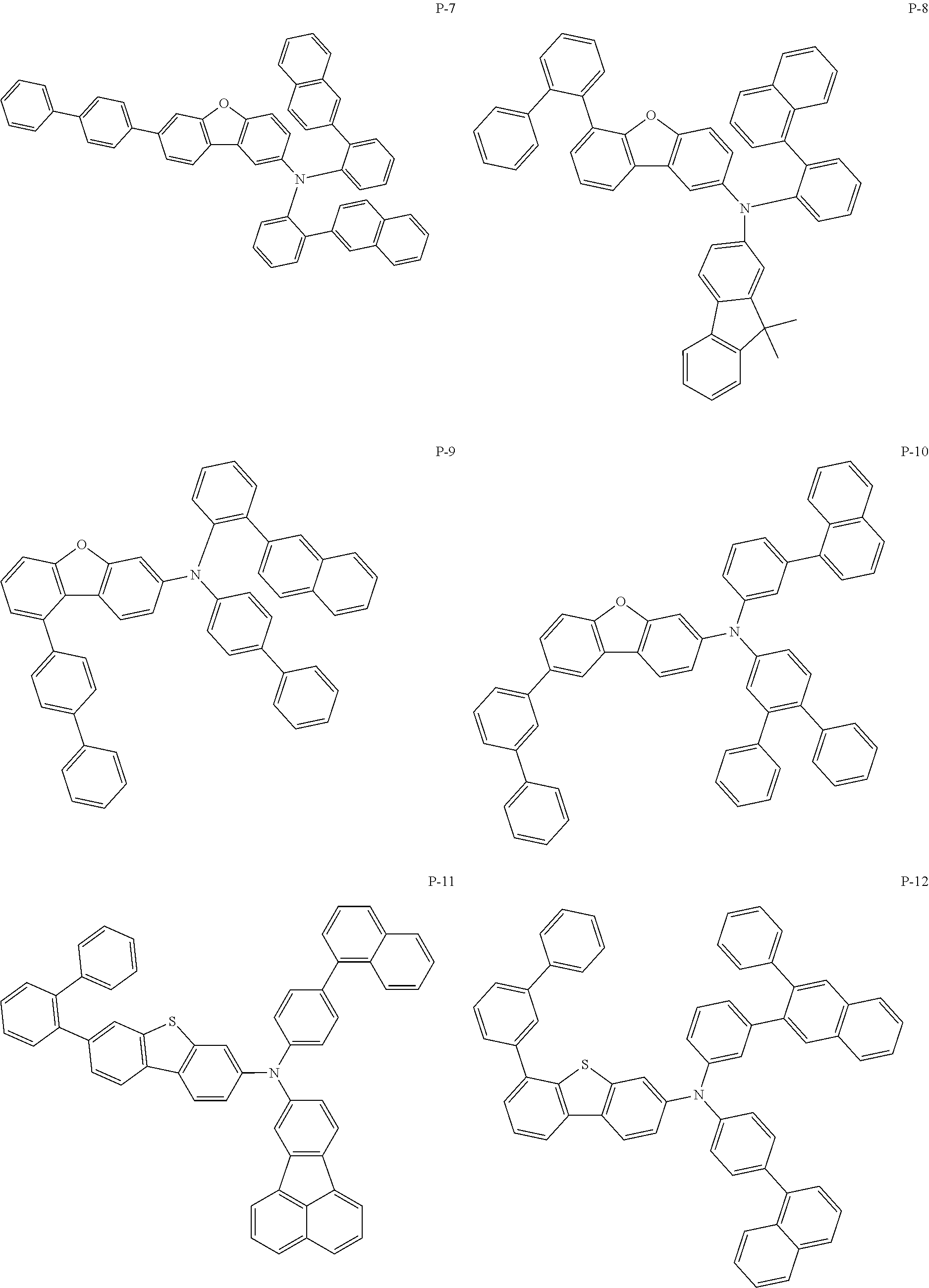

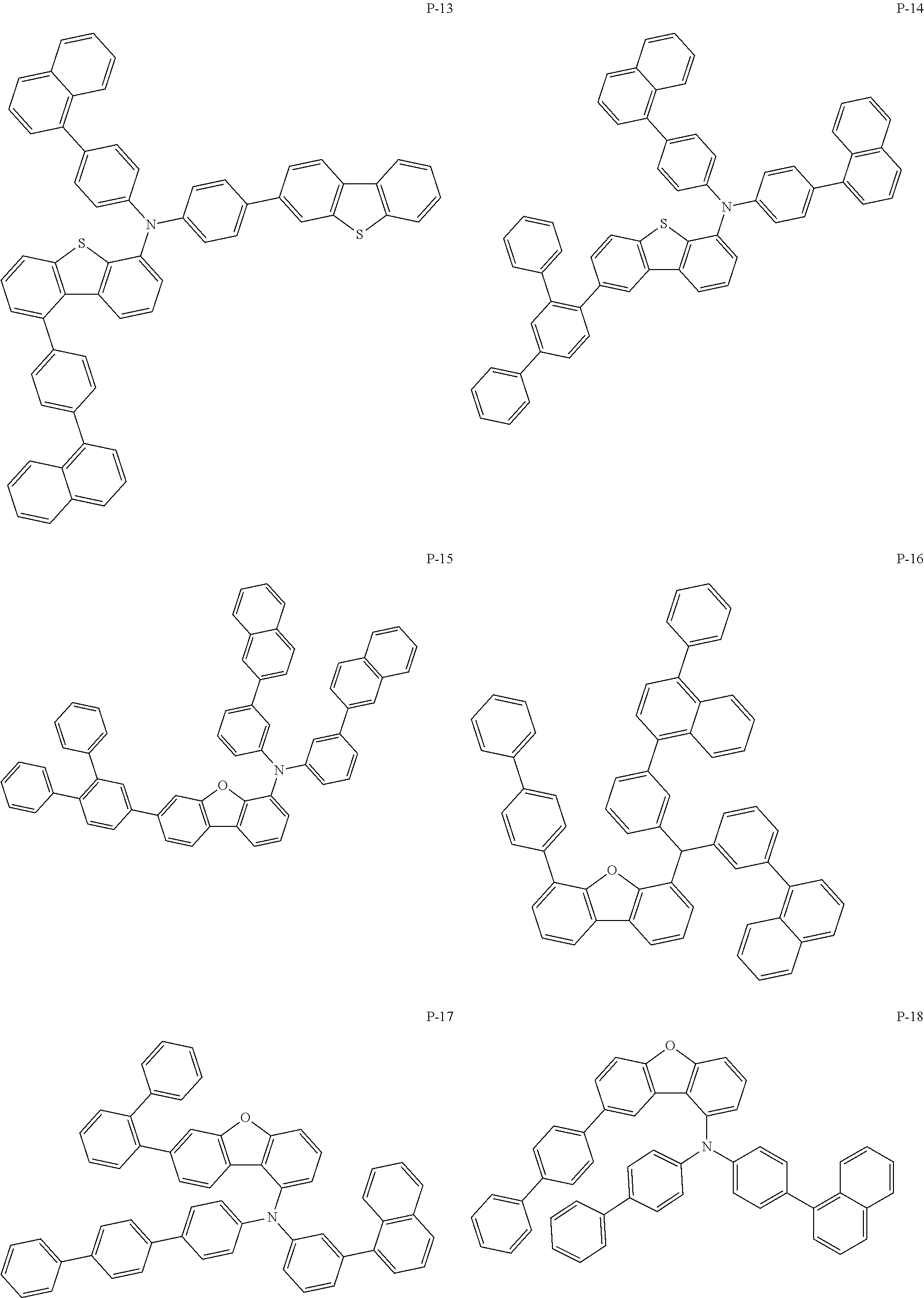

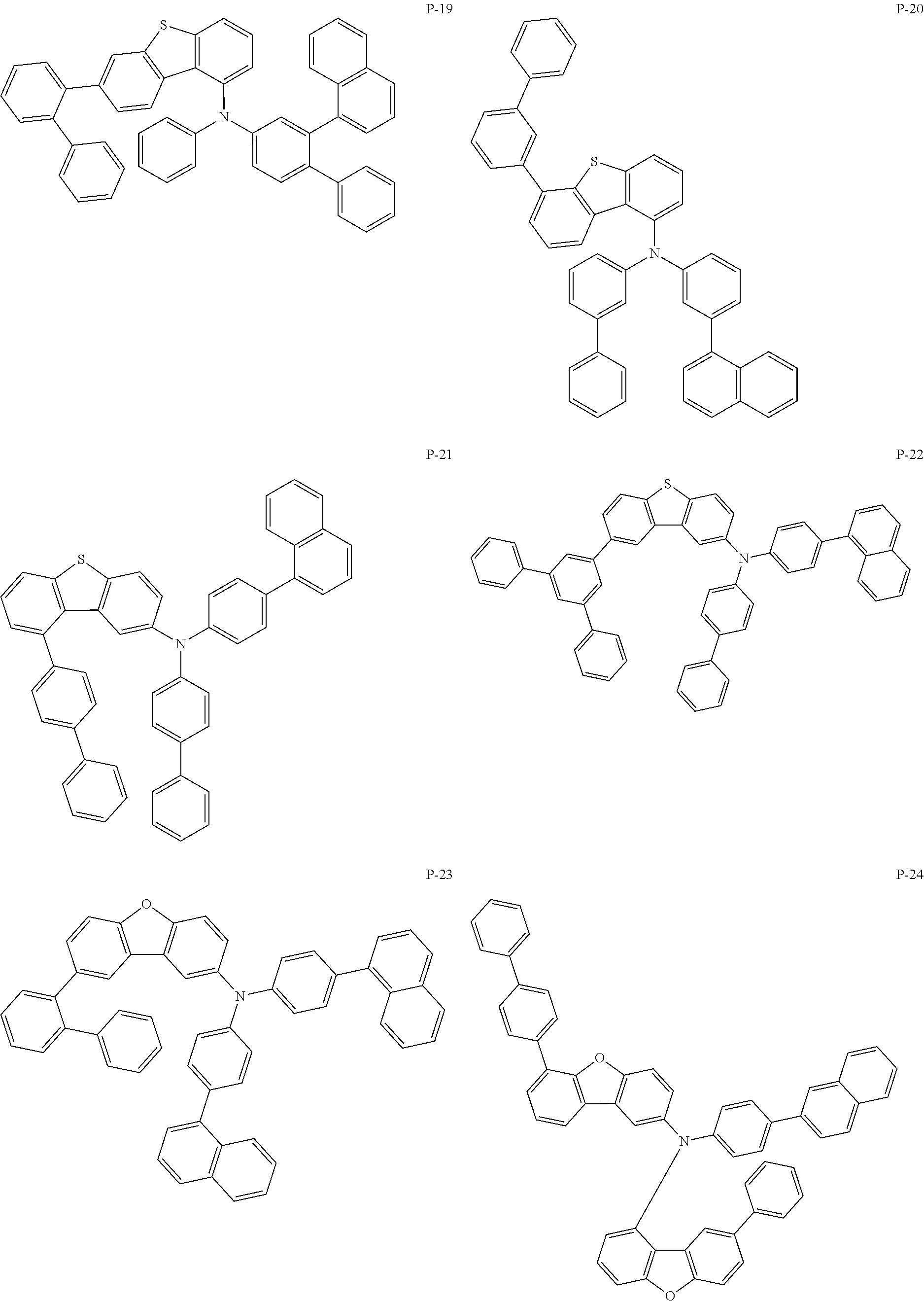

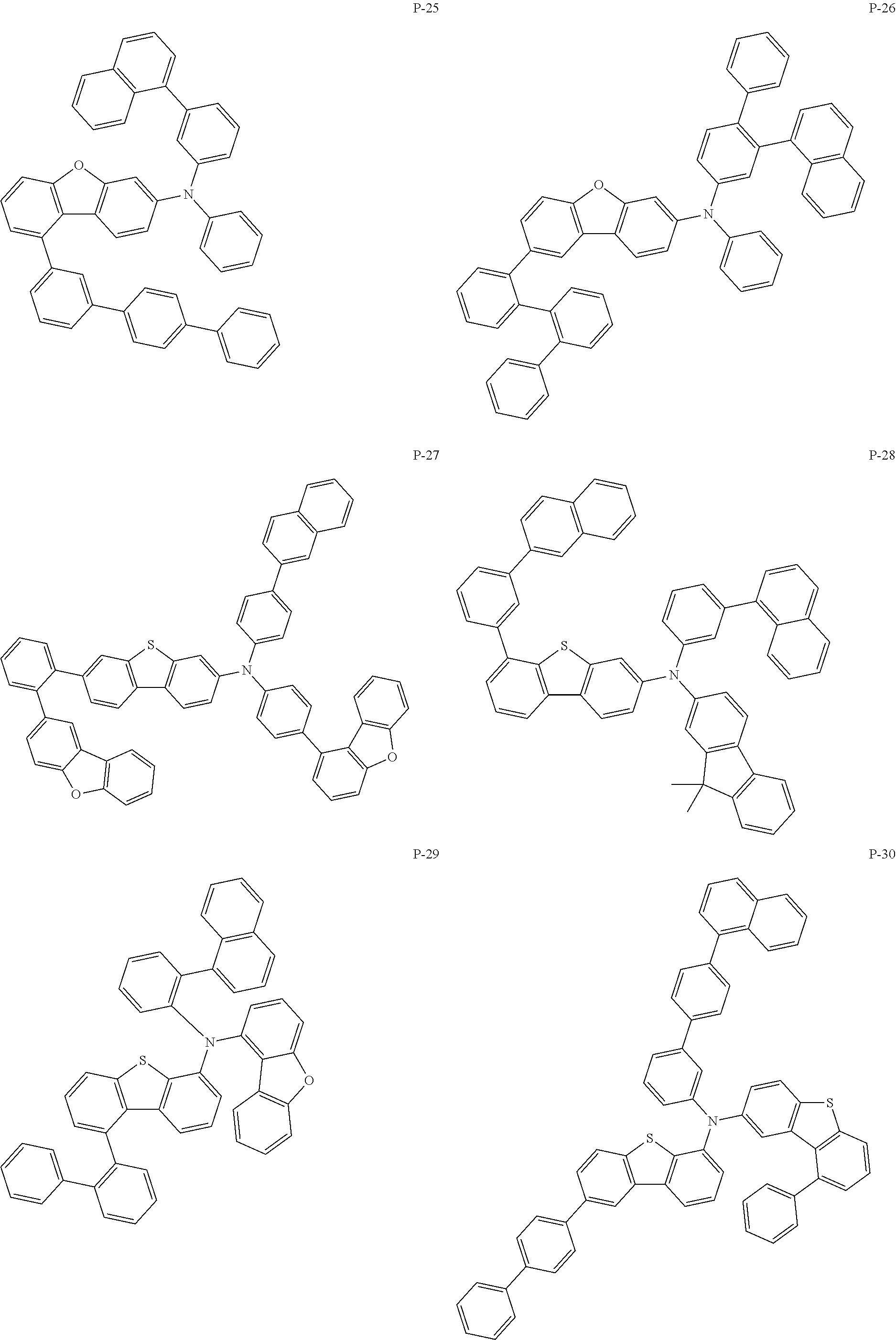

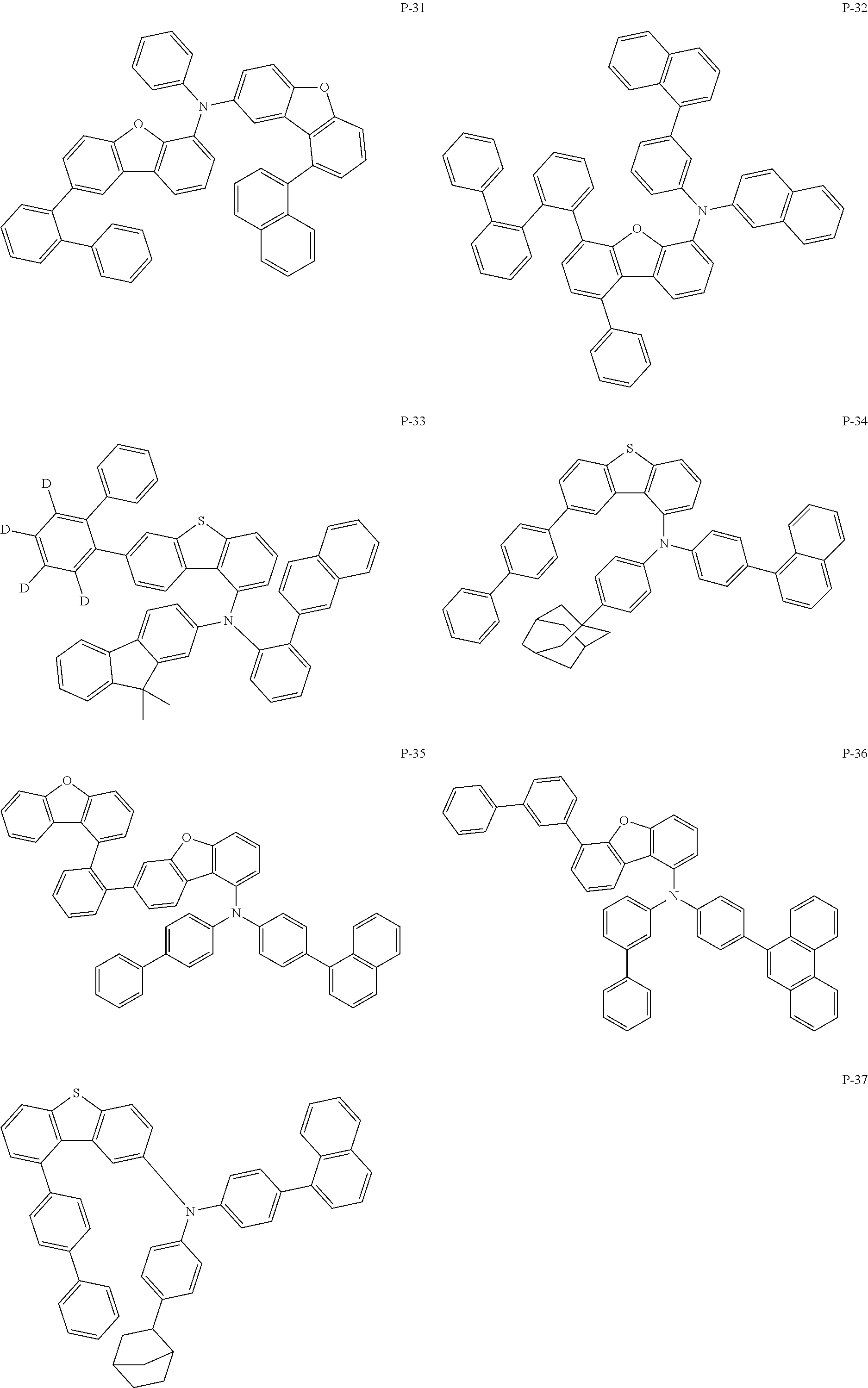

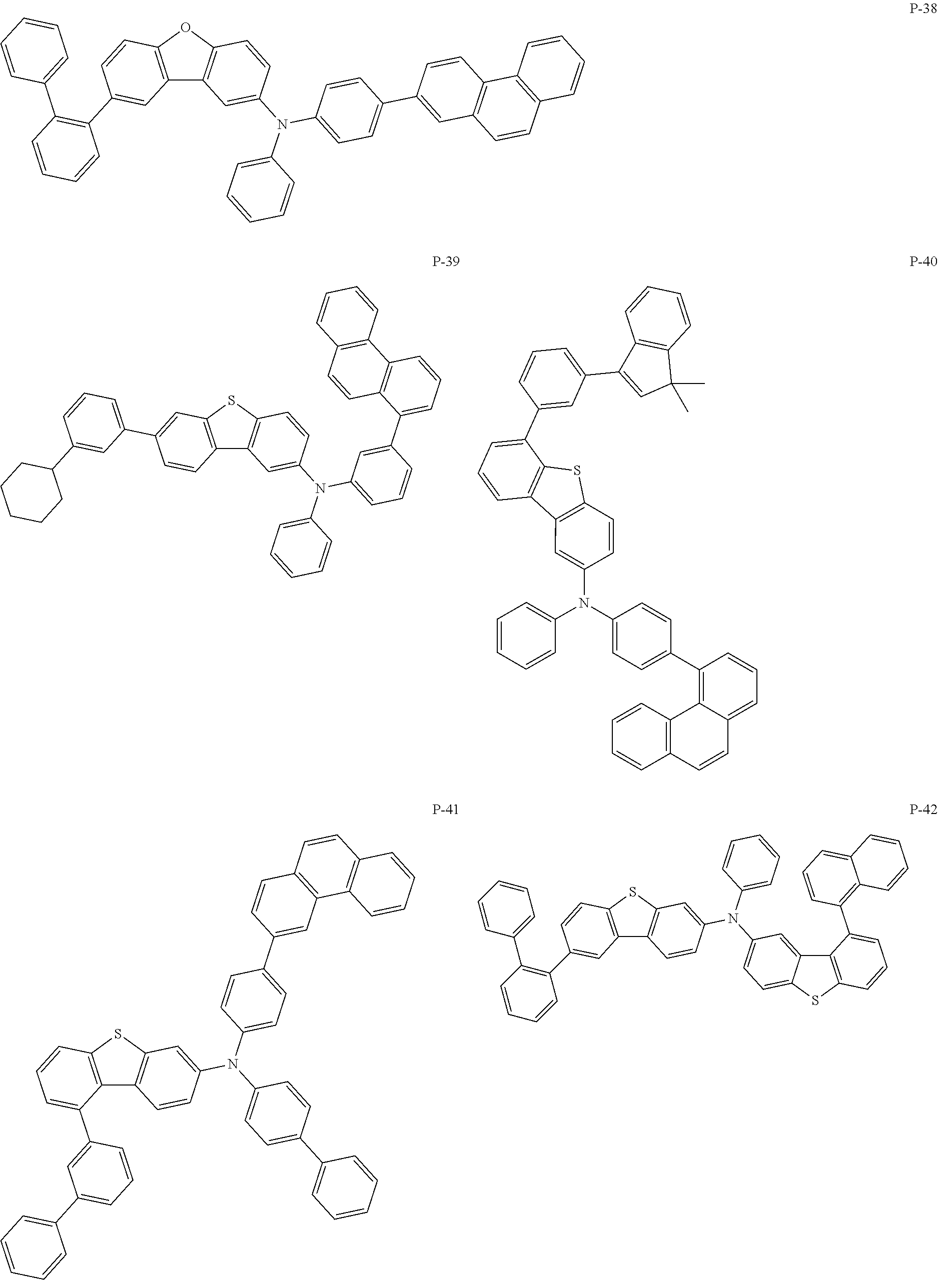 [SMILES: c1ccc(-c2ccccc2-c2ccc3oc4ccc(N(c5ccccc5)c5ccc(-c6ccc7c(ccc8ccccc87)c6)cc5)cc4c3c2)cc1 ; c1ccc(N(c2cccc(-c3cccc4c3ccc3ccccc34)c2)c2ccc3sc4cc(-c5cccc(C6CCCCC6)c5)ccc4c3c2)cc1 ; CC1(C)C=C(c2cccc(-c3cccc4c3sc3ccc(N(c5ccccc5)c5ccc(-c6cccc7ccc8ccccc8c67)cc5)cc34)c2)c2ccccc21 ; c1ccc(-c2ccc(N(c3ccc(-c4ccc5ccc6ccccc6c5c4)cc3)c3ccc4c(c3)sc3cccc(-c5cccc(-c6ccccc6)c5)c34)cc2)cc1 ; c1ccc(-c2ccccc2-c2ccc3sc4cc(N(c5ccccc5)c5ccc6sc7cccc(-c8cccc9ccccc89)c7c6c5)ccc4c3c2)cc1]

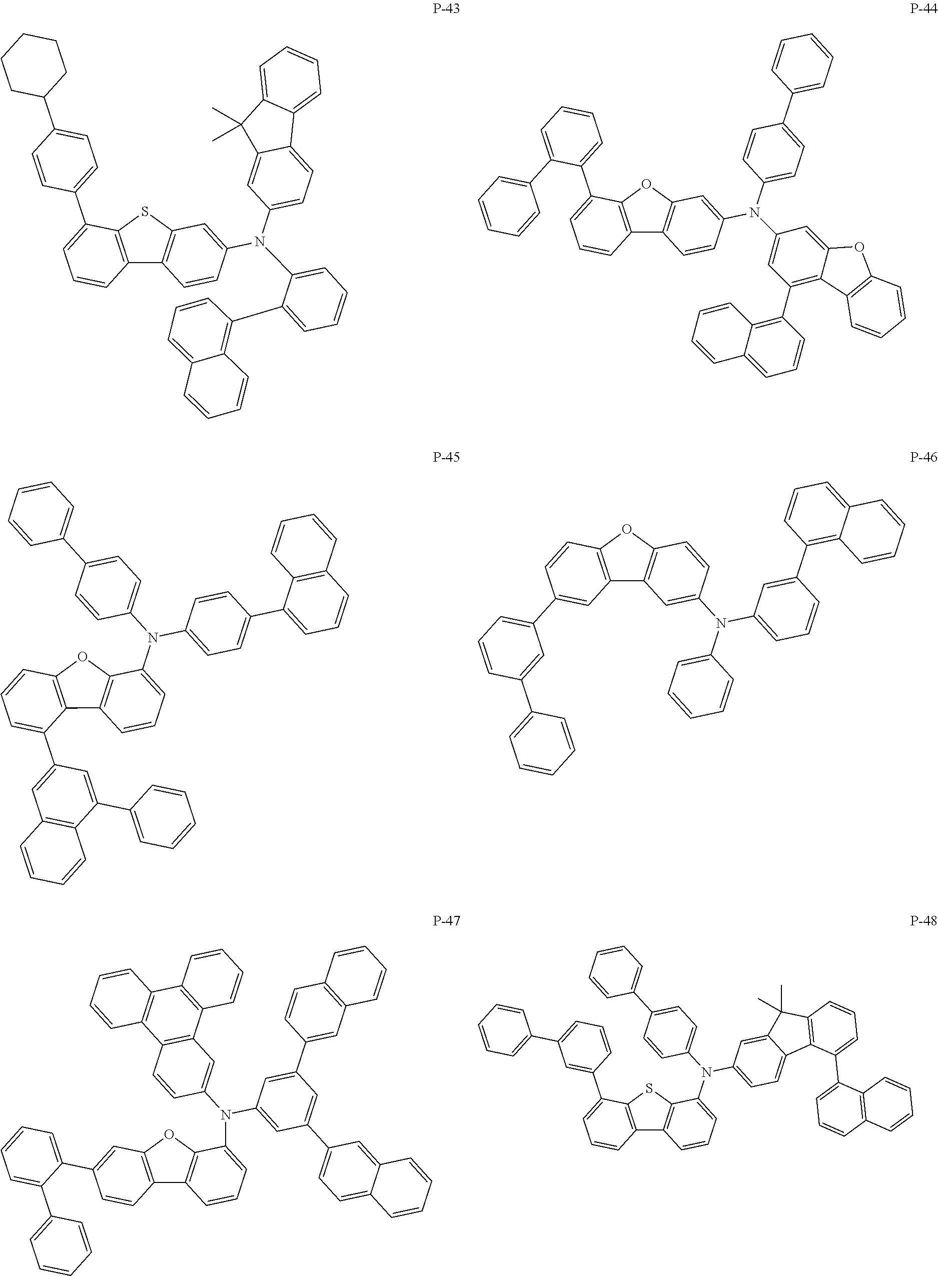

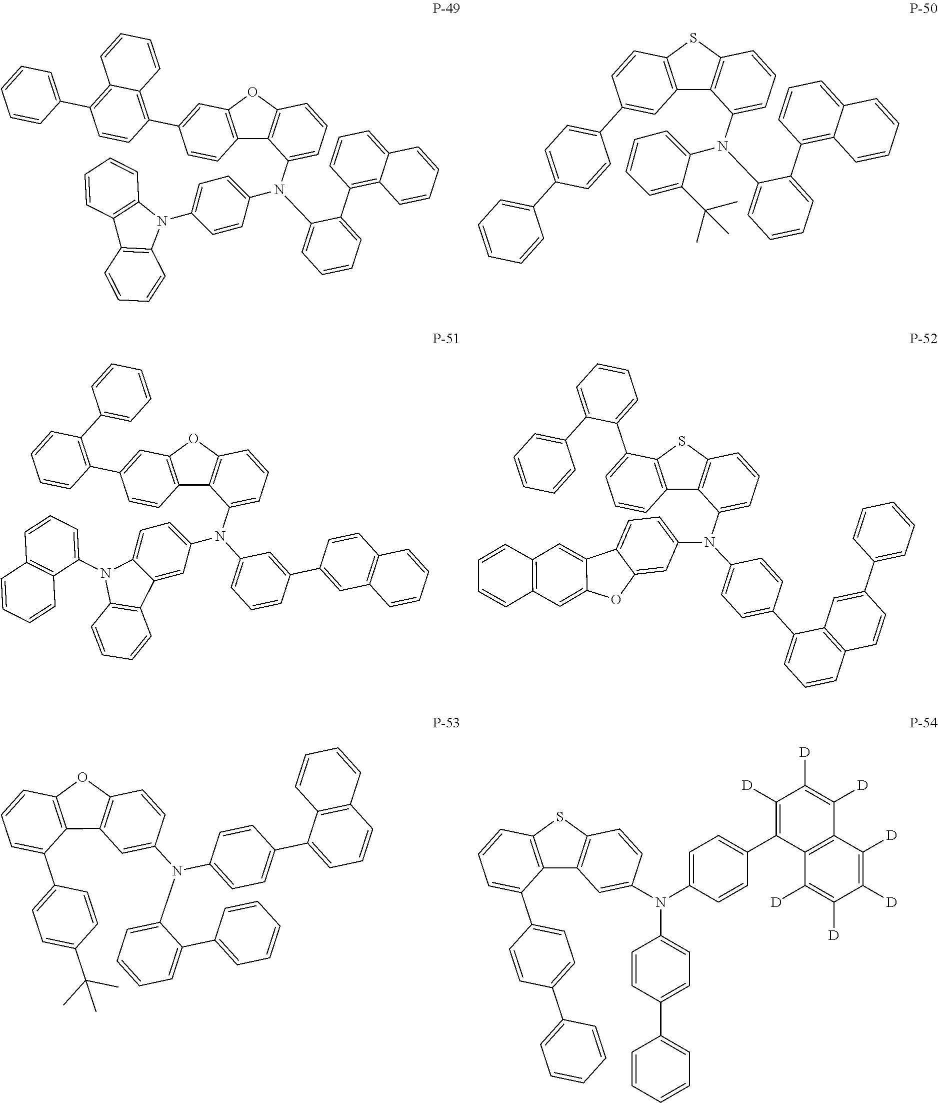

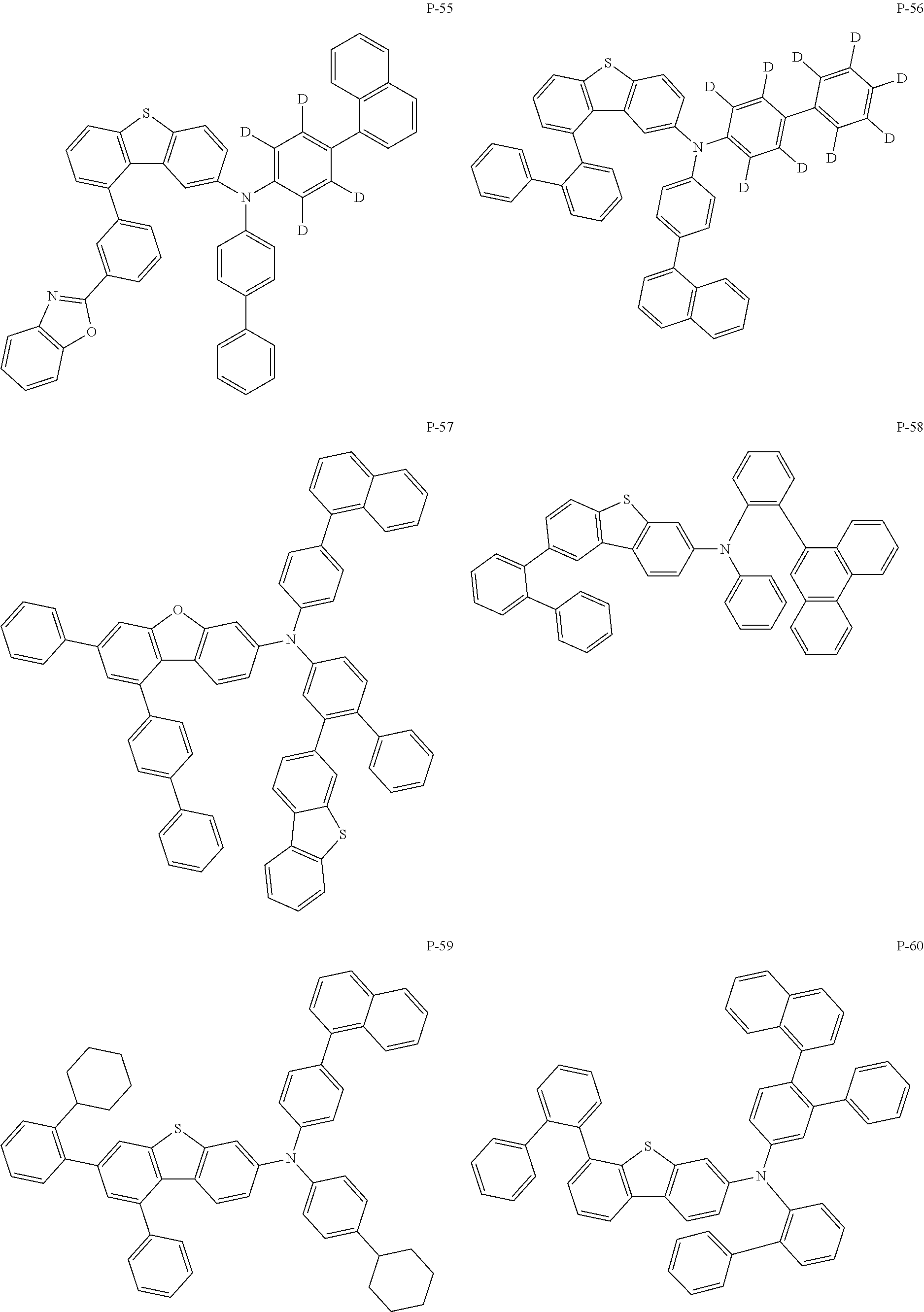

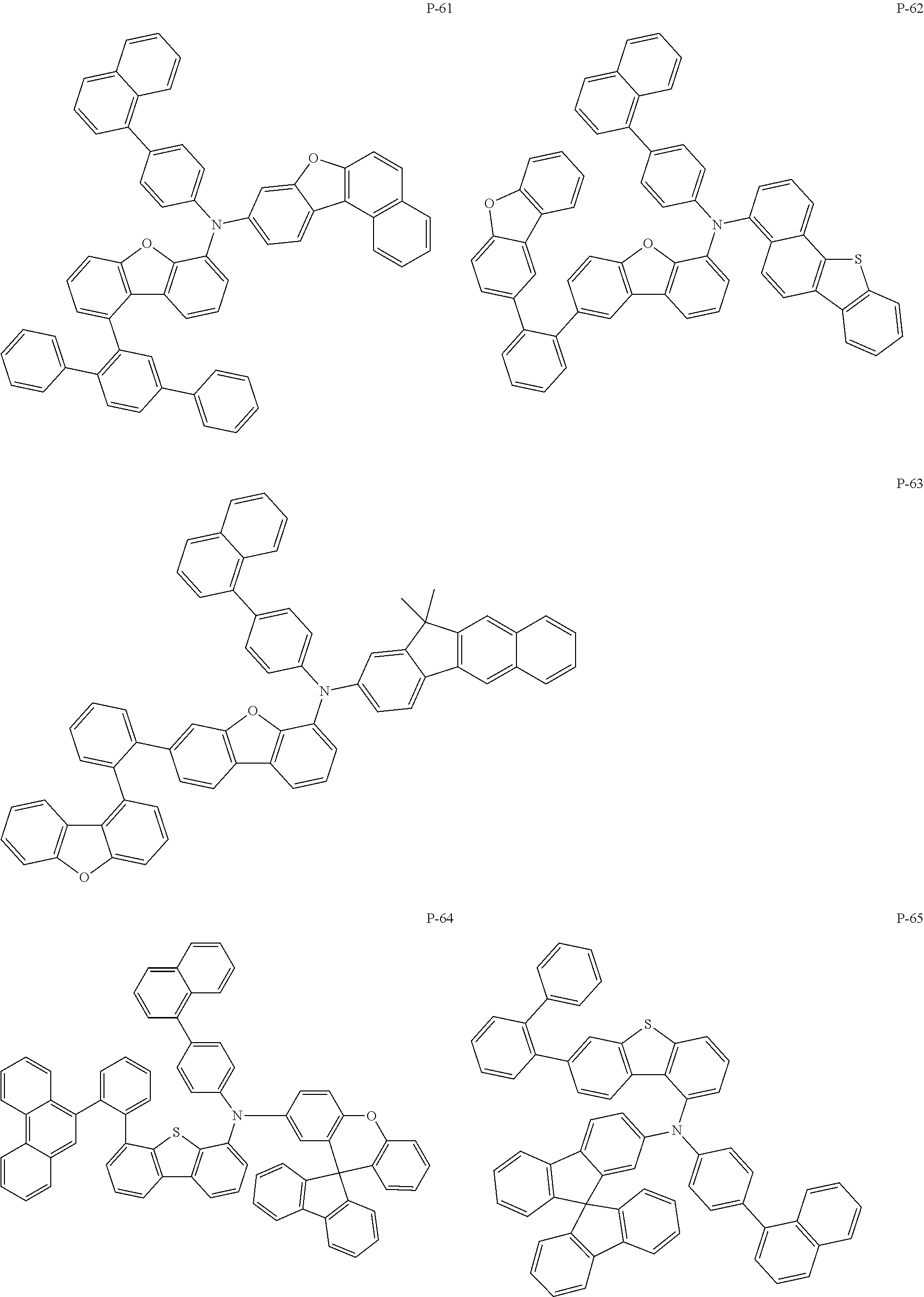

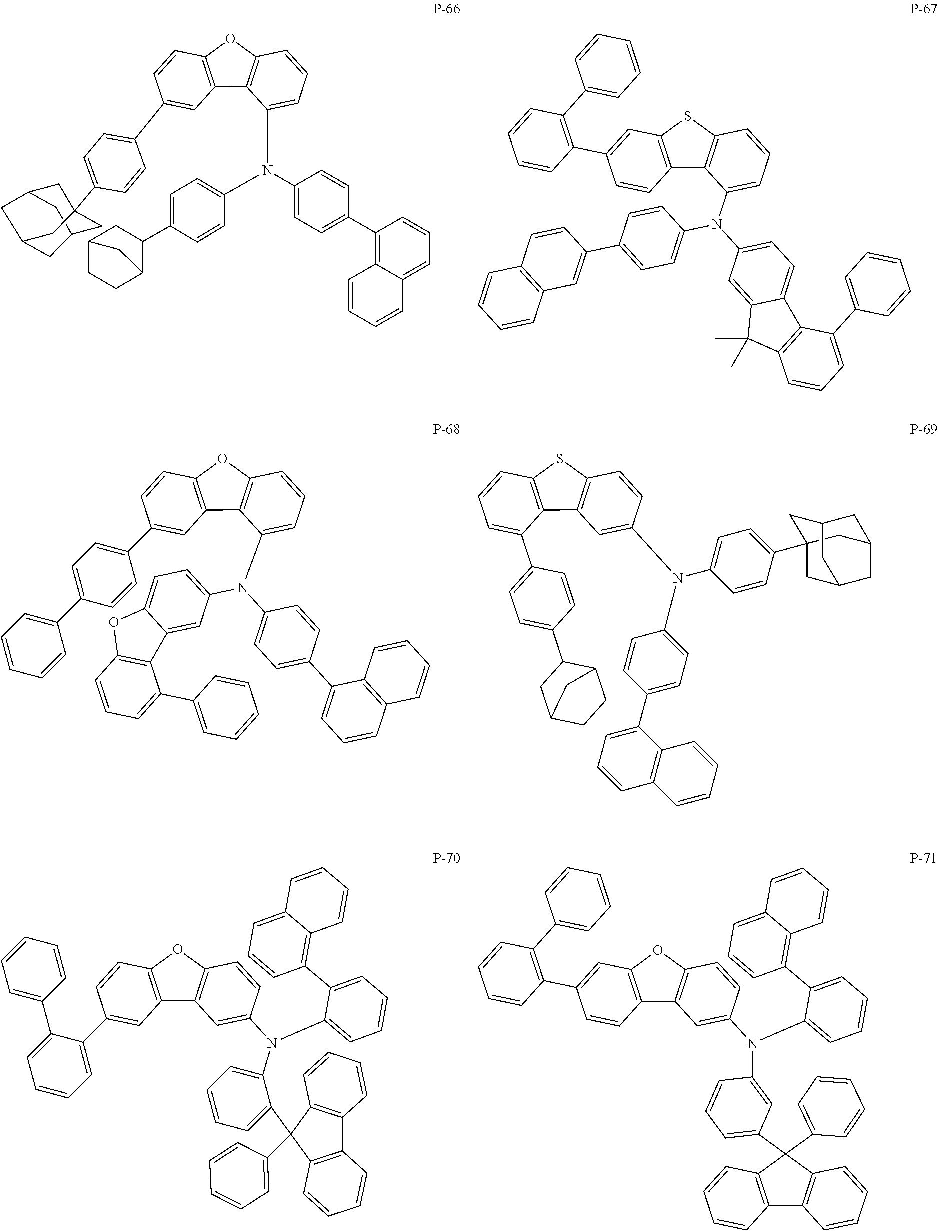

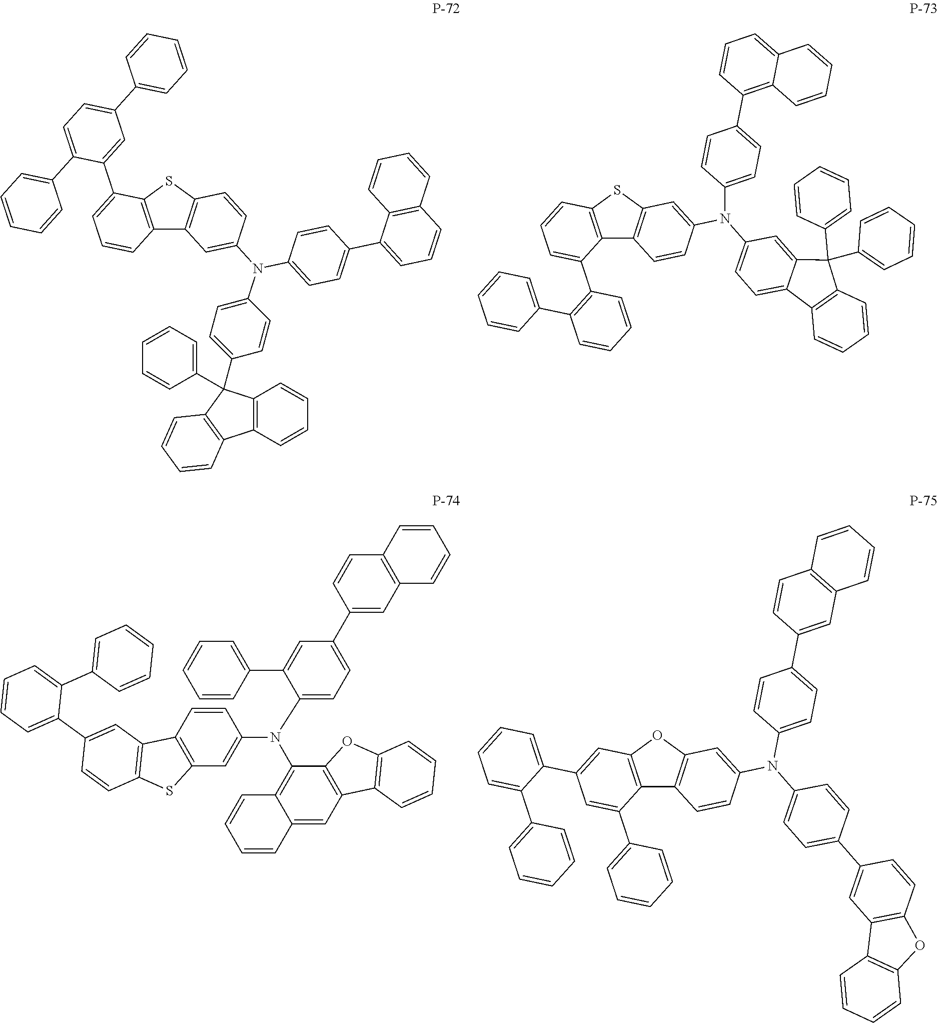

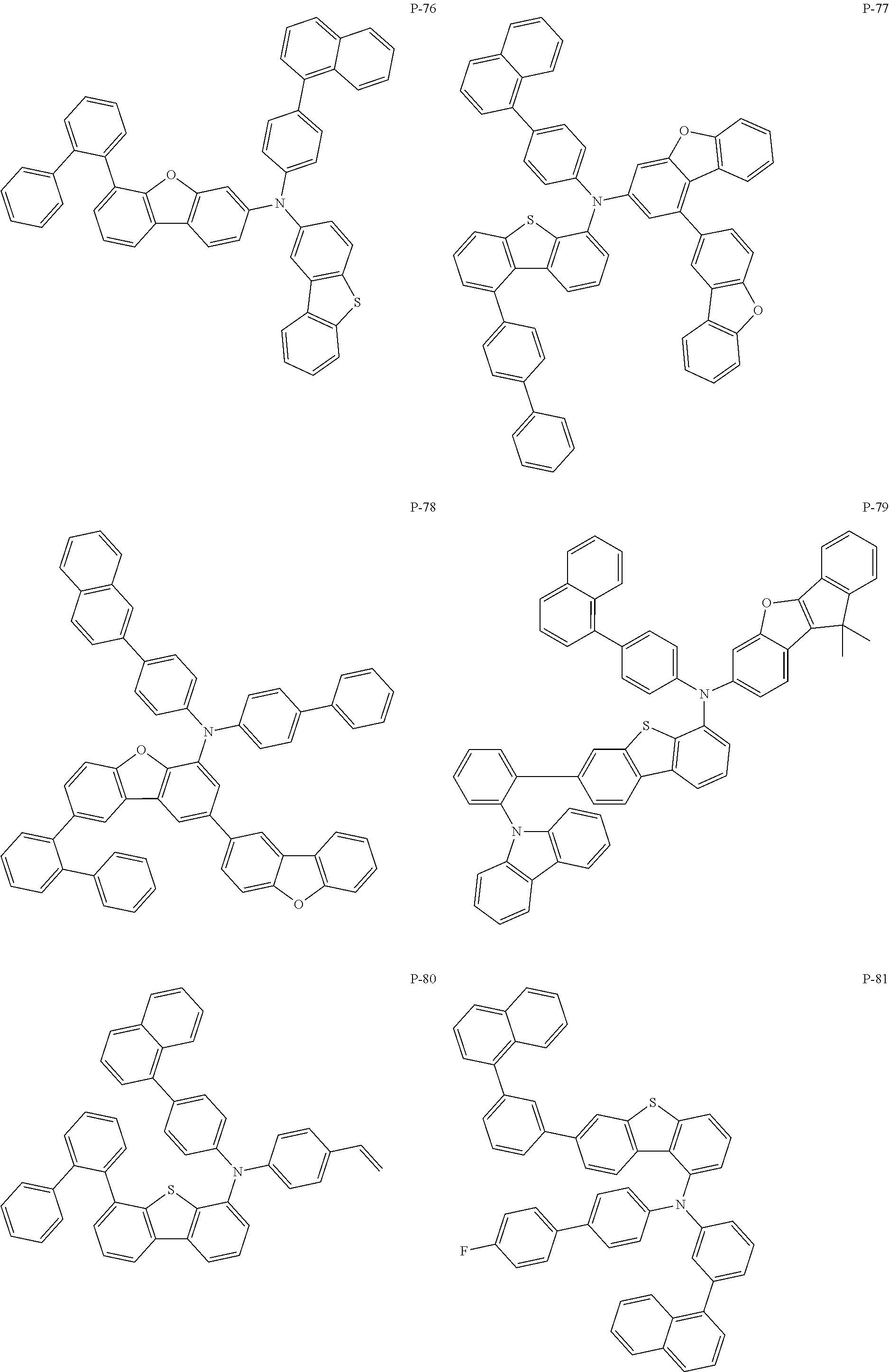

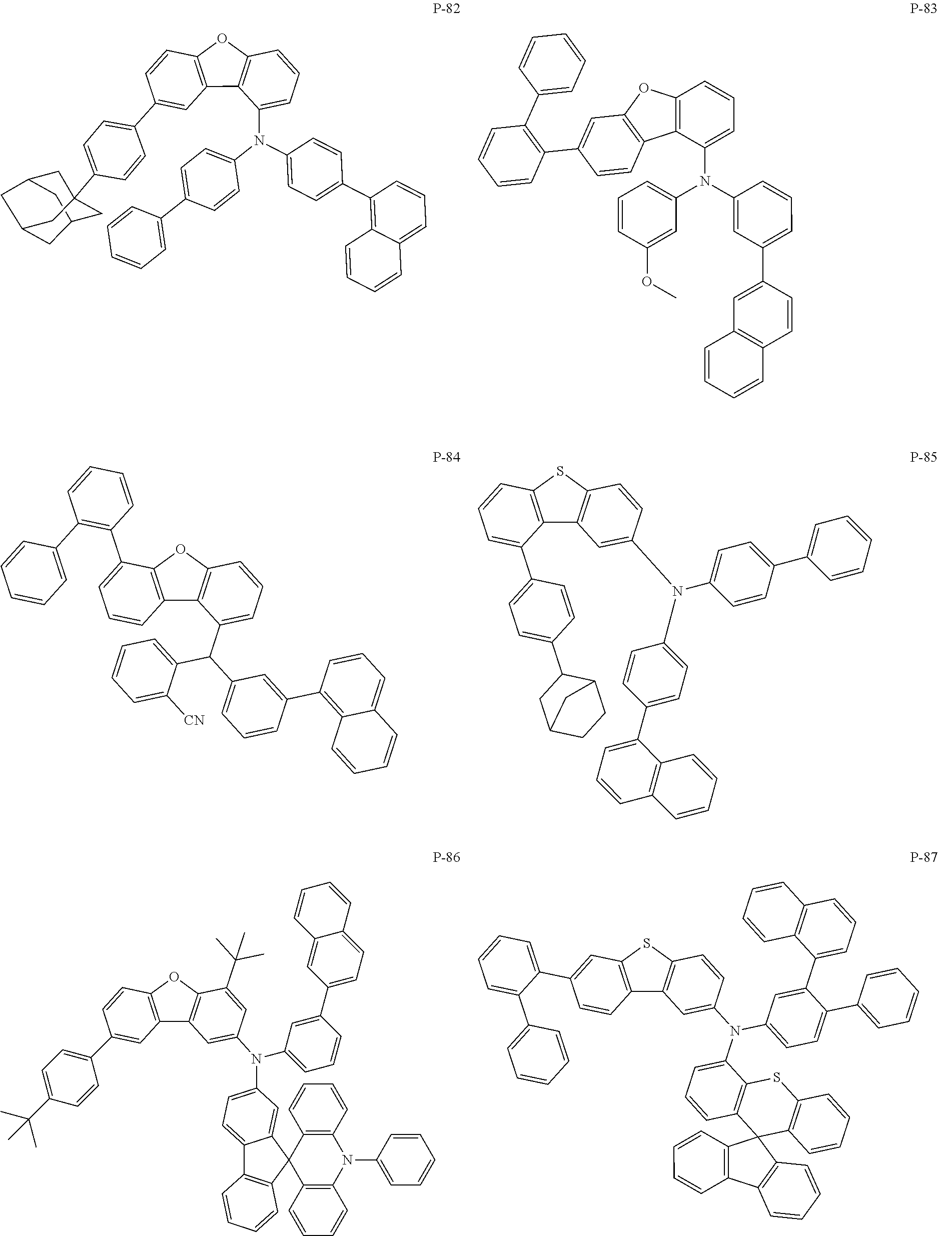

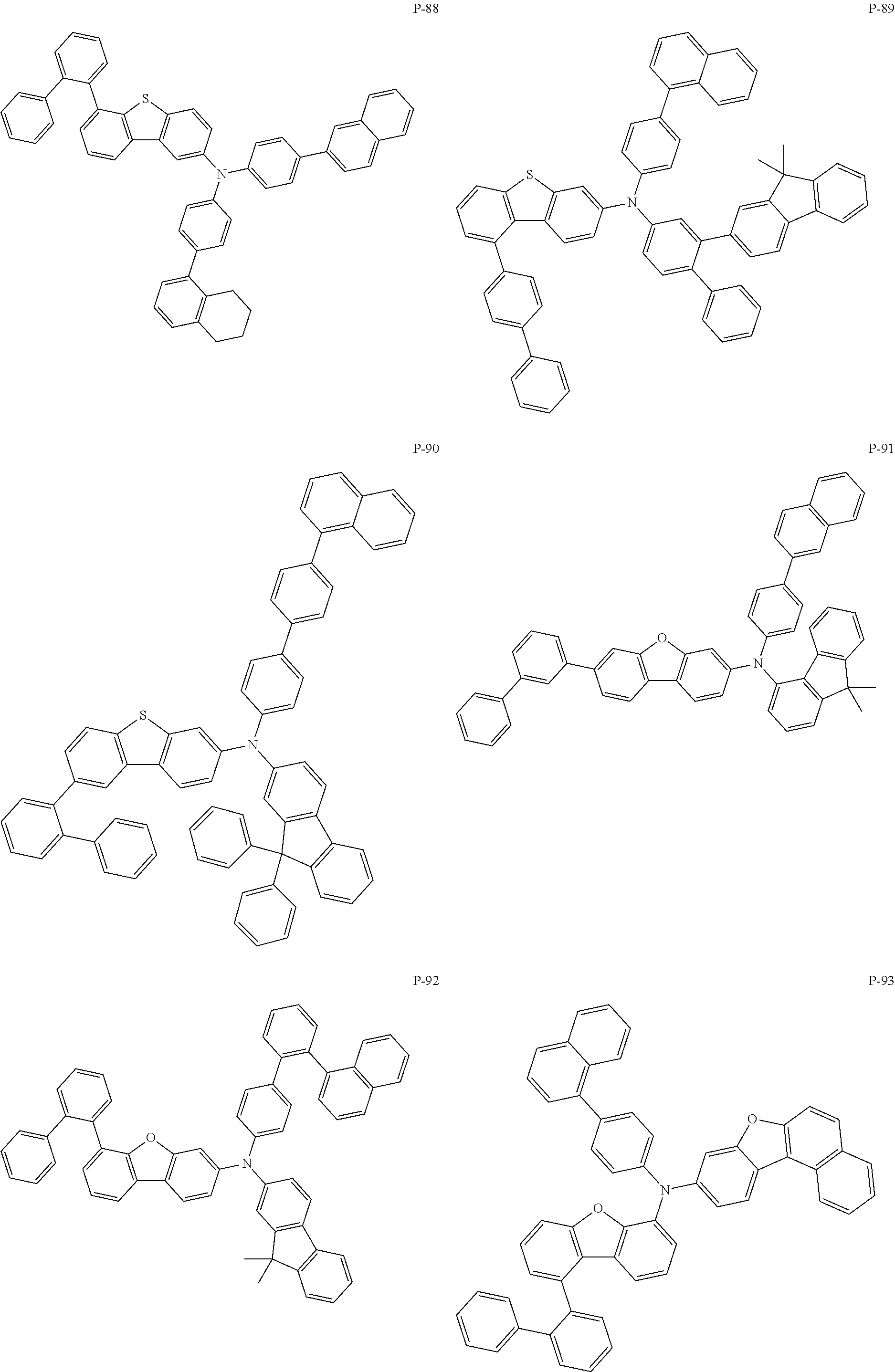

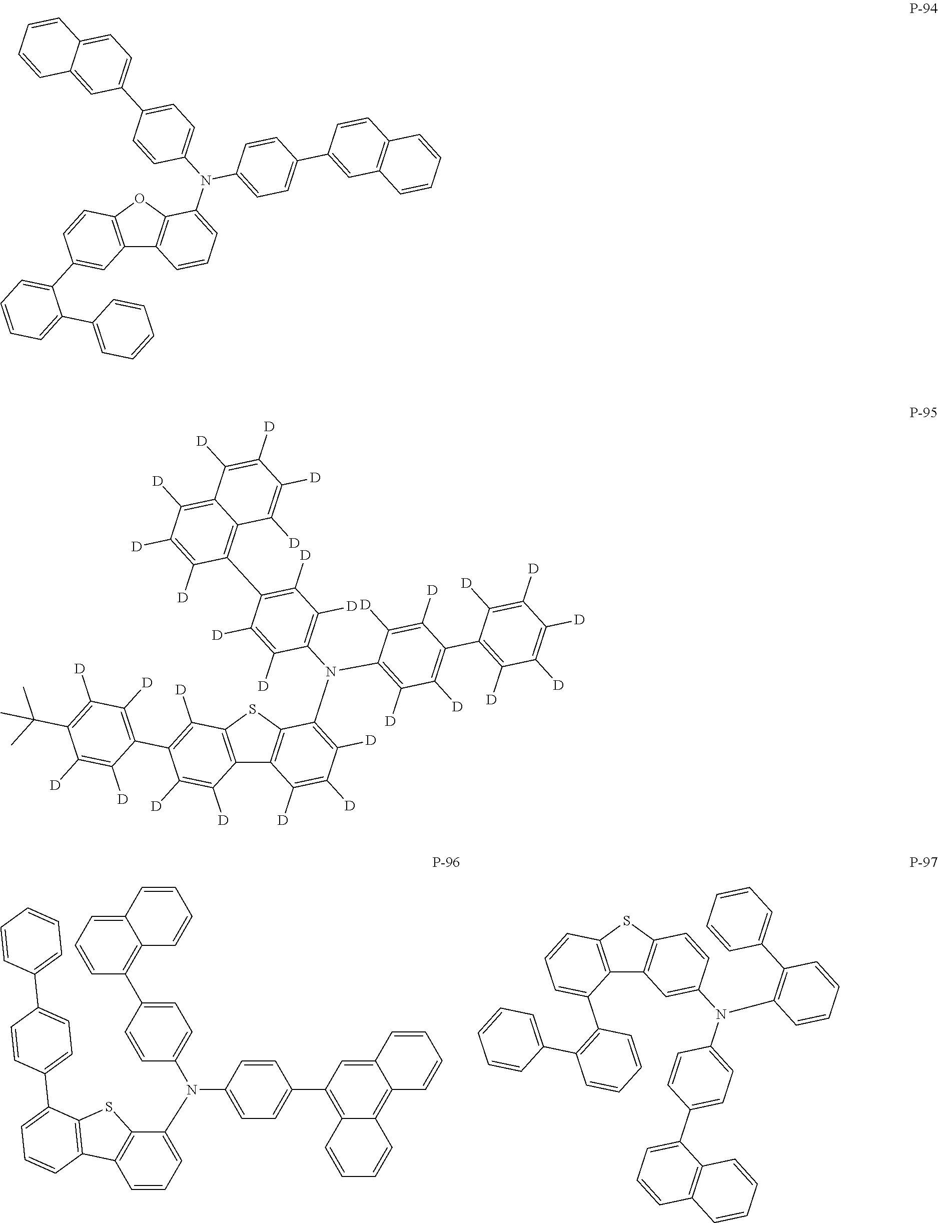

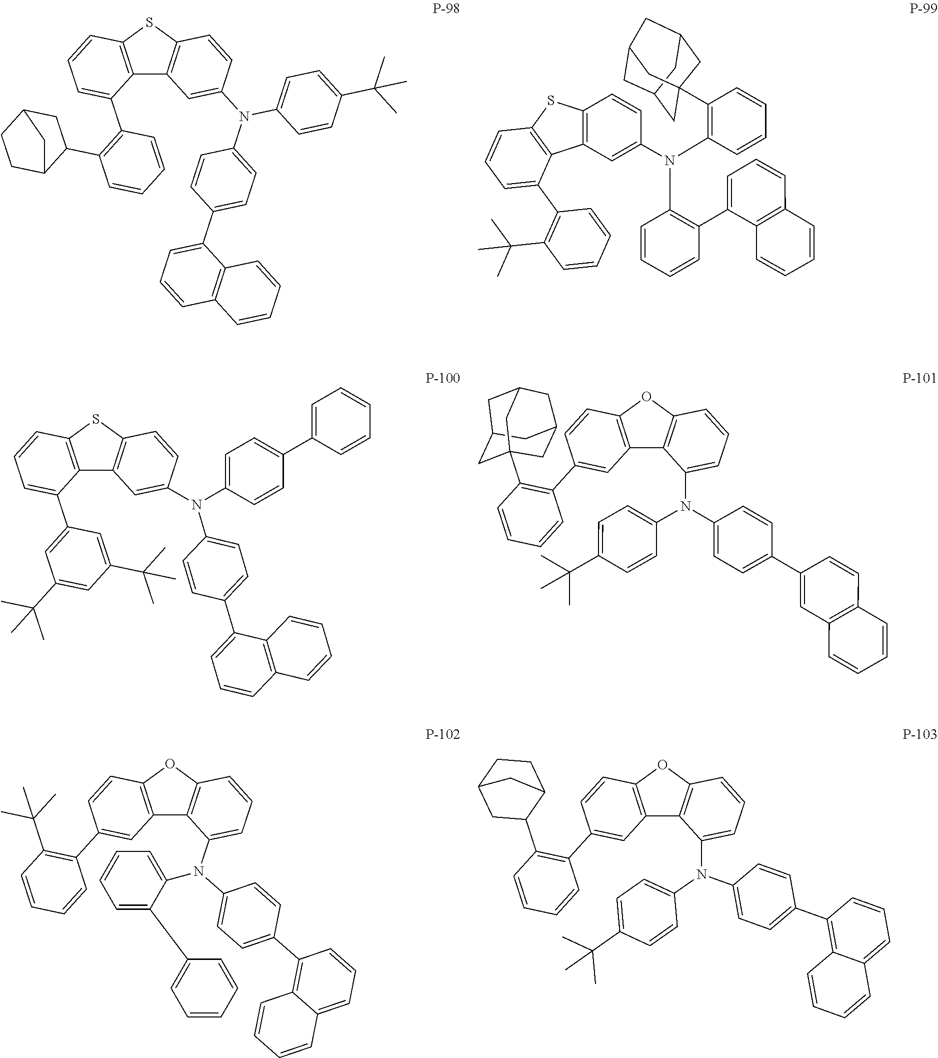

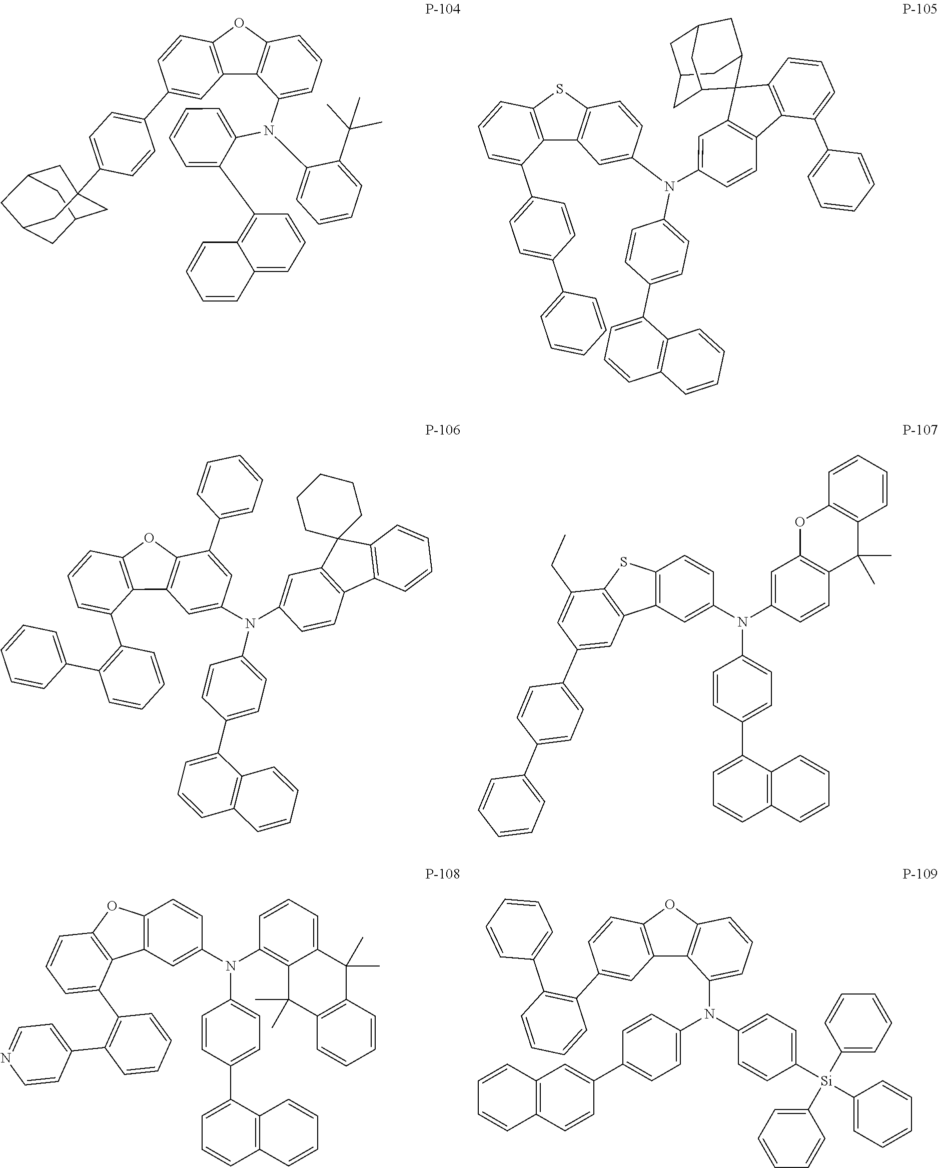

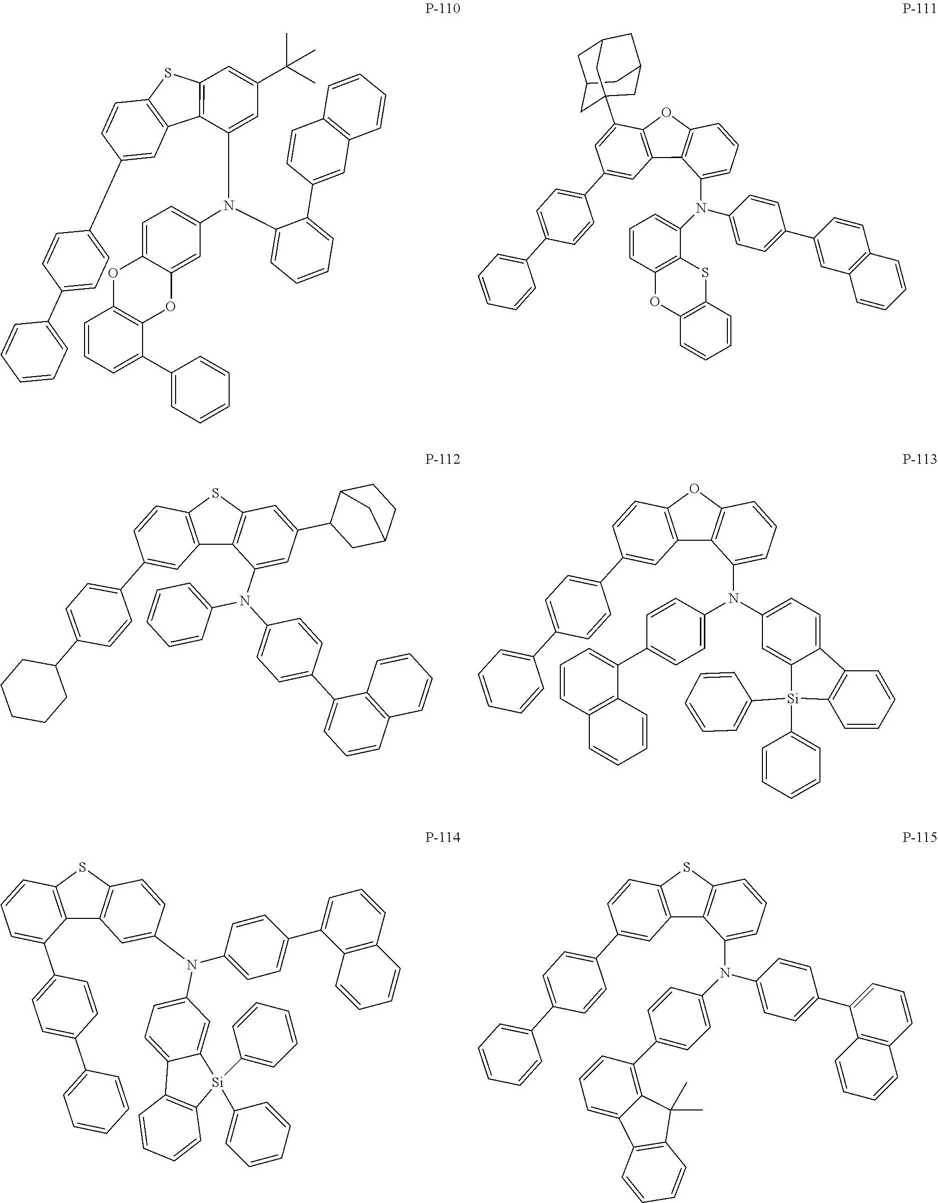

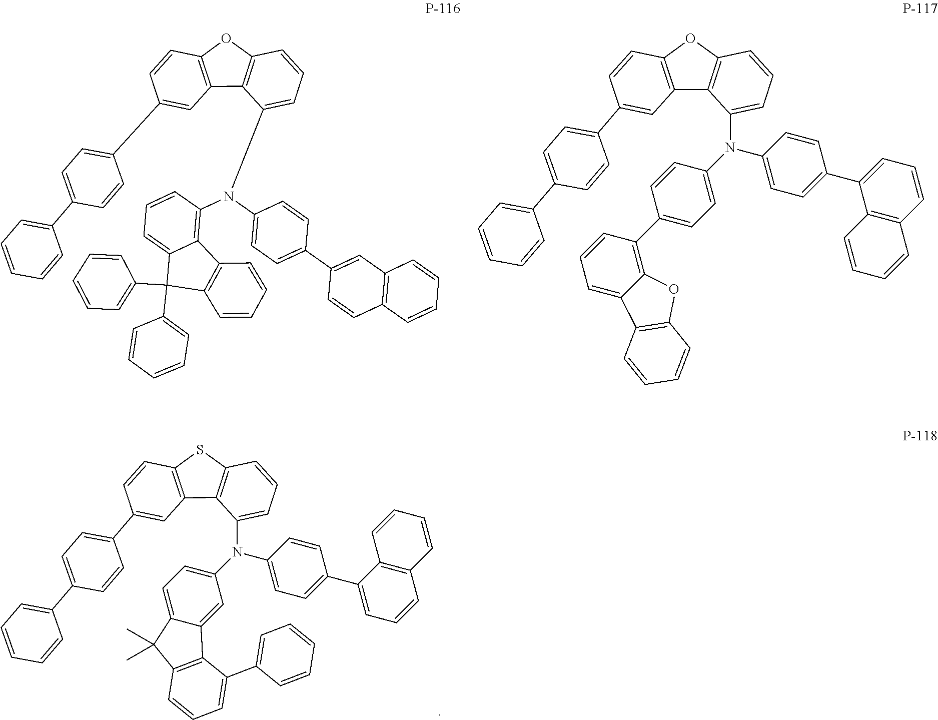 [SMILES: c1ccc(-c2ccc(-c3ccc4oc5cccc(N(c6ccc(-c7ccc8ccccc8c7)cc6)c6cccc7c6-c6ccccc6C7(c6ccccc6)c6ccccc6)c5c4c3)cc2)cc1 ; c1ccc(-c2ccc(-c3ccc4oc5cccc(N(c6ccc(-c7cccc8ccccc78)cc6)c6ccc(-c7cccc8c7oc7ccccc78)cc6)c5c4c3)cc2)cc1 ; CC1(C)c2ccc(N(c3ccc(-c4cccc5ccccc45)cc3)c3cccc4sc5ccc(-c6ccc(-c7ccccc7)cc6)cc5c34)cc2-c2c(-c3ccccc3)cccc21]

9. An organic electric element comprising a first electrode, a second electrode, and an organic material layer between the first electrode and the second electrode, wherein the organic material layer comprises the compound of Formula 1 of claim 1.

10. The organic electric element of claim 9, wherein the organic material layer comprises a light emitting layer, and a hole transport zone between the first electrode and the light emitting layer, and the compound of Formula 1 is comprised in the hole transport zone.

11. The organic electric element of claim 9, wherein the hole transport zone comprises an emission-auxiliary layer and the emission-auxiliary layer comprises the compound of Formula 1.

12. The organic electric element of claim 9, wherein the organic material layer comprises two or more stacks, and the stacks each comprise a hole transport layer, a light-emitting layer and an electron transport layer formed sequentially on the first electrode.

13. The organic electric element of claim 12, wherein the stack further comprises an emission-auxiliary layer between the hole transport layer and the light-emitting layer.

14. The organic electric element of claim 12, wherein the organic material layer further comprises a charge generation layer between the two or more stacks.

15. The organic electric element of claim 9, wherein the organic electric element further comprises a layer for improving luminous efficiency, and the layer for improving luminous efficiency is formed on one side of both sides of the first electrode or the second, and the one side not facing the organic material layer.

16. An electronic device comprising a display device and a control unit for driving the display device, wherein the display device comprises the organic electric element of claim 9.

17. The electronic device of claim 16, wherein the organic electric element is selected from the group consisting of an organic electroluminescent element, an organic solar cell, an organic photo conductor, an organic transistor, an element for monochromatic illumination and a quantum dot display.
